(19) United States
(12) Patent Application Publication
(10) Pub. No.: US 2003/0059794 A1
(43) Pub. Date: Mar. 27, 2003

(54) Adenoviral library assay for E2F regulatory genes and methods and compositions for screening compounds

(76) Inventors: Ronald Vogels, Linschoten (NL); Abraham Bout, Moerkapelle (NL); Helmuth van Es, Hoofddorp (NL); Govert Schouten, Leiden (NL); Rene Bernards, Abcoude (NL); Godefridus A.M. Michiels, Leiderdorp (NL); Reginald C.X. Brys, Kessel-Lo (BE); Peter Herwig Maria Tomme, Gent (BE)

Correspondence Address: SYNNESTVEDT & LECHNER, LLP, 2600 ARAMARK TOWER, 1101 MARKET STREET, PHILADELPHIA, PA, 191072950

(21) Appl. No.: 10/091,752

(22) Filed: Mar. 5, 2002

Related U.S. Application Data
(63) Continuation-in-part of application No. 10/036,949, filed on Dec. 21, 2001.
Application No. 10/036,949 is a division of application No. 09/358,036, filed on Jul. 21, 1999, now Pat. No. 6,340,595.
Application No. 09/358,036 is a continuation-in-part of application No. 09/097,239, filed on Jun. 12, 1998, now Pat. No. 6,413,776.
(60) Provisional application No. 60/282,590, filed on Apr. 9, 2001.

(30) Foreign Application Priority Data
Jun. 8, 2001 (EP) 01870124.3
May 2, 2001 (EP) 01870095.5
Mar. 7, 2001 (EP) 01870038.5

(51) Int. Cl. C12Q 1/68; G01N 33/53; G01N 33/567; C12Q 1/66; C12N 15/861

(52) U.S. Cl. 435/6; 435/7.2; 435/456; 435/8

(57) ABSTRACT

The invention relates to the field of molecular genetics and medicine. In particular the present invention relates to the field of functional genomics. The present invention provides the methods and means for the identification of nucleic acids and the polypeptides encoded by these nucleic acids that have a function related to the E2F pathway, which were isolated in a high-throughput screening assay using the E2F transcription factor activity as a read-out. The identified compounds are suitable drug-targets to treat human diseases.

### CROSS-REFERENCE TO RELATED APPLICATIONS

This application is a continuation-in-part of U.S. patent application Ser. No. 10/03 6,949, filed on Dec. 21, 2001, which is a divisional of U.S. patent application Ser. No. 09/358,036, filed on Jul. 21, 1999, now U.S. Pat. No. 6,340,595, which is a continuation-in-part of pending U.S. patent application Ser. No. 09/097,239, filed on Jun. 12, 1998 and claims priority from European Application No. 0 1870124.3, filed on Jun. 8, 2001, European Application No. 0 1870095.5, filed on May 2, 2001, U.S. Provisional Application No. 60/282,590, filed on Apr. 9, 2001, and European Application No. 0 187003 8.5, filed on Mar. 7, 2001.

### FIELD OF THE INVENTION

The invention relates to high throughput methods for identifying the function of sample nucleic acids and their products.

The ultimate goal of the Human Genome Project is to sequence the entire human genome. The expected outcome of this effort is a precise map of the 70,000-100,000 genes that are expressed in man. Since the early 1980s, a large number of Expressed Sequence Tags (ESTs), which are partial DNA sequences read from the ends of complementary DNA (cDNA) molecules, have been obtained by both government and private research organizations. A hallmark of these endeavors, carried out by a collaboration between Washington University Genome Sequencing Center and members of the IMAGE (Integrated Molecular Analysis of Gene Expression) consortium (http:/www-bio.llnl.gov/bbrp/image/image.html), has been the rapid deposition of the sequences into the public domain and the concomitant availability of the sequence-tagged cDNA clones from several distributors (Marra, et al. (1998) Trends Genet. 14(1):4-7). At present, the collection of cDNAs is believed to represent approximately 50,000 different human genes expressed in a variety of tissues including liver, brain, spleen, B-cells, kidney, muscle, heart, alimentary tract, retina, and hypothalamus, and the number is growing daily.

Recent initiatives like that of the Cancer Genome Anatomy project support an effort to obtain full-length sequences of clones in the Unigene set (a set of cDNA clones that is publicly available). At the same time, commercial entities propose to validate 40,000 full-length cDNA clones. These individual clones will then be available to any interested party. The speed by which the coding sequences of novel genes are identified is in sharp contrast to the rate by which the function of these genes is elucidated. Assigning functions to the cDNAs in the databases, or functional genomics, is a major challenge in biotechnology today.

For decades, novel genes were identified as a result of research designed to explain a biological process or hereditary disease and the function of the gene preceded its identification. In functional genomics, coding sequences of genes are first cloned and sequenced and the sequences are then used to find functions. Although other organisms such as Drosophila, C. elegans, and Zebrafish are highly useful for the analysis of fundamental genes, animal model systems are inevitable for complex mammalian physiological traits (blood glucose, cardiovascular disease, inflammation). However, the slow rate of reproduction and the high housing costs of the animal models are a major limitation to high throughput functional analysis of genes. Although labor intensive efforts are made to establish libraries of mouse strains with chemically or genetically mutated genes in a search for phenotypes that allow the elucidation of gene function or that are related to human diseases, a systematic analysis of the complete spectrum of mammalian genes, be it human or animal, is a significant task.

In order to keep pace with the volume of sequence data, the field of functional genomics needs the ability to perform high throughput analysis of true gene function. Recently, a number of techniques have been developed that are designed to link tissue and cell specific gene expression to gene function. These include cDNA micro arraying and gene chip technology and differential display messenger RNA (mRNA). Serial Analysis of Gene Expression (SAGE) or differential display of mRNA can identify genes that are expressed in tumor tissue but are absent in the respective normal or healthy tissue. In this way, potential genes with regulatory functions can be separated from the excess of ubiquitously expressed genes that have a less likely chance to be useful for small drug screening or gene therapy projects. Gene chip technology has the potential to allow the monitoring of gene expression through the measurement of mRNA expression levels in cells of a large number of genes in only a few hours. Cells cultured under a variety of conditions can be analyzed for their mRNA expression patterns and compared to provide insight into their function and relationship to disease states.

One of the hallmarks of many disease states is the deregulation of the pRb tumor-suppressor pathway, either by mutation of pRb, its upstream regulator p16^{INK4a}, or by over expression of cyclin D, which associates with cyclin-dependent kinases (Cdks) that phosphorylate and thereby inactivate pRb (Weinberg, (1995) Cell 81:323-30). Besides the involvement of Rb in human cancers such as retinoblastoma and osteosarcoma, deregulation of the pRb pathway also underlies other human proliferative disorders such as the vascular disorders atherosclerosis and restenosis (Dzau, et al. (1996) Proc. Natl. Acad. Sci. USA 93:11421-5; Ishizaki, et al. (1996) Nat. Med. 2:1386-9). In either case, deregulation of the pRb pathway will result in the activation of the downstream components of the pathway: the E2F transcription factors.

The relevance of E2F transcription factors in the regulation of cell proliferation is underscored by the observation that over expression of E2F-1 in transgenic mice predisposes them to tumorigenesis (Pierce, et al. (1998) Oncogene 16:1267-76). In cell culture experiments, E2F-1 acts as a potent oncogene in transformation assays (Johnson, et al. (1994) Proc. Natl. Acad. Sci. USA 91:12823-7; Singh, et al. (1994) EMBO J. 13:3329-38). Furthermore, ectopic expression of E2F-1 is sufficient to drive quiescent cells into cell cycle (Johnson, et al. (1993) Nature 365:349-52).

In addition to its effect on proliferation, E2F also plays a critical role in the regulation of apoptosis. E2F-1 deficient mice develop a broad spectrum of tumors, suggesting that E2F may act as either an oncogene or a tumor suppressor, depending on the context in which activity is analysed (Yamasaki, et al. (1996) Cell 85:537-48). Increase of E2F expression following DNA damage also provides evidence that E2F can induce growth arrest and apoptosis (Sears and Nevins, (2002) J. Biol. Chem. in press; Blattner, et al. (1999) Mol. Cell. Biol. 19:3704-13). As proliferation and apoptosis are antagonistic processes, both activation and inhibition of E2F can result in either tumorigenesis or apoptosis, depending on the cellular context.

The E2F transcription factors are heterodimers containing a subunit encoded by the E2F gene family and a subunit encoded by the DP family of genes. To date six E2F genes (E2F-1 through 6) and two DP genes (DP-1 and DP-2) have been found in mammalian cells. E2F and DP proteins contain highly conserved DNA-binding and dimerization domains (Helin, (1998) Curr. Opin. Genet. Dev. 8:28-35). The carboxy-terminal portion of E2F 1-5 contains a potent transactivation domain, but no equivalent activity has been found in E2F-6 or in DP proteins. The different E2F heterodimers are regulated by interactions with members of the retinoblastoma gene family (pRb, p107 and p130). E2F1-3/DP complexes bind to pRb, E2F-4/DP heterodimers interact with pRb and p 107, and E2F-5 is preferentially bound by p 130. The association of E2Fs with pRb family members as well as their relative importance varies with specific stages of the cell cycle (Dyson, (1998) Genes Dev. 12:2245-62). In general, p130/E2F complexes are primarily found in quiescent or differentiated cells and p107/E2F complexes are most prevalent in S phase cells. pRb/E2F complexes can be found in quiescent or differentiated cells, but are most evident as cells progress from G1 into S phase. The progression through the mammalian cell cycle is cooperatively regulated by several classes of cyclin-dependent kinases (Cdks) and their regulatory subunits: the cyclins (reviewed in Sherr, (1994) Cell 79:551-5). The cyclins display a sequential appearance as cells move from quiescence (GO) into the first gap phase (G1), through initiation of DNA synthesis (S), and via the second gap phase (G2) to mitosis (M). The activity of Cdk complexes depends on their expression levels, association with cyclins, phosphorylation status and the association with specific Cdk-inhibitors (CKIs). The CKIs can be divided into two classes based on their structures and targets. The first class involves the INK4a family including p16^{INK4a}, p15^{INK4b}, p18^{INK4c} and p19^{INK4d} that act as inhibitors of D-type cyclins by inhibiting their catalytic partners: Cdk4 and Cdk6 (Hannon and Beach, (1994) Nature 371:257-61; Serrano, et al. (1993) Nature 366:704-7). The second class consists of the CIP/KIP proteins p21^{Cip1}, p27^{Kip1} and p57^{Kip2} whose actions regulate cyclin D-, cyclin E- and A-dependent kinases, by binding to both the cyclin and Cdk subunits (Harper, et al. (1993) Cell 75:805-16; Polyak, et al. (1994) Genes Dev. 8:9-22). When quiescent cells enter the cell cycle, activated cyclin D-dependent kinases trigger the phosphorylation of the retinoblastoma tumor-suppressor protein Rb, and the related family members p107 and p130 (Beijersbergen and Bemards, (1996) Biochim. Biophys. Acta 1287:103-20; Xiao, et al. (1996) Proc. Natl. Acad. Sci. USA 93:4633-7). Once pRb is primed with phosphates, Rb is further phosphorylated by cyclin E/Cdk2 complexes in late G1 phase (Lundberg and Weinberg, (1998) Mol. Cell. Biol. 18:753-61). The phosphorylation of the Rb family members results in the release and activation of the E2F/DP transcription factors, which play a central role in the control of cell proliferation. Inactivation of Rb, and subsequent activation of the E2F transcription factors at the G1/S boundary irreversibly commits the cells to complete the mitotic cycle (See FIG. 45 for schematic representation of Gi to S transition in the mammalian cell cycle).

Relatively little is known about the specific properties of the individual E2Fs but it is widely anticipated that different E2F heterodimers regulate various subsets of E2F target genes. E2F complexes bind to specific binding sites in the promoter regions of a number of cellular genes involved in DNA synthesis and regulation of the cell cycle, including DNA polymerase-α, dhfr, thymidine kinase, MCM genes, orcl, cdk2, cdc2, cdc6, cyclin A, cyclin E, c-myc and b-myb (reviewed in Muller and Helin, (2000) Biochim. Biophys. Acta 1470:M1-12). There appear to be three generic types of E2F complexes: activator E2F complexes, in which the E2F activation domain promotes transcription; inhibited E2F complexes, in which the activation domain is masked by pRb-family proteins to give a complex that is essentially inert; and repressor E2F complexes, in which Rb-family proteins that are recruited to the DNA by E2F, assemble a repressor activity. Apparently, the activation of E2F target genes may either result from transcriptional activation or loss of active repression on the promoter regions. As noted above, depending on the cellular context, this activation of E2F target genes can result in either proliferation or apoptosis.

The mechanism of E2F-mediated transcriptional activation remains unresolved. Possibly, E2F can regulate transcription via the recruitment of either TBP or CBP to E2F regulated promoters (Hagemeier, et al. (1993) Nucleic Acids Res. 21:4998-5004; Trouche, et al. (1996) Nucleic Acids Res. 24:4139-45). Also, although the Rb/E2F-mediated repression mechanism is unclear, a putative role for both HDACs and the SWI/SNF nucleosome-remodeling complexes in this mechanism has been suggested (Luo, et al. (1998) Cell 92:463-73; Trouche, et al. (1997) Proc. Natl. Acad. Sci. USA 94:11268-73). Thus, E2F binding sites serve to repress as well as to activate cellular promoters, depending on the nature of the E2F complexes found in the cell.

As uncontrolled cell proliferation underlies many different human diseases, disrupting the deregulated pathways may provide a good strategy to treat these proliferative disorders. Indeed, recent studies suggest that interfering with the INK4a/cyclinD/pRb/E2F pathway may prevent uncontrolled proliferation. For example, in vivo tumor suppression was observed in breast xenografts subsequent to the treatment of established tumors with an adenoviral vector expressing the pRb protein (Demers, et al. (1998) Cancer Gene Ther. 5:207-14). Furthermore, adenoviral mediated gene transfer of the retinoblastoma family proteins in a rat carotid artery model demonstrated that the inhibition of E2F activity resulted in reduced smooth muscle cell proliferation and prevented restenosis after angioplasty (Claudio, et al. (1999) Circ. Res. 85:1032-9). Also, it was shown with in vivo adenoviral gene therapy that directed over expression of the p16 gene efficiently inhibited the pathology in an animal model of rheumatoid arthritis (Taniguchi, et al. (1999) Nat Med 5:760-7). Moreover, ex-vivo gene therapy of human bypass grafts with E2F decoy oligodeoxynucleotides demonstrated that inhibition of E2F-mediated cell proliferation in these vein grafts lowered the failure rates of human primary bypass vein grafting (Mann, et al. (1999) Lancet 354:1493-8).

Conversely, as the activation of E2F-dependent transcription is also linked to apoptosis, therapeutic strategies may also take advantage of E2F-mediated cell death pathways. For instance, E2F can induce expression of p19^{ARF} (DeGregori, et al. (1997) Proc. Natl. Acad. Sci. USA 94:7245-50), which in turn promotes the accumulation of the p53 tumor suppressor (Prives, (1998) Cell 95:5-8; Sherr and Weber, (2000) Curr. Opin. Genet. Dev. 10:94-9). E2F is also a substrate for the kinase ATM, which is activated by DNA damage (Lin, et al. (2001) Genes Dev. 15: 1833-45). Phosphorylation of E2F blocks proteosome-mediated degradation of E2F, thus increasing E2F levels in the cell. In addition, phosphorylation of E2F itself may disrupt pRb/E2F complexes (Fagan, et al. (1994) Cell 78:799-811; Peeper, et al. (1995) Oncogene 10:39-48). Also, both the phosphorylation and acetylation of E2F have been reported to regulate E2F transactivation potential (Martinez-Balbas, et al. (2000) EMBO J. 19:662-71; Marzio, et al. (2000) J Biol Chem 275:10887-92; Morris, et al. (2000) Nat Cell Biol 2:232-9). Moreover, changing the subcellular localization of E2F complexes, which has been observed for E2F-4 containing complexes, may be a mechanism for regulating E2F activity (Muller, et al. (1997) Biochim. Biophys. Acta 1470:M1-12; Verona, et al. (1997) Mol. Cell. Biol. 17:7268-82). Furthermore, both the rate of E2F synthesis as well as ubiquitin-directed degradation will determine the amount of ‘free’ E2F in the cell (Hateboer, et al. (1996) Genes Dev. 10:2960-70; Hsiao, et al. (1994) Genes Dev. 8:1526-37; Sears, et al. (1997) Mol. Cell. Biol. 17:5227-35). Although pRb is the best-known regulator of E2F activity, the relative importance of the various suggested types of E2F regulation must be determined and new regulators may be identified. Clearly, those gene products that can alter E2F function are potential drug targets for proliferative disorders with deregulated E2F activity. However, since for most of the 40,000 genes a function still needs to be identified, there is a major hurdle to be taken to find those genes that act in the E2F pathway.

### Reported Developments

DNA microarray chips with 40,000 non-redundant human genes have been produced and were projected to be on the market in 1999 (Editorial, (1998) Nat. Genet. 18(3): 195-7). However, these techniques are primarily designed for screening cancer cells and not for screening for specific gene functions.

Double or triple hybrid systems also are used to add functional data to the genomic databases. These techniques assay for protein-protein, protein-RNA, or protein-DNA interactions in yeast or mammalian cells (Brent and Finley, (1997) Annu. Rev. Genet. 31:663-704). However, this technology does not provide a means to assay for a large number of other gene functions such as differentiation, motility, signal transduction, and enzyme and transport activity.

Yeast expression systems have been developed which are used to screen for naturally secreted and membrane proteins of mammalian origin (Klein, et al. (1996) Proc. Natl. Acad. Sci. USA 93(14):7108-13). This system also allows for collapsing of large libraries into libraries with certain characteristics that aid in the identification of specific genes and gene products. One disadvantage of this system is that genes encoding secreted proteins are primarily selected. A second disadvantage is that the library may be biased because the technology is based on yeast as a heterologous expression system and there will be gene products that are not appropriately folded.

The development of high throughput screens is discussed in Jayawickreme and Kost, (1997) Curr. Opin. Biotechnol. 8:629-634. A high throughput screen for rarely transcribed differentially expressed genes is described in von Stein, et al. (1997) Nucleic Acids Res. 35:2598-2602. High throughput genotyping is disclosed in Hall, et al. (1996) Genome Res. 6:781-790. Methods for screening transdominant intracellular effector peptides and RNA molecules are disclosed in Nolan, WO 97/27212 and WO 97/27213.

Other current strategies include the creation of transgenic mice or knockout mice. A successful example of gene discovery by such an approach is the identification of the osteoprotegerin gene. DNA databases were screened to select ESTs with features suggesting that the cognate genes encoded secreted proteins. The biological functions of the genes were assessed by placing the corresponding full-length cDNAs under the control of a liver-specific promoter. Transgenic mice created with each of these constructs consequently have high plasma levels of the relevant protein. Subsequently, the transgenic animals were subjected to a battery of qualitative and quantitative phenotypic investigations. One of the genes that was transfected into mice produced mice with an increased bone density, which led subsequently to the discovery of a potent anti-osteoporosis factor (Simonet, et al. (1997) Cell 89(2):309-19). The disadvantages of this method are that the method is costly and highly time consuming.

The challenge in functional genomics is to develop and refine all the above-described techniques and integrate their results with existing data in a well-developed database that provides for the development of a picture of how gene function constitutes cellular metabolism and a means for this knowledge to be put to use in the development of novel medicinal products. The current technologies have limitations and do not necessarily result in true functional data. Therefore, there is a need for a method that allows for direct measurement of the function of a single gene from a collection of genes (gene pools or individual clones) in a high throughput setting in appropriate in vitro assay systems and animal models. A method for identifying genes having proliferative- or apoptotic-related function(s) from a large array of gene sequences has not been reported.

### SUMMARY OF THE INVENTION

The present invention relates to methods, and compositions for use therein, for identifying, in a high throughput setting, unique nucleic acids involved in apoptosis-associated processes in cells using libraries of vectors comprising such nucleic acids. More particularly, the present invention relates to a method of identifying a unique nucleic acid capable of altering E2F activity in a cell, wherein said unique nucleic acid is present in a library, said method comprising: (a) providing a library of a multitude of unique expressible nucleic acids, said library including a multiplicity of compartments, each of said compartments consisting essentially of one or more adenoviral vector comprising at least one unique nucleic acid of said library in an aqueous medium, wherein said adenoviral vector is capable of introducing said nucleic acid into a host cell, is capable of expressing the product of said nucleic acid in said host cell, and is deleted in a portion of the adenoviral genome necessary for replication thereof in said host cell; (b) transducing a multiplicity of host cells with at least one adenoviral vector comprising at least one unique nucleic acid from said library; (c) incubating said host cells to allow expression of the product of said nucleic acid; and (d) determining if E2F activity is altered in said cell. The host cell transduced with said recombinant adenoviral vector is observed for a change in E2F activity, and if such activity change is identified, an apoptosis-associated function is assigned to the product(s) encoded by the sample nucleic acids.

The present method also comprises: (a) growing a plurality of cell cultures containing at least one cell, said one cell expressing adenoviral sequence consisting essentially of E1-region sequences and expressing one or more functional gene products encoded by at least one adenoviral region selected from an E2A region and an E4 region; (b) transfecting, under conditions whereby said recombinant adenovirus vector library is produced, said at least one cell in each of said plurality of cell cultures with

i) an adapter plasmid comprising adenoviral sequence coding, in operable configuration, for a functional Inverted Terminal Repeat, a functional encapsidation signal, and sequences sufficient to allow for homologous recombination with a first recombinant nucleic acid, and not coding for E1 region sequences which overlap with E1 region sequences in said at least one cell, for E1 region sequences which overlap with E1 region sequences in a first recombinant nucleic acid, for E2B region sequences other than essential E2B sequences, for E2A region sequences, for E3 region sequences and for E4 region sequences, and further comprises a unique nucleic acid sequence and promoter operatively linked to said unique nucleic acid sequence; and

ii) a first recombinant nucleic acid comprising adenoviral sequence coding, in operable configuration, for a functional adenoviral Inverted Terminal Repeat and for sequences sufficient for replication in said at least one cell, but not comprising adenoviral E1 region sequences which overlap with E1 sequences in said at least one cell, and not comprising E2A region sequences or E4 region sequences expressed in said plurality of cells which would otherwise lead to production of replication competent adenovirus wherein said first recombinant nucleic acid has sufficient overlap with said adapter plasmid to provide for homologous recombination resulting in production of recombinant adenoviral vectors in said at least one cell;

(c) incubating said plurality of cells under conditions which result in the lysis of said plurality of cells facilitating the release of said recombinant adenoviral vectors containing said unique nucleic acid; (d) transferring an aliquot of said adenoviral vectors into a corresponding plurality of host cell cultures consisting of cells in which said vectors do not replicate, but in which said nucleic acids are expressible; (e) incubating said host cells to allow expression of the product of said nucleic acid; and (f) observing said host cell for a change in E2F activity.

A further aspect of the present assay methods is determining whether the expression product of the nucleic acid capable of altering E2F activity is secreted by said cell, comprising: (a) infecting producer cells in a medium with an adenoviral vector comprising a unique nucleic acid capable of altering E2F activity; (b) combining said medium with test cells that have not been infected with said vector; and (c) determining if E2F activity is altered in said test cells.

Another aspect of the present invention relates to a method for identifying a drug candidate compound useful in the treatment of a disease state related to E2F-disregulation, said method comprising: (a) contacting a first subpopulation of host cells transfected with polynucleotide, identified in the above-described method of the invention, with one or more of said test compound, and (b) identifying, from said one or more test compounds, a candidate compound that alters E2F activity in said first subpopulation of transfected host cells relative to a second subpopulation of said transfected host cells that have not been contacted with said test compound.

Another means of detecting candidate compounds comprises selecting a compound that induces either an increase or decrease in the expression of mRNA encoded by a polynucleotide comprising a sequence of SEQ ID NO: 13 in said first subpopulation of transfected host cells relative to the expression of said mRNA in a second subpopulation of transfected host cells that has not been contacted with such compound.

A further aspect of the present method comprises first determining the binding affinity of said one or more test compound to (1) the polynucleotide identified in accordance with the present methods invention, or (2) the corresponding antisense sequences thereof, or (3) an expression product of said sequences, by contacting one or more test compound therewith.

The present method is useful for identifying compounds that are suitable as drug candidate compounds, the pharmaceutical application of which is related to whether the aforesaid assay results in either an increase or a decrease in E2F activity, or the mRNA expression of the above-identified polynucleotides, in the host cells. If a test compound alters E2F activity, then the compound is useful for the treatment of apoptosis-associated disorders.

The present invention also relates to pharmaceutical compositions and methods of treatment comprising the polypeptides or polynucleotides described hereinbelow. Other aspects and more detailed description of the present invention are provided in the following sections.

### BRIEF DESCRIPTION OF THE DRAWINGS

FIG. 1: Construction of pBS.PGK.PCRI. pBS.PGK.PCRI encodes the human phosphoglycerate kinase (PGK) promoter operatively linked to adenovirus 5 (Ad5) E1 nucleotides 459-916. To construct this plasmid, Ad5 nucleotides 459-916 are amplified by the polymerase chain reaction (PCR) with primers Ea-1 (SEQ ID NO: 1) and Ea-2 (SEQ ID NO:2), digested with Cla I, and cloned into the ClaI-EcoRV sites of pBluescript (Stratagene), resulting in pBS.PCRI. The PGK promoter is excised from pTN by complete digestion with ScaI and partial digestion with EcoRI and cloned into the corresponding sites of pBS.PCRI, resulting in pBS.PGK.PCRI.

FIG. 2: Construction of pIG.E1A.E1B.X. pIG.E1A.E1B.X encodes Ad5 nucleotides 459-5788 (E1A and E1B regions) operatively linked to the human PGK promoter. pIG.E1A.E1B.X also encodes Ad5 pIX protein. pIG.E1A.E1B.X is constructed by replacing the ScaI-BspEI fragment of pAT-X/S with the corresponding fragment of pBS.PGK.PCRI.

FIG. 3A: Construction of pAT-PCR2-NEO. To construct this plasmid, the E1B promoter and initiation codon (ATG) of the E1B 21 kDa protein are PCR amplified with primers Ea-3 (SEQ ID NO:3) and Ep-2 (SEQ ID NO:4), where Ep-2 introduces an NcoI site (5′-CCATGG) at the 21 kDa protein initiation codon. The PCR product (PCRII) is digested with HpaI and NcoI and ligated into the corresponding sites of pAT-X/S, producing pAT-X/S-PCR2. The NcoI-StuI fragment of pTN, containing the Neo^{R} and a portion of the HBV poly(A) site is ligated into the NcoI-NruI sites of pAT-X/S-PCR2, producing pAT-PCR2-NEO.

FIG. 3B: Construction of pIG.E1A.NEO. pIG.E1A.NEO encodes Ad5 nucleotides 459-1713 operatively linked to the human PGK promoter. Also encoded is the E1B promoter functionally linked to the neomycin resistance gene (Neo^{R}) and the hepatitis B virus (HBV) poly(A) signal. In this construct, the AUG codon of the E1B 21 kDa protein functions as the initiation codon of Neo^{R}. The HBV poly(A) signal of pAT-PCR2-NEO (see FIG. 3A) is completed by replacing the ScaI-SalI fragment of pAT-PCR2-NEO with the corresponding fragment of pTN, producing pAT.PCR2.NEO.p(A), and replacing the ScaI-XbaI fragment of pAT.PCR2.NEO.p(A) with the corresponding fragment of pIG.E1A.E1B.X, producing pIG.E1A.NEO.

FIG. 4: Construction of pIG.E1A.E1B. pIG.E1A.E1B contains the Ad5 nucleotides 459-3510 (E1A and E1B proteins) operatively linked to the PGK promoter and HBV poly(A) signal. This plasmid is constructed by PCR amplification of the N-terminal amino acids of the E1B 55 kDa protein with primers Eb-1 (SEQ ID NO:5) and Eb-2 (SEQ ID NO:6), which introduces an XhoI site, digested with BglII and cloned into the BglII-NruI sites of pAT-X/S, producing pAT-PCR3. The XbaI-XhoI fragment of pAT-PCR3 is replaced with the XbaI-SalI fragment (containing the HBV poly(A) site) of pIG.E1A.NEO to produce pIG.E1A.E1B.

FIG. 5: Construction of pIG.NEO. pIG.NEO contains the Neo^{R} operatively linked to the E1B promoter. pIG.NEO was constructed by ligating the HpaI-ScaI fragment of pAT.PCR2.NEO.p(A) or pIG.E1A.NEO, which contains the E1B promoter and Neo^{R} into the EcoRV-ScaI sites of pBS.

FIG. 6: Transformation of primary baby rat kidney (BRK) cells by adenoviral packaging constructs. Subconfluent dishes of BRK cells are transfected with 1 or 5 μg of either pIG.NEO, pIG.E1A.NEO, pIG.E1A.E1B, pIG.E1A.E1B.X, pAd5XhoIC, or pIG.E1A.NEO plus pDC26, which expresses the Ad5 E1B gene under control of the SV40 early promoter. Three weeks post-transfection, foci are visible, cells are fixed, Giemsa stained and the foci counted. The results shown are the average number of foci per 5 replicate dishes.

FIG. 7: Western blot analysis of A549 clones transfected with pIG.E1A.NEO and human embryonic retinoblasts (HER) cells transfected with pIG.E1A.E1B (PER clones). Expression of Ad5 E1A and E1B 55 kDa and 21 kDa proteins in transfected A549 cells and PER cells is determined by Western blot with mouse monoclonal antibodies (Mab) M73, which recognizes E1A gene products, and Mabs AIC6 and CIG11, which recognize the E1B 55 kDa and 21 kDa proteins, respectively. Mab binding is visualized using horseradish peroxidase-labelled goat anti-mouse antibody and enhanced chemiluminescence. 293 and 911 cells serve as controls.

FIG. 8: Southern blot analysis of 293, 911 and PER cell lines. Cellular DNA is extracted, HindIII digested, electrophoresed, and transferred to Hybond N+ membranes (Amersham). Membranes are hybridized to radiolabelled probes generated by random priming of the SspI-HindIII fragment of pAd5.SalB (Ad5 nucleotides 342-2805).

FIG. 9: Transfection efficiency of PER.C3, PER.C5, PER.C6 and 911 cells. Cells are cultured in 6-well plates and transfected in duplicate with 5 μg pRSV.lacZ by calcium-phosphate co-precipitation. Forty-eight hours post-transfection, cells are stained with X-Gal, and blue cells are counted. Results shown are the mean percentage of blue cells per well.

FIG. 10: Construction of adenoviral vector, pMLPI.TK. pMLPI.TK is designed to have no sequence overlap with the packaging construct pIG.E1A.E1B. pMLPI.TK is derived from pMLP.TK by deletion of the region of sequence overlap with pIG.E1A.E1B and deletion of non-coding sequences derived from lacZ. SV40 poly(A) sequences of pMLP.TK are PCR amplified with primers SV40-1 (SEQ ID NO:7), which introduces a BamHI site, and SV40-2 (SEQ ID NO:8), which introduces a BglII site. pMLP.TK Ad5 sequences 2496 to 2779 are PCR amplified with primers Ad5-1 (SEQ ID NO:9), which introduces a BglII site, and Ad5-2 (SEQ ID NO:10). Both PCR products are BglII digested, ligated, and PCR amplified with primers SV40-1 and Ad5-2. This third PCR product is BamHI and AflIII digested and ligated into the corresponding sites of pMLP.TK, producing pMLPI.TK.

FIG. 11A: New adenoviral packaging construct, pIG.E1A.E1B, does not have sequence overlap with new adenoviral vector, pMLPI.TK. Regions of sequence overlap between the packaging construct pAd5XhoIC, expressed in 911 cells, and adenoviral vector pMLP.TK, that can result in homologous recombination and the formation of RCA are shown. In contrast, there are no regions of sequence overlap between the new packaging construct pIG.E1A.E1B, expressed in PER.C6 cells, and the new adenoviral vector pMLPI.TK.

FIG. 11B: New adenoviral packaging construct pIG.E1A.NEO, does not have sequence overlap with new adenoviral vector pMLPI.TK. There are no regions of sequence overlap between the new packaging construct pIG.E1A.NEO and the new adenoviral vector pMLPI.TK that can result in homologous recombination and the formation of RCA.

FIG. 12: Generation of recombinant adenovirus, IG.Ad.MLPI.TK. Recombinant adenovirus IG.Ad.MLPI.TK is generated by co-transfection of 293 cells with SalI linearized pMLPI.TK and the right arm of ClaI digested, wild-type Ad5 DNA. Homologous recombination between linearized pMLPI.TK and wild-type Ad5 DNA produces IG.Ad.MLPI.TK DNA, which contains an E1 deletion of nucleotides 459-3510. 293 cells transcomplement the deleted Ad5 genome, thereby permitting replication of the IG.Ad.MLPI.TK DNA and its packaging into virus particles.

FIG. 13: Rationale for the design of adenoviral-derived recombinant DNA molecules that duplicate and replicate in cells expressing adenoviral replication proteins. A diagram of the adenoviral double-stranded DNA genome indicating the approximate locations of E1, E2, E3, E4, and L regions is shown. The terminal polypeptide (TP) attached to the 5′-terminus is indicated by closed circles. The right arm of the adenoviral genome can be purified by removal of the left arm by restriction enzyme digestion. Following transfection of the right arm into 293 or 911 cells, adenoviral DNA polymerase (white arrow) encoded on the right arm will produce only single-stranded forms. Neither the double-stranded nor single-stranded DNA can replicate because they lack an inverted terminal repeat (ITR) at one terminus. Providing the single-stranded DNA with a sequence that can form a hairpin structure at the 3′-terminus, which serves as a substrate for DNA polymerase, will extend the hairpin structure along the entire length of the molecule. This molecule can also serve as a substrate for a DNA polymerase, but the product is a duplicated molecule with ITRs at both termini that can replicate in the presence of adenoviral proteins.

FIG. 14: Adenoviral genome replication. The adenoviral genome is shown in the top left panel. The origins or replication are located within the left and right ITRs at the genome ends. DNA replication occurs in two stages. Replication proceeds from one ITR, generating a daughter duplex and a displaced parental single-strand that is coated with adenoviral DNA binding protein (DBP, open circles) and can form a panhandle structure by annealing of the ITR sequences at both termini. The panhandle is a substrate for DNA polymerase (Pol: white arrows) to produce double-stranded genomic DNA. Alternatively, replication proceeds from both ITRs, generating two daughter molecules, thereby obviating the requirement for a panhandle structure.

FIG. 15: Potential hairpin conformation of a single-stranded DNA molecule that contains the HP/asp sequence (SEQ ID NO: 11). Asp718I digestion of pICLha, containing the cloned oligonucleotides HP/asp1 and HP/asp2, yields a linear double-stranded DNA with an Ad5 ITR at one terminus and the HP/asp sequence at the other terminus. In cells expressing the adenoviral E2 region, a single-stranded DNA is produced with an Ad5 ITR at the 5′-terminus and the hairpin conformation at the 3′-terminus. Once formed, the hairpin can serve as a primer for cellular and/or adenoviral DNA polymerase to convert the single stranded DNA to double stranded DNA.

FIG. 16: Diagram of pICLhac. pICLhac contains all the elements of pICL (FIG. 19) but also contains the HP/asp sequence in the Asp718 site in an orientation that will produce the hairpin structure shown in FIG. 15, following linearization by Asp718 digestion and transfection into cells expressing adenoviral E2 proteins.

FIG. 17: Diagram of pICLhaw. pICLhaw is identical to pICLhac (FIG. 16) except that the inserted HP/asp sequence is in the opposite orientation.

FIG. 18: Schematic representation of pICLI. pICLI contains all the elements of pICL (FIG. 19) but also contains an Ad5 ITR in the Asp718 site.

FIG. 19: Diagram of pICL. pICL is derived from the following: (i) nucleotides 1-457, Ad5 nucleotides 1-457 including the left ITR, (ii) nucleotides 458-969, human Cytomegalovirus (CMV) enhancer and immediate early promoter, (iii) nucleotides 970-1204, SV40 19S exon and truncated 16/19S intron, (iv) nucleotides 1218-2987, firefly luciferase gene, (v) nucleotides 3018-3131, SV40 tandem polyadenylation signals from the late transcript, (vi) nucleotides 3132-5620, pUC12 sequences including an Asp718 site, and (vii) ampicillin resistance gene in reverse orientation.

FIG. 20: Shows a schematic overview of the adenoviral fragments cloned in pBr322 (plasmid) or pWE15 (cosmid) derived vectors. The top line depicts the complete adenoviral genome flanked by its ITRs (filled rectangles) and with some restriction sites indicated. Numbers following restriction sites indicate approximate digestion sites (in kb) in the Ad5 genome.

FIG. 21: Drawing of adapter plasmid pAd/L420-HSA

FIG. 22: Drawing of adapter plasmid pAd/Clip

FIG. 23: Schematic representation of the generation of recombinant adenoviruses using a plasmid-based system. In the top of the figure, the genome organization of Ad5 is shown with filled boxes representing the different early and late transcription regions and flanking ITRs. The middle of the figure represents the two DNAs used for a single homologous recombination while the bottom of the figure represents the recombinant virus after transfection into packaging cells.

FIG. 24: Drawing of minimal adenoviral vector pMV/L420H

FIG. 25: Helper construct for replication and packaging of minimal adenoviral vectors. Schematic representation of the cloning steps for the generation of the helper construct pWE/AdΔ5′.

FIG. 26: Evidence for SV40-LargeT/ori mediated replication of large adenoviral constructs in COS-1 cells. FIG. 26A shows a schematic representation of construct pWE/Ad. Δ5′. The location of the SV40 ori sequence and the fragments used to prepare probes are indicated. Evidence for SV40-LargeT/ori mediated replication of large adenoviral constructs in COS-1 cells. FIG. 26B shows an autoradiogram of the Southern blot hybridized to the adenoviral probe. FIG. 26C shows an autoradiogram of the Southern blot hybridized to the pWE probe. Lane 1, marker lane: λ DNA digested with EcoRI and HindIII. Lane 4 is empty. Lanes 2, 5, 7, 9, 11, 13, 15, and 17 contain undigested DNA and Lanes 3, 6, 8, 10, 12, 14, 16 and 18 contain MboI digested DNA. All lanes contain DNA from COS-1 cells transfected with pWE.pac (lanes 2 and 3), pWE/Ad.Δ5′ construct #1 (lanes 5 and 6), #5 (lanes 7 and 8) and #9 (lanes 9 and 10), pWE/Ad.ΔflII-rITR (lanes 11 and 12), pMV/CMV-LacZ (lanes 13 and 14), pWE.pac digested with PacI (lanes 15 and 16), or pWE/Ad.AflIII-rITR digested with PacI (lanes 17 and 18) as described in the text. Arrows point to the expected positive signal of 1416 bp (FIG. 26B) and 887 bp (FIG. 26C).

FIG. 27: Production of E1/E2A deleted adenoviral vectors and its efficiency in miniaturized PER.C6/E2A based production system.

FIG. 28: Average titers produced in a 96-well plate as measured using a PER.C6/E2A based plaque assay.

FIG. 29: Fidelity of adenoviral vector production miniaturized PER.C6/E2A based production system for a number of marker and human cDNA transgenes.

FIG. 30: Percentage of wells showing CPE formation after transfection of PER. C6/E2A cells with pCLIP-LacZ, purified by 6 different protocols. Qiagen: standard alkaline lysis followed by Qiagen plasmid purification; AlkLys: alkaline lysis followed by isopropanol precipitation, and solubilization in TE buffer; AL+RNase: alkaline lysis followed by isopropanol precipitation, and solubilization in TE buffer containing RNase at 10 microgram per ml; AL+R+phenol: alkaline lysis followed by isopropanol precipitation, and solubilization in TE buffer containing RNase at 10 microgram per ml, followed by phenol/chloroform extraction and ethanol precipitation; cetyltrimethylammoniumbromide (CTAB): Standard CTAB plasmid isolation; CTAB+phenol: Standard CTAB plasmid isolation, but solubilization in TE buffer containing RNase at 10 microgram per ml, followed by phenol/chloroform extraction.

FIG. 31: Effect of using digested plasmid for transfection without phenol-chloroform extraction. The results of all experiments are depicted and expressed as percentage of wells showing CPE formation. A) LacZ-adapter DNA is isolated using 6 different isolation methods; 1: Qiagen, 2: Alkaline lysis, 3: Alkaline lysis+RNase treatment, 4: Alkaline lysis+RNase treatment+p/c purification of DNA before linearization, 5: cetyltrimethylammoniumbromide (CTAB), 6: CTAB+p/c purification of DNA before linearization, rITR is p/c purified, B) Purified and unpurified EGFP- and EYFP-adapter DNA, rITR is p/c purified, C) EGFP-adapter DNA and rITR are tested purified and unpurified; 1: Both adapter and rITR purified (control), 2: rITR purified, adapter DNA unpurified, 3: rITR and adapter unpurified.

FIG. 32: Stability of adenoviral vectors produced in miniaturized format and incubated for up to three weeks at three different temperatures and measured using a plaque forming assay for adenoviral vectors.

FIG. 33: Efficiencies of virus generation in percentages of CPE after virus generation of several adenoviruses (E1 and E2A deleted) containing cDNAs in antisense (AS) orientation.

FIGS. 34A-M: Plasmid maps of adenoviral adapter plasmids. These adenoviral adapter plasmids are particularly useful for the construction of expression libraries in adenoviral vectors such as the subject of this application. They have very rare restriction sites for the linearization of adapters and libraries of adapters (with transgenes inserted) and will not inactivate the adapter by digestion of the inserts. In FIG. 34M, the cosmid containing pIPspAdapt5- or pCLIP-Ippol-polynew-derived adenoviral DNA can be used for in vitro ligations. Double stranded oligonucleotides containing compatible overhangs are ligated between the I-CeuI and PI-SceI sites, between I-CeuI and I-PpoI, between I-SceI and PI-SceI, and between I-SceI and I-PpoI. The PacI restriction endonuclease is subsequently used not only to linearize the construct after ligation and liberate the left- and right ITRs, but also to eliminate non-recombinants.

FIG. 34N: Percentage of wells showing CPE formation after transfection of PER.C6/E2A cells with pCLIP-LacZ and the adapter plasmid pIPspAdapt2.

FIG. 35: Percentage of virus producing wells (CPE positive) in a 96-well plate of PER.C6/E2A cell after propagation of the freeze/thawed transfected cell lysates. Helper molecules used for cotransfection are (1) pWE/Ad.AflII-rITRsp, (2) pWE/Ad.AflII-rITRsp.dE2A, (3) pWE/Ad.AflII-rITRsp.dXba, and (4) pWE/Ad.AflII-rITR.

FIGS. 36(A and B): Schematic overview of constructing an arrayed adenoviral cDNA expression library.

FIGS. 37(A, B, C, and D): Comparison of cotransfections of different adapter plasmids and pWE/Ad.AflII-rITRDE2A on 384-well plates with cotransfections on 96-well plates. The percentage of virus producing wells (CPE positive wells) scored at different time points as indicated after propagation of the freeze/thawed transfected cells to new PER.C6/E2A cells 5 days after transfection (upper panel) or 7 days after transfection (lower panel) is shown.

FIG. 38: The percentage of virus producing wells (CPE positive wells) scored at different time points as indicated after changing the medium of the transfected cells 7 days after transfection (A); after no medium change (B); and after standard propagation of the freeze/thawed transfected cells to new PER.C6/E2A cells (C).

FIGS. 39(A, B, and C): The percentage of virus producing cells (CPE positive) scored after propagation of the freeze/thawed transfected cells to new PER.C6/E2A cells, in three different experiments using PER.C6/E2A cells for transfections with indicated confluence at time of transfection. Cell numbers from each flask in each experiment were counted. The cells from these flasks were used to seed 96-well plates for transfection with adenoviral adapter and helper DNA molecules.

FIG. 40: The use of adenoviral expression vectors as a semi-stable expression system for assays with a delayed readout of phenotype after infection with an adenoviral expression library. Transgene used: Green Fluorescent Protein (EGFP, Clontech). A crude PER.C6/E2A production lysate is used at a multiplicity of infection (MOI) of about 500-1000.

FIG. 41: The use of polyethylenimine (PEI) for generating adenoviral vectors in miniaturized format. Transfection efficiency, virus formation (CPE), and proliferation (toxicity) are depicted.

FIG. 42: Effect of temperature PEI at time of transfections on CPE efficiency. W: Warm (room temperature) and C: Cold (4° C.).

FIG. 43: Effect of PEI transfection volume on transfection efficiencies.

FIG. 44: Washing of PER.C6/E2A cells with serum free medium before applying lipofectamine-DNA complex can be omitted.

FIG. 45: Progression from GI to S phase in the mammalian cell cycle.

FIG. 46: Schematic representation of the construction of adenoviral Placenta library.

FIG. 47: Schematic representation of pGL3-TATA-6xE2F-luc. The E2F binding sites are depicted as arrows over SEQ ID NO: 12.

FIG. 48: Schematic representation of pIPspAdapt8-L61Ras.

FIG. 49: Schematic representation of pIpSpAdApt3-E2F2.

FIG. 50: Schematic representation of pIpSpAdApt3-E2F3.

FIG. 51: Schematic representation of pIpSpAdApt6-p16^{INK}.

FIG. 52: Schematic representation of pIpSpAdApt6-p27^{KIP}.

FIG. 53: Schematic representation of pIpSpAdApt6-EGFP.

FIG. 54: Schematic representation of pCLIPPac-L61Ras.

FIG. 55: Schematic representation of pIpSpAdApt6-LacZ.

FIG. 56: Schematic representation of the various E2F reporter cell lines tested+controls.

FIG. 57: Schematic representation of the optimalization infection conditions E2F-reporter cell line IC5. Assay at different MOI.

FIG. 58: Schematic representation of the optimization of infection conditions E2F reporter cell line IC5. Assay at 48 or 72 hours after infection.

FIG. 59: Schematic representation of the optimization of infection conditions E2F reporter cell line IC5. High/Low serum conditions.

FIG. 60: Schematic representation of rescreen: reporter assay on cell line IC5 with first hits from 1500 screen.

FIG. 61: Schematic representation validation (transient reporter) of hits from rescreen (1500).

FIG. 62: Schematic representation of reporter assay in 384-wells format with control viruses from control virus plate.

FIG. 63: Schematic representation of the performance of control viruses that were implemented in the 11,000 library virus reporter screen.

FIG. 64: Schematic representation of the results obtained for 51 hits in the first screen and rescreen at approximate MOIs of 600 and 2000.

FIG. 65: Comparison of the results of the hits obtained in first 11,000 screen and retested in rescreen.

FIG. 66: Schematic representation validation (transient reporter) of hits from rescreen (11,000). A: E2F reporter, B: control reporter.

FIG. 67: Nucleotide (FIG. 67A) and deduced amino acid sequences (FIG. 67B) of Hit H1-9.

FIG. 68: H1-9 induces E2F activity in transient reporter assay. U2OS cells were transiently transfected with E2F-luciferase (marked as (E2F)6) or pGL3 (marked as control) together with increasing amounts (0, 0.5, 2.5 μg) of different effector plasmids (E2F2, H1-9, EGFP) and pRL-CMV as internal standard. The cells were harvested 40 hrs post-transfection and relative luciferase over Renilla values were measured and plotted.

FIG. 69: Optimization virus ratios for co-infections on U2OSwt cells.

FIG. 70: Optimization virus ratios for co-infections on U2OSwt cells.

FIG. 71: Fill up experiment on HUVEC cells by co-infections with increasing amounts of empty virus.

FIG. 72: Co-infection of HUVEC cells with viruses from the placenta library.

FIG. 73: Co-infection of HUVEC cells with viruses from the placenta library.

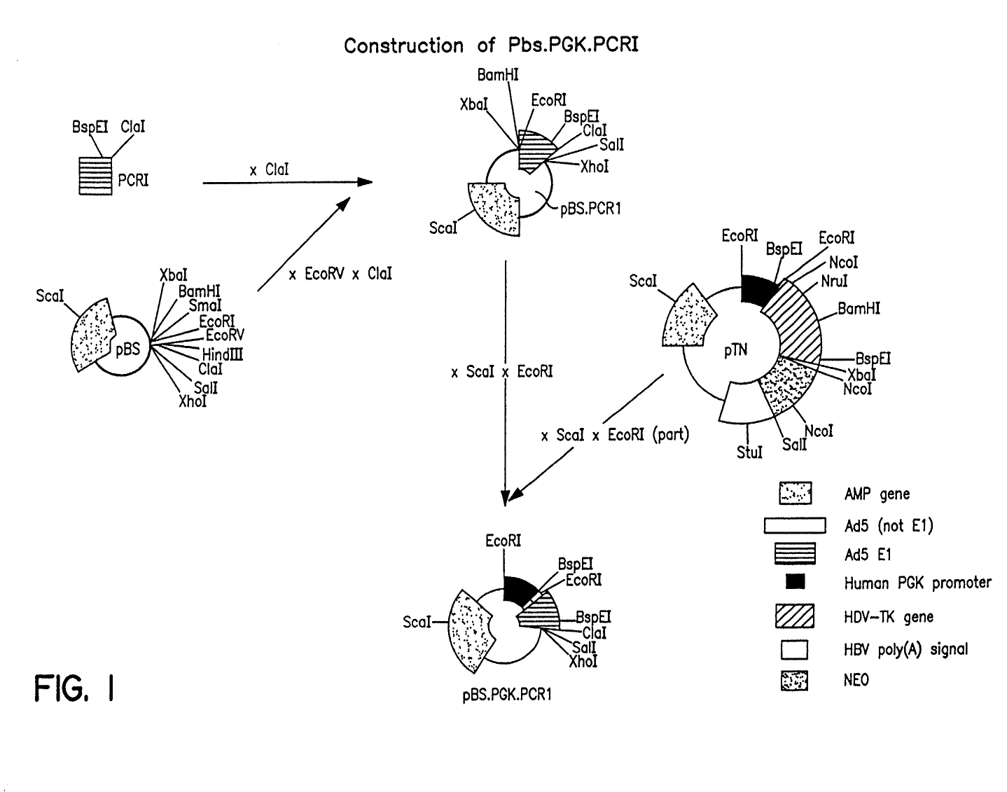

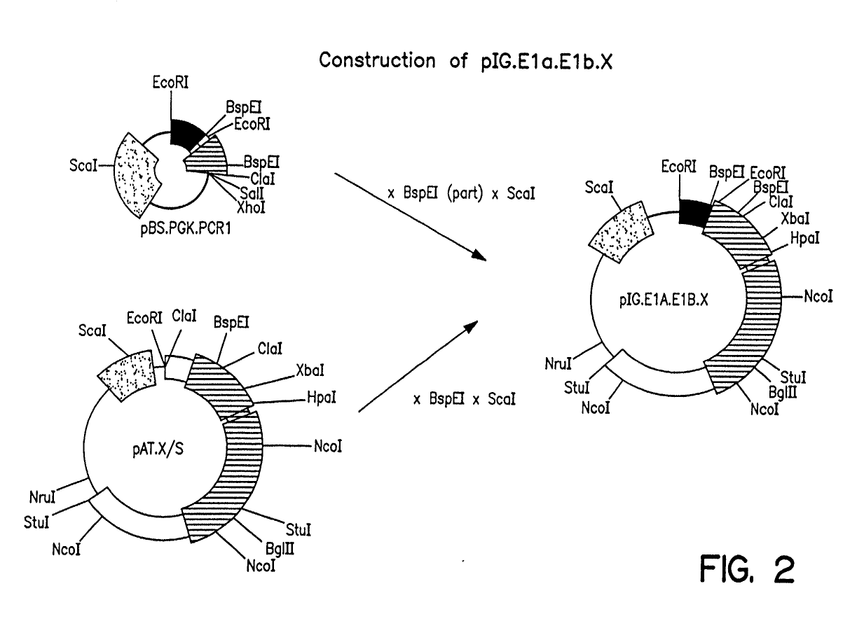

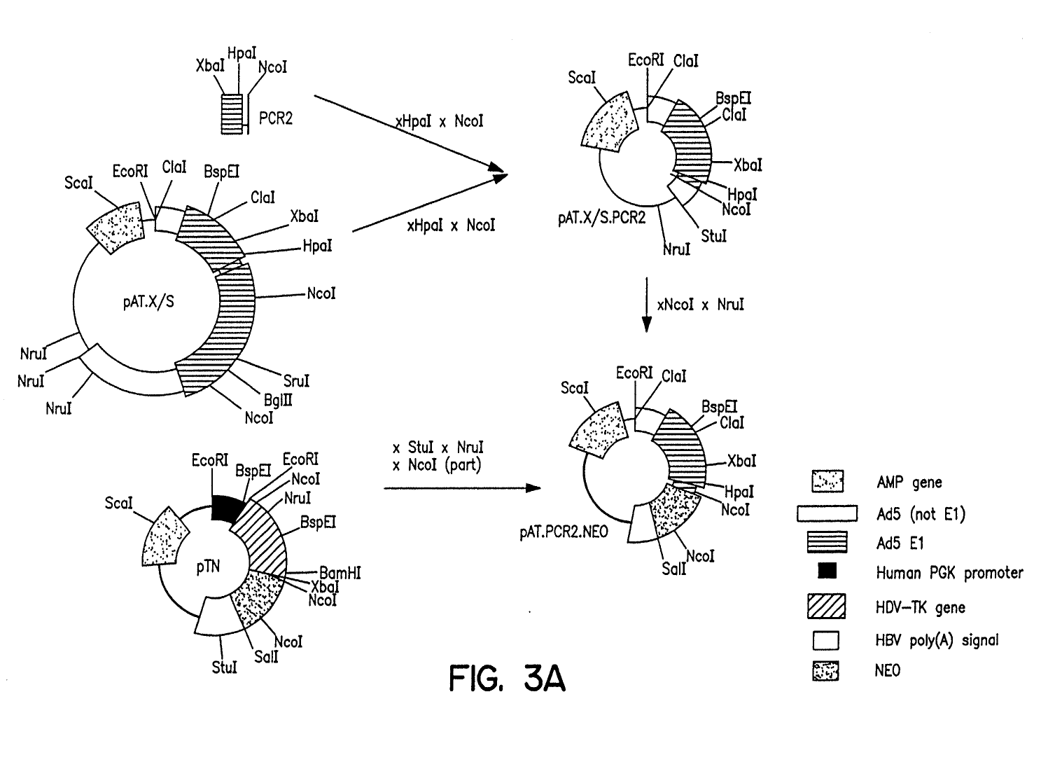

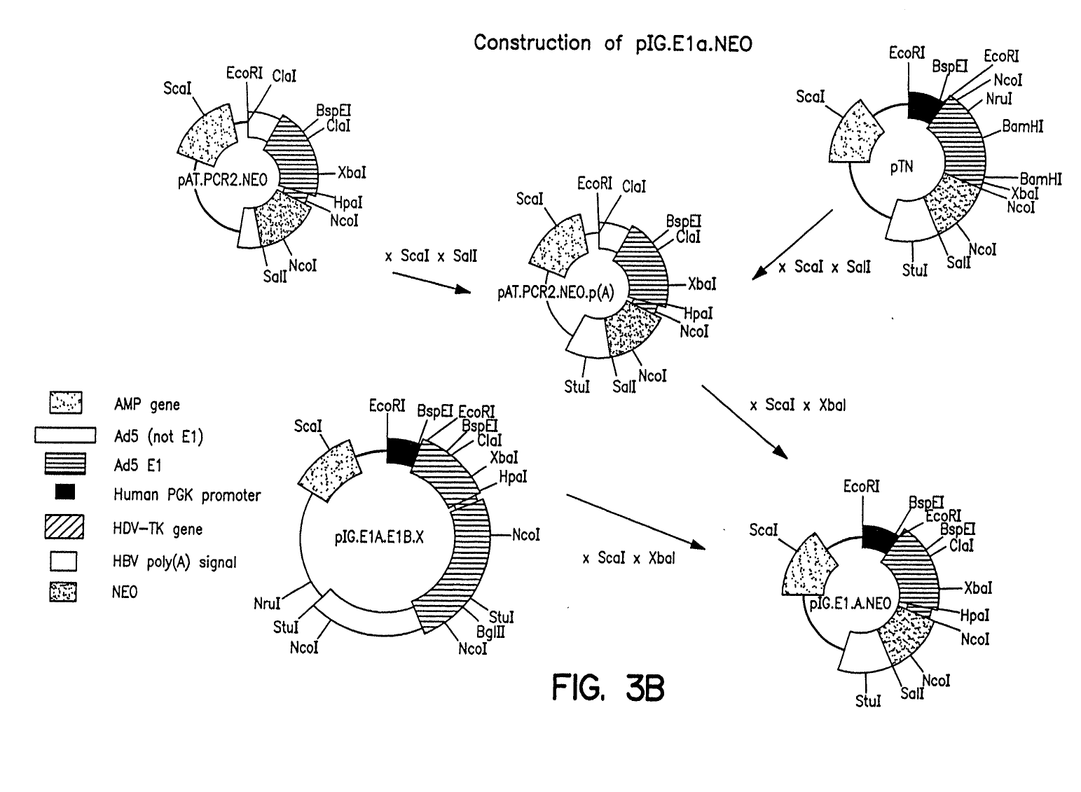

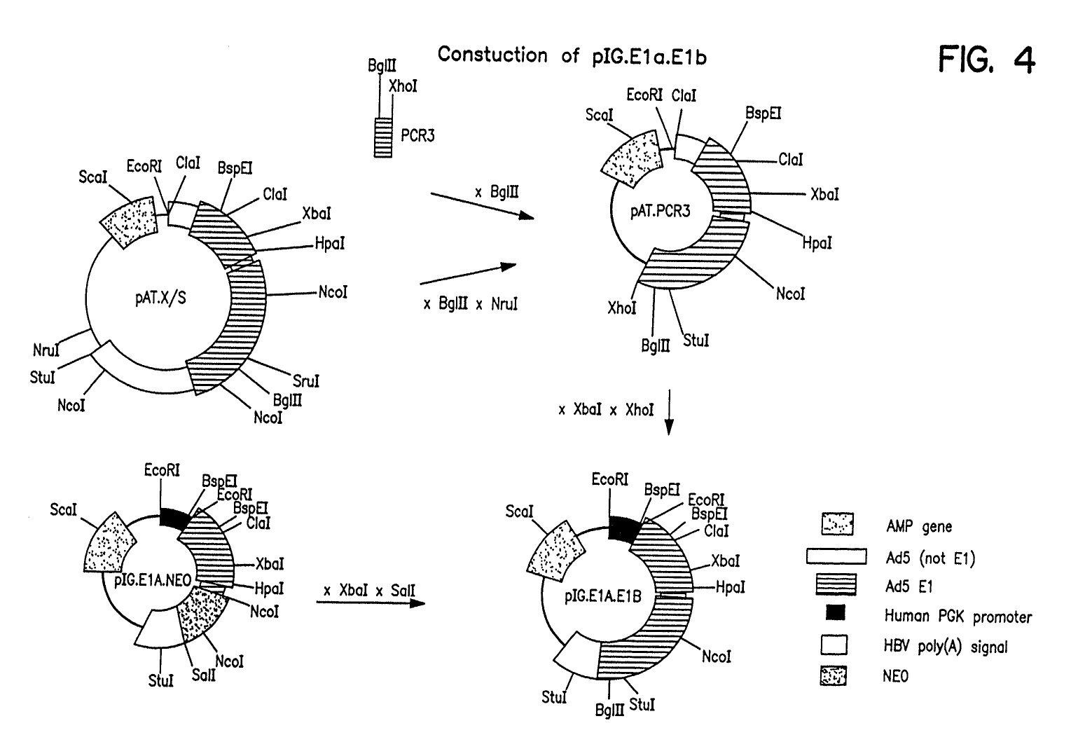

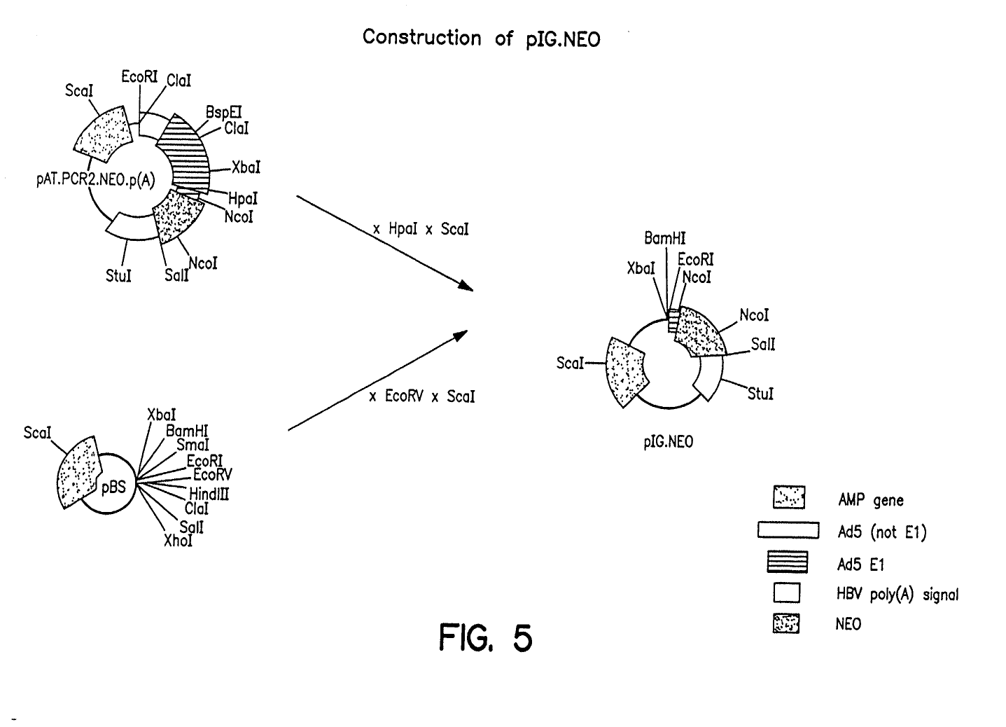

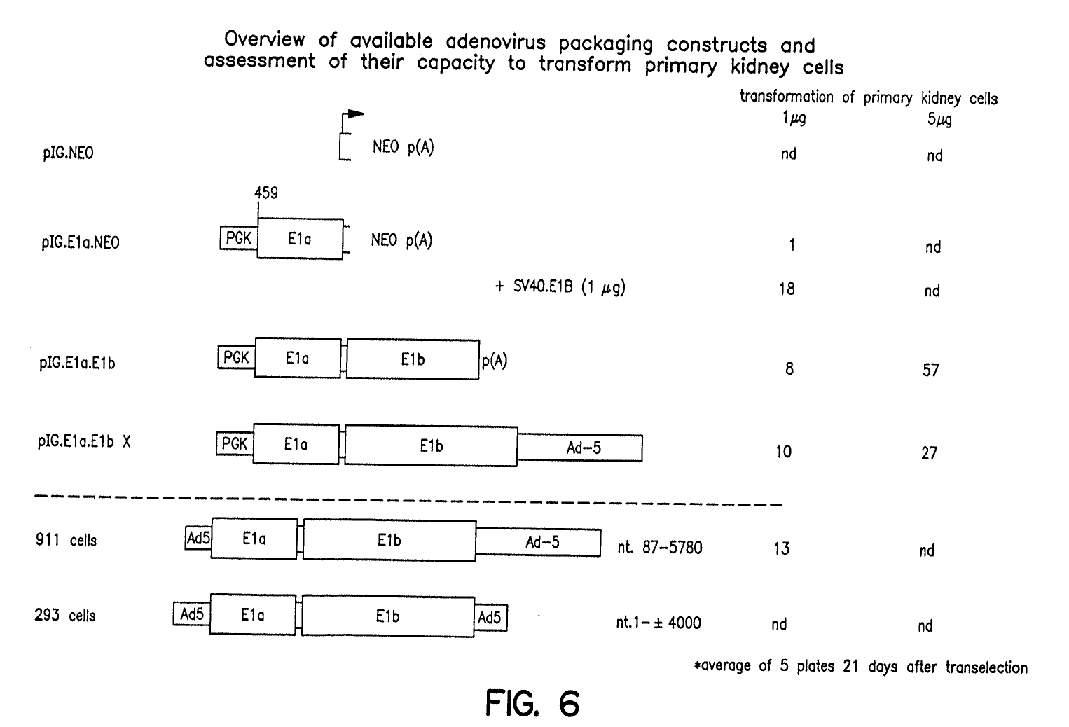

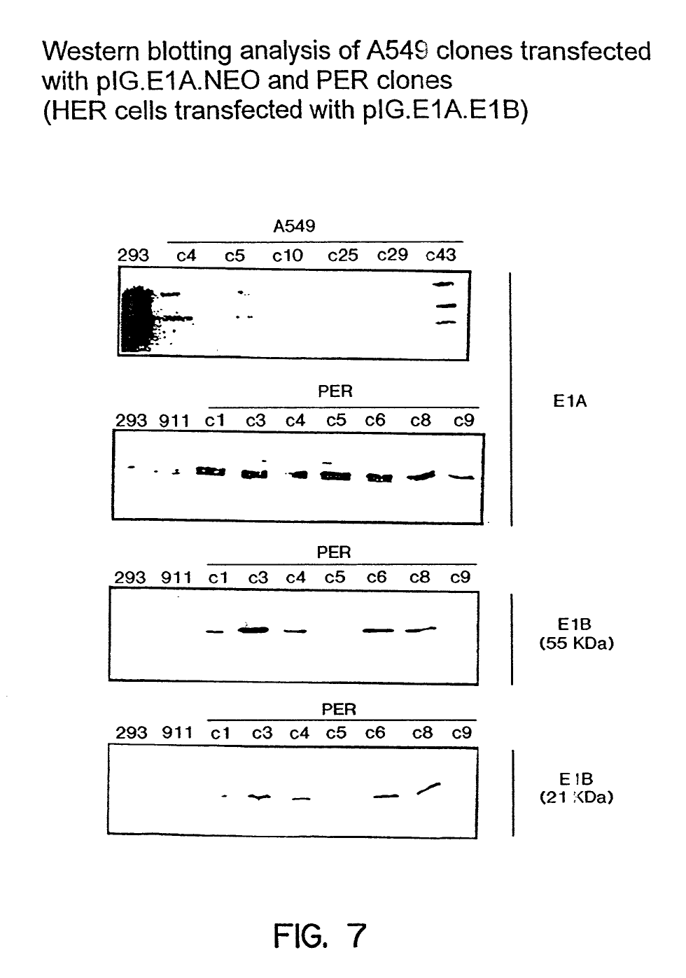

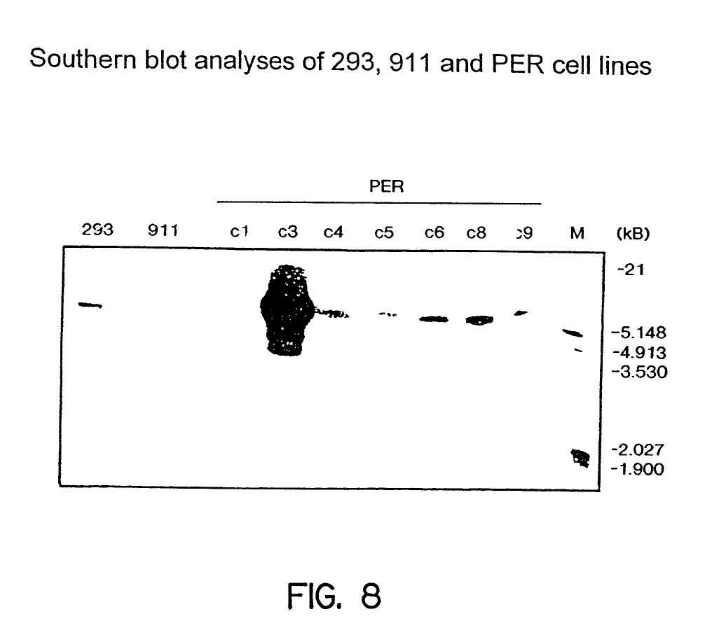

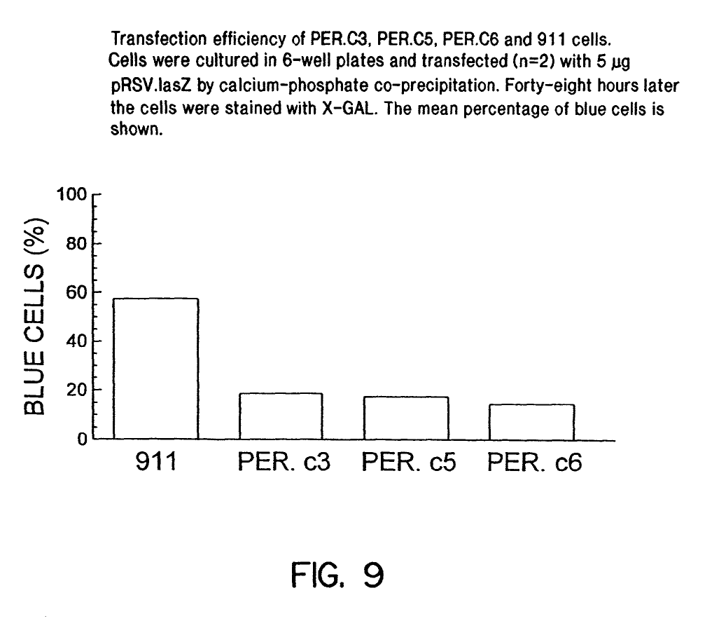

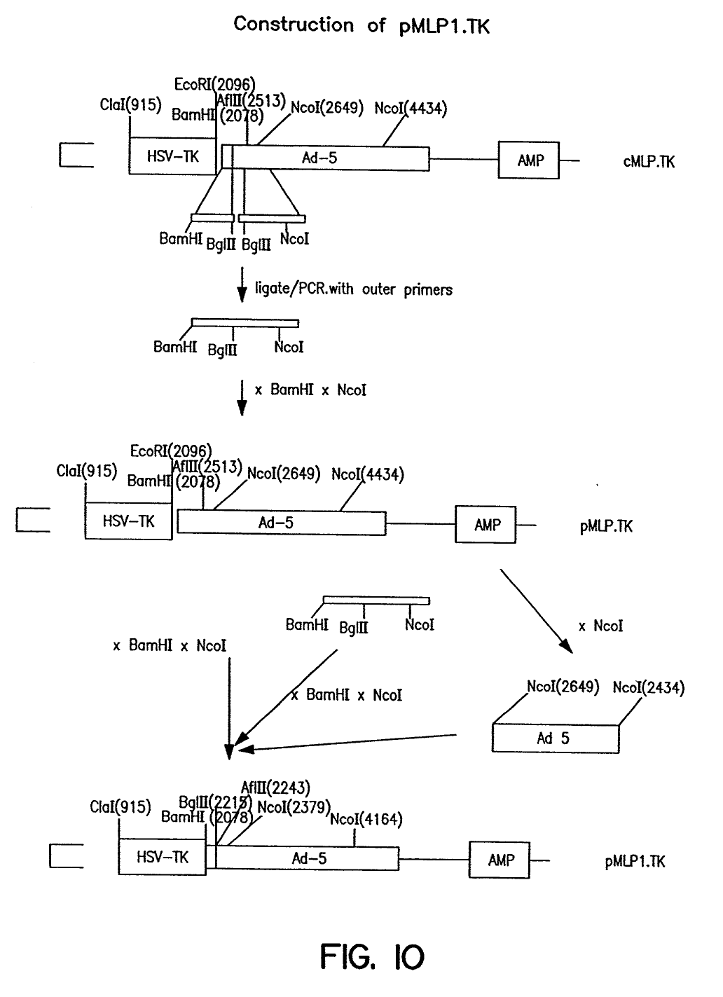

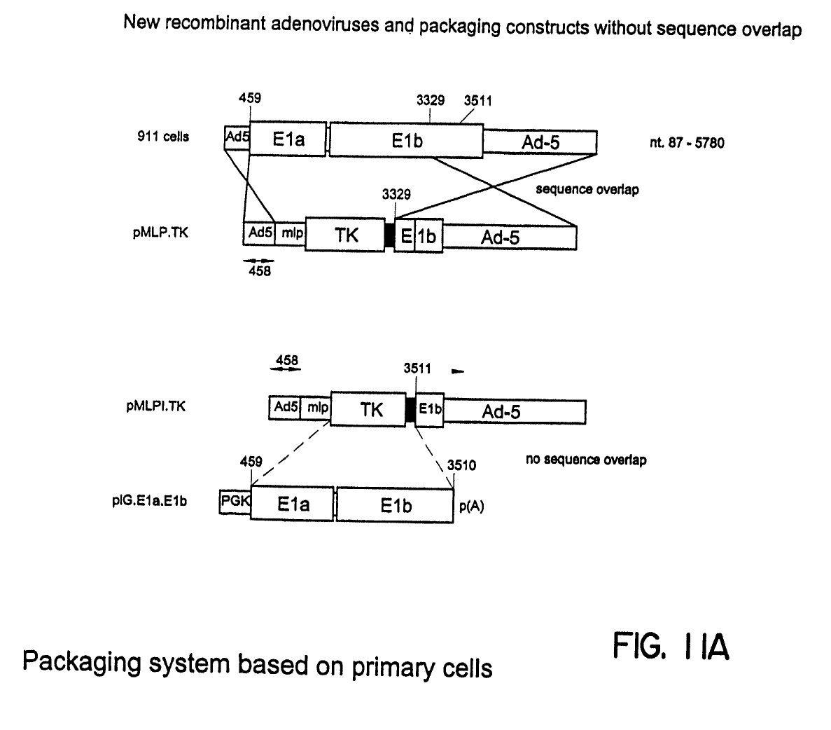

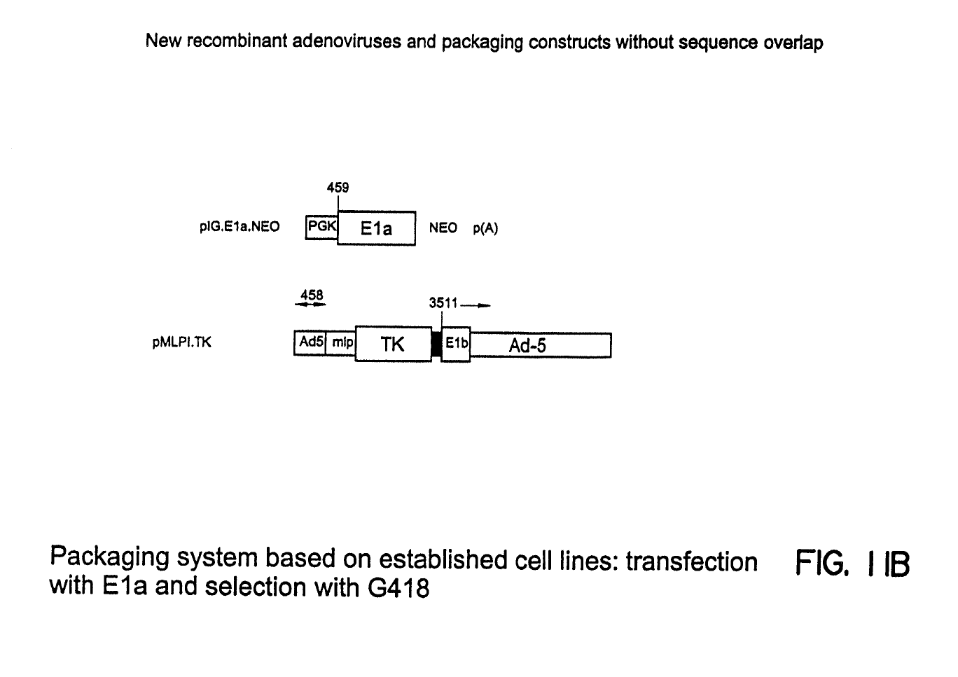

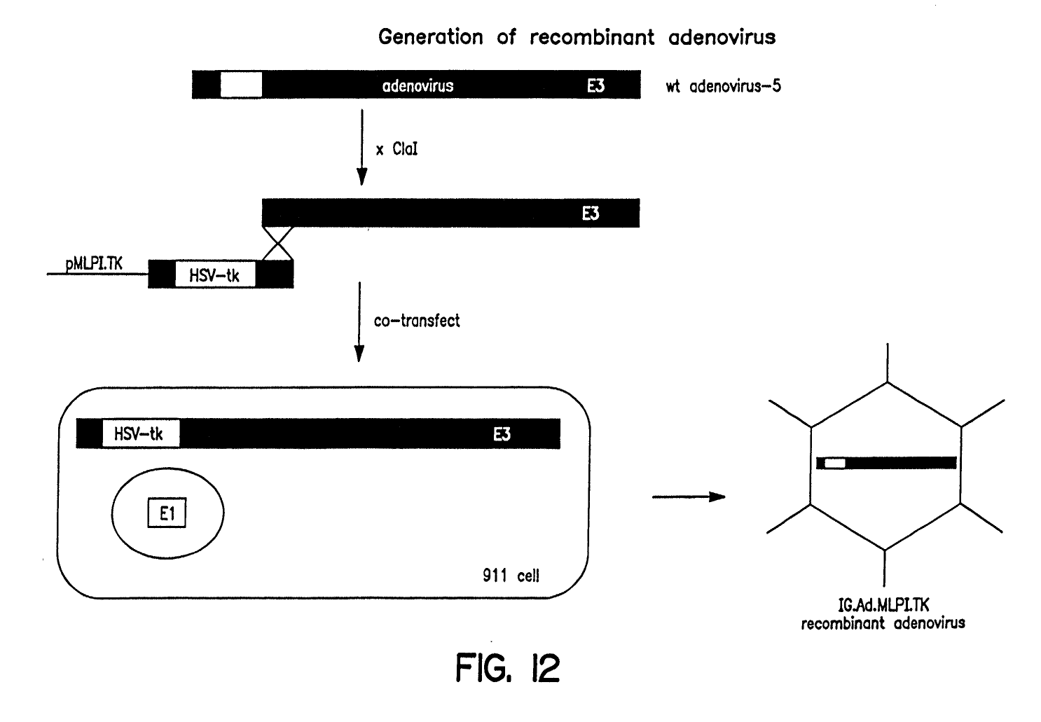

### DETAILED DESCRIPTION

The following definitions are used throughout the specification.

“Abnormal cell death” means an apoptosis-associated disorder which disorder is characterized by increased cell death due to malfunctioning of apoptotic cell death mechanisms.

Examples of abnormal cell death disorders or diseases that can be treated, prevented, and/or diagnosed by nucleic acids, polypeptides or antibodies of the present invention include, but are not limited to neuro-degenerative diseases such as Alzheimer's disease, Parkinson's disease, amyotrophic lateral sclerosis, retinitis pigmentosa, and cerebellar degeneration, myelodysplastic syndromes such as aplastic anemia, infectious or genetic immunodeficiencies such as acquired immunodeficiency syndrome, ischemic injuries such as myocardial infarction, stroke, and reperfusion injury, toxin-induced diseases such as alcohol-induced liver damage, cirrhosis, and lathyrism, wasting diseases such as cachexia, viral infections such as those caused by hepatitis B and C, and osteoporosis.

“Apoptosis” means cell death by means of the cell's regulatory mechanism, otherwise referred to as “programmed” cell death.

“Apoptosis-associated disorders” means any human or animal disease or disorder, affecting any one or any combination of organs, cavities, or body parts, which disorder is characterized by proliferative disorders or abnormal cell death related to the malfunctioning of apoptotic cellular regulation.

“Carrier” means a non-toxic material used in the formulation of pharmaceutical compositions to provide a medium, bulk and/or useable form to a pharmaceutical composition. A carrier may comprise one or more of such materials such as an excipient, stabilizer, or an aqueous pH buffered solution. Examples of physiologically acceptable carriers include aqueous or solid buffer ingredients including phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Compound” is used herein in the context of a “test compound” or a “drug candidate compound” described in connection with the screening assays of the present invention. As such, these compounds comprise organic or inorganic compounds, derived synthetically or from natural sources. The compounds include inorganic or organic compounds such as polynucleotides or hormone analogs that are characterized by relatively low molecular weights. Other biopolymeric organic test compounds include ribozymes, peptides comprising from about 2 to about 40 amino acids and larger polypeptides comprising from about 40 to about 500 amino acids, such as antibodies or antibody conjugates.

“Disease” means the overt presentation of symptoms (i.e., illness) or the manifestation of abnormal clinical indicators (e.g., biochemical indicators), resulting from defects in apoptosis-associated processes of E2F action. Alternatively, the term “disease” refers to a genetic or environmental risk of- or propensity for developing such symptoms or abnormal clinical indicators. Diseases associated with defects in E2F activation include, but are not limited to apoptosis-associated disorders, which include proliferative disorders and abnormal cell death diseases.

“Expressible nucleic acid” means a nucleic acid coding for a proteinaceous molecule, an RNA molecule, or a DNA molecule.

“Hybridization” refers to any process by which a strand of nucleic acid binds with a complementary strand through base pairing. The term “hybridization complex” refers to a complex formed between two nucleic acid sequences by virtue of the formation of hydrogen bonds between complementary bases. A hybridization complex may be formed in solution (e.g., Cot or Rot analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed). The term “stringent conditions” refers to conditions that permit hybridization between polynucleotides and the claimed polynucleotides. Stringent conditions can be defined by salt concentration, the concentration of organic solvent, e.g., formamide, temperature, and other conditions well known in the art. In particular, stringency can be increased by reducing the concentration of salt, increasing the concentration of formamide, or raising the hybridization temperature.

“Mammal” means any animal classified as a mammal, including humans, domestic and farm animals, and zoo, sports, or pet animals, such as dogs, horses, cats, hamsters, rats, mice, cattle pigs, goats, sheep, etc.

“Polynucleotide” means a polynucleic acid, in single or double stranded form, and in the sense or antisense orientation, complementary polynucleic acids that hybridize to a particular polynucleic acid under stringent conditions, and polynucleotides that are homologous in at least about 60 percent of its base pairs, and more preferably 70 percent of its base pairs are in common. The polynucleotides include polyribonucleic acids, polydeoxyribonucleic acids, and synthetic analogues thereof. The polynucleotides are described by sequences that vary in length, that range from about 10 to about 5000 bases, preferably about 100 to about 4000 bases, more preferably about 250 to about 2500 bases. A preferred polynucleotide embodiment comprises from about 10 to about 30 bases in length. A special embodiment of polynucleotide is the polyribonucleotide of from about 10 to about 22 nucleotides, more commonly described as small interfering RNAs (siRNAs).

“Proliferative disorders” means an apoptosis-associated disorder which disorder is characterized by single or multiple local abnormal or uncontrolled proliferation of cells, groups of cells, or tissues, whether benign or malignant, and which cells may also be described as “neoplastic”.

Examples of proliferative disorders or diseases that can be treated, prevented, and/or diagnosed by nucleic acids, polypeptides or antibodies of the present invention include, but are not limited to, various types of solid and liquid tumor growth, such as retinoblastoma, osteosarcoma, adenocarcinoma, leukemia, lymphoma, melanoma, myeloma, sarcoma, and teratocarcinoma, tumors of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus, hyperplasias of the thyroid, endometrium, pituitary gland and adrenal gland, lipodystrophia, lymphoproliferative diseases, psoriasis and vascular disorders such as atherosclerosis and restenosis, transplant-related myeloproliferative diseases, lymphocytosis and immunoproliferative diseases related to infection and autoimmune diseases, granulomatous diseases, like, for instance, histiocytosis and sarcoidosis, fibromatosis, multicentric histiocytosis, polycythaemia, and thrombocythaemia.

“Proliferative induction” means the induction of proliferation in cells (not characterized as “neoplastic”), groups of cells, or tissues, whether or not it occurs in vivo or ex vivo. Examples of diseases that can be treated, prevented or diagnosed by nucleic acids, polypeptides or antibodies of the present invention include, but are not limited to, anemia, lymphocytopenia, thrombopenia, and neutropenia. Also several treatments, like stem cell therapy, transplantation (e.g., of Langerhans cells), tissue repair (e.g., bone repair and bone replacement), and corrective surgery, might greatly benefit from an induction of proliferation in cells, groups of cells, or tissues. Similarly, induction of proliferation in cardiac myocytes can also be beneficial to prevent or treat hypertrophy.

“Treatment” means an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. Administration “in combination with” or “admixture with” one or more further therapeutic agents includes simultaneous (concurrent) and consecutive administration in any order.

Library Screening for E2F-Related Functional Genes

The present invention, in one embodiment, provides methods that use a library of expressible nucleic acids comprising a multiplicity of compartments. Each compartment comprises at least one vehicle including at least one nucleic acid of the library, whereby the vehicle is capable of introducing at least one nucleic acid into a cell such that it can be expressed. Another advantage of the library is that it includes a multiplicity of compartments each including at least one nucleic acid. When a compartment includes only one nucleic acid, then it is known that the unique nucleic acid in the distinct compartment is responsible for whatever change in phenotype is observed.

In one embodiment, at least one compartment includes at least two vehicles. Especially with, but not limited to, large libraries, it becomes advantageous to reduce the number of compartments to reduce the number of screening assays that need to be performed. In such cases, libraries are provided that include more than one vehicle. If after screening, a certain effect is correlated to a certain compartment, the vehicles in the compartment may be analysed separately in an additional screening assay to select the vehicle including the nucleic acid the expression of which exerts the effect. In addition, the presence of more than one vehicle in a compartment may be advantageous when a library containing one vehicle per compartment is screened for a nucleic acid capable of exerting an effect in combination with one particular other nucleic acid. The other nucleic acid may then be provided to the cell by adding a vehicle including the particular other nucleic acid to all compartments prior to performing the screening assay. Similarly, the vehicle may include at least two nucleic acids.

The library used in the method may use any kind of cell. Preferably, when the library is screened for the presence of nucleic acids with potential therapeutic values, the cell is a eukaryotic cell, especially a mammalian cell. Examples of suitable cells include hepatomas: HepG2; keratinocytes: HCAT1; osteosarcomas: U2OS, SaOS; cervixcarcinoma: Hela; breast tumor: MCF7, T47D, MDA-MB-468; pancreatic tumor: BxPC3, HPAC; colon carcinoma: COLO205, HT29; melanomas: SK-MEL-2, M14; leukemia cells: K562, TF1, Daubi, Raji; central nervous system: SF-268; lung tumor: A549, SW1573; prostate: PC-3, DU-145; bladder: HT-1376; stomach: Hs740.T; and kidney: Caki-1. In a preferred embodiment, the cells are divided over a number of compartments each including at least one vehicle including at least one nucleic acid from the library. The number of compartments preferably corresponds to the multiplicity of compartments in the library.

In a preferred embodiment, the vehicle includes a viral element or a functional part, derivative and/or analogue thereof. A viral element may include a virus particle such as, but not limited to, an enveloped retrovirus particle or a virus capsid of a non-enveloped virus such as, but not limited to, an adenovirus. A virus particle is favorable since it allows the efficient introduction of at least one nucleic acid into a cell. A viral element may also include a viral nucleic acid allowing the amplification of the library in cells. A viral element may include a viral nucleic acid allowing the packaging of at least one nucleic acid into a vehicle, where the vehicle is a virus particle. In a preferred embodiment, the viral element is derived from an adenovirus. Preferably, the vehicle includes an adenoviral vector packaged into an adenoviral capsid, or a functional part, derivative, and/or analogue thereof. Adenovirus biology is also comparatively well known on the molecular level. Many tools for adenoviral vectors have been and continue to be developed, thus making an adenoviral capsid a preferred vehicle for incorporating in a library of the invention. An adenovirus is capable of infecting a wide variety of cells. However, different adenoviral serotypes have different preferences for cells. To combine and widen the target cell population that an adenoviral capsid of the invention can enter in a preferred embodiment, the vehicle includes adenoviral fiber proteins from at least two adenoviruses.

In a preferred embodiment, the nucleic acid derived from an adenovirus includes the nucleic acid encoding an adenoviral late protein or a functional part, derivative, and/or analogue thereof. An adenoviral late protein, for instance an adenoviral fiber protein, may be favorably used to target the vehicle to a certain cell or to induce enhanced delivery of the vehicle to the cell. Preferably, the nucleic acid derived from an adenovirus encodes for essentially all adenoviral late proteins, enabling the formation of entire adenoviral capsids or functional parts, analogues, and/or derivatives thereof. Preferably, the nucleic acid derived from an adenovirus includes the nucleic acid encoding adenovirus E2A or a functional part, derivative, and/or analogue thereof. Preferably, the nucleic acid derived from an adenovirus includes the nucleic acid encoding at least one E4-region protein or a functional part, derivative, and/or analogue thereof, which facilitates, at least in part, replication of an adenoviral derived nucleic acid in a cell.

In one embodiment, the nucleic acid derived from an adenovirus includes the nucleic acid encoding at least one E1-region protein or a functional part, derivative, and/or analogue thereof. The presence of the adenoviral nucleic acid encoding an E1-region protein facilitates, at least in part, replication of the nucleic acid in a cell. The replication capacity is favored in certain applications when screening is done for expressible nucleic acids capable of irradiating tumor cells. In such cases, replication of an adenoviral nucleic acid leading to the amplification of the vehicle in a mammal including tumor cells may lead to the irradiation of metastasized tumor cells. On the other hand, the presence of an adenoviral nucleic acid encoding an E1-region protein may facilitate, at least in part, amplification of the nucleic acid in a cell for the amplification of vehicles including the adenoviral nucleic acid. In one embodiment, the vehicle further includes a nucleic acid including an adeno-associated virus terminal repeat or a functional part, derivative, and/or analogue thereof which allows the integration of at least one nucleic acid in a cell.

The present invention provides a method for identifying apoptosis-associated functions of the unique nucleic acids present in a library, the functions of which are for the most part unknown, or at least not completely understood. This method transduces multiple subpopulations of cells, each subpopulation present in a discrete compartment of the library, with at least one vehicle including at least one nucleic acid from the library, culturing the cells while allowing for expression of the nucleic acid, and determining the expressed function. The library is screened for the presence of expressible nucleic acids capable of influencing, at least in part, the activity of E2F.

The present method preferably utilizes a set of adapter plasmids by inserting a set of cDNAs, DNAs, ESTs, genes, synthetic oligonucleotides, or a library of nucleic acids into E1-deleted adapter plasmids; cotransfecting an E1-complementing cell line with the set or library of adapter plasmids and at least one plasmid having sequences homologous to sequences in the set of adapter plasmids and which also includes all adenoviral genes not provided by the complementing cell line or adapter plasmids necessary for replication and packaging to produce a set or library of recombinant adenoviral vectors preferably in a miniaturized, high throughput setting. The plasmid-based system is used to rapidly produce adenoviral vector libraries that are preferably replication competent adenovirus (“RCA”)-free for high throughput screening. Each step of the method can be performed in a multiwell format and automated to further increase the capacity of the system. This high throughput system facilitates expression analysis of a large number of sample nucleic acids from human and other organisms both in vitro and in vivo and is a significant improvement over other available techniques in the field.

The method permits the amplification of the vehicles including the unique nucleic acids present in a library. Such amplification may be achieved culturing the cell with the vehicle, allowing the amplification of the vehicle, and harvesting vehicles amplified by the cell. Preferably, the cell is a primate cell thereby enabling the amplification of vehicles including viral elements that allow replication of the vehicle nucleic acid. Preferably, the cell includes a nucleic acid encoding an adenoviral E1-region protein thereby allowing, among other things, the amplification of vehicles including viral elements derived from adenovirus including adenoviral nucleic acids including a functional deletion of at least part of the E1-region. Preferably, the cell is a PER.C6 cell (ECACC deposit number 96022940) or a functional derivative and/or analogue thereof. A PER. C6 cell (or a functional derivative and/or analogue thereof) allows the replication of adenoviral nucleic acid with a deletion of the E1-coding region without concomitant production of RCA in instances wherein the adenoviral nucleic acid and chromosomal nucleic acid in the PER.C6 cell or functional derivative and/or analogue thereof do not include sequence overlap that allows for homologous recombination between the adenoviral and chromosomal nucleic acid leading to the formation of RCA. Preferably, the cell further includes nucleic acid encoding adenovirus E2A and/or an adenoviral E4-region protein or a functional part, derivative, and/or analogue thereof. This allows the replication of adenoviral nucleic acid with functional deletions of nucleic acid encoding adenovirus E2A and/or an adenoviral E4-region protein, thereby inhibiting replication of the adenoviral nucleic acid in a cell not including nucleic acid encoding adenovirus E2A and/or an adenoviral E4-region protein or a functional part, derivative and/or analogue thereof, for instance a cell capable of displaying a certain function.

In a preferred method, the vehicle nucleic acid does not include sequence overlap with other nucleic acids present in the cell, leading to the formation of vehicle nucleic acid capable of replicating in the absence of E1-region encoded proteins.

The method is preferably implemented using a multiplicity of compartments in a multiwell format. A multiwell format is very suited for automated execution of at least part of the methods of the invention.

The present invention uses high throughput generation of recombinant adenoviral vector libraries containing one or more sample nucleic acids, followed by high throughput screening of the adenoviral vector libraries in a host to alter the phenotype of the host as a means of assigning a function to expression product(s) of the sample nucleic acids. Libraries of E1-deleted adenoviruses are generated in a high throughput setting using nucleic acid constructs and transcomplementary packaging cells. The sample nucleic acid libraries can be a set of distinct defined or undefined sequences or can be a pool of undefined or defined sequences. The first nucleic acid construct is a relatively small and easy to manipulate adapter plasmid containing, in an operable configuration, at least a left ITR, a packaging signal, and an expression cassette with the sample nucleic acids. The second nucleic acid construct contains one or more nucleic acid molecules that partially overlap with each other and/or with sequences in the first construct. The second construct also contains at least all adenovirus sequences necessary for replication and packaging of a recombinant adenovirus not provided by the adapter plasmid or packaging cells. The second nucleic acid construct is deleted in E1-region sequences and optionally E2B region sequences other than those required for virus generation and/or E2A, E3 and/or E4 region sequences. Cotransfection of the first and second nucleic acid constructs into the packaging cells leads to homologous recombination between overlapping sequences in the first and second nucleic acid constructs and among the second nucleic acid constructs when it is made up of more than one nucleic acid molecule. Generally, the overlapping sequences are no more than 5000 bp and encompass E2B region sequences essential for virus production. Recombinant viral DNA is generated with an E1-deletion that is able to replicate and propagate in the E1-complementing packaging cells to produce a recombinant adenoviral vector library. The adenoviral vector library is introduced in a high throughput setting into a host which is grown to allow sufficient expression of the product(s) encoded by the sample nucleic acids to permit detection and analysis of its biological activity. The host can be cultured cells in vitro or an animal or plant model. Sufficient expression of the product(s) encoded by the sample nucleic acids alters the phenotype of the host. Using any of a variety of in vitro and/or in vivo assays for biological activity, the altered phenotype is analyzed and identified and a function is thereby assigned to the product(s) of the sample nucleic acids. The plasmid-based adenoviral vector systems described here provide for the creation of large gene-transfer libraries that can be used to screen for novel genes applicable to human diseases, such as those discussed in more detail herein. Identification of a useful or beneficial biological effect of a particular adenoviral mediated transduction can result in a useful gene therapeutic product or a target for a small molecule drug for treatment of such human diseases.

There are several advantages to the library used in the present invention over currently available techniques. The entire process lends itself to automation especially when implemented in a 96-well or other multi-well format. The high throughput screening, using a number of different in vitro assays, provides a means of efficiently obtaining functional information in a relatively short period of time. The member(s) of the recombinant adenoviral libraries that exhibit or induce a desired phenotype in a host in vitro or in situ are identified to reduce the libraries to a manageable number of recombinant adenoviral vectors or clones that can be tested in vitro in an animal model.

Another distinct advantage of the present library is that the adenoviral libraries produced are capable of being RCA-free. RCA contamination throughout the libraries could become a major obstacle, especially if libraries are continuously amplified for use in multiple screening programs. A further advantage of the subject invention is minimization of the number of steps involved in the process. The methods of the subject invention do not require cloning of each individual adenovirus before use in a high throughput-screening program. Other systems include one or more steps in E. coli to achieve homologous recombination for the various adenoviral plasmids necessary for vector generation (Chartier, et al (1996) J. Virol. 70(7):4805-10; Crouzet, et al. (1997) Proc. Natl. Acad. Sci. USA 94(4):1414-9; He, et al. (1998) Proc. Natl. Acad. Sci. USA 95(5):2509-14). Another plasmid system that has been used for adenoviral recombination and adenoviral vector generation, and which is based on homologous recombination in human cells, is the pBHG series of plasmids. However, if this plasmid is used in 293 cells, the plasmid can become unstable because the plasmid pBHG contains two ITRs close together and also can overlap with E1 sequences. All these features are undesirable and lead to RCA production or otherwise erroneous adenoviral vector production (Bett, et al. (1994) Proc. Natl. Acad. Sci. USA 91(19):8802-6). The recombinant nucleic acids of the subject invention have been designed to avoid constructions with these undesirable features.

A further advantage of the adenoviral library is the ability of recombinant adenoviruses to efficiently transfer and express recombinant genes in a variety of mammalian cells and tissues in vitro and in vivo, resulting in the high expression of the transferred sample nucleic acids. The ability to productively infect quiescent cells, further expands the utility of the recombinant adenoviral libraries. In addition, high expression levels ensure that the product(s) of the sample nucleic acids will be expressed to sufficient levels to induce a change that can be detected in the phenotype of a host and allow the function of the product(s) encoded by the sample nucleic to be determined.

The sample nucleic acids can be genomic DNA, cDNA, previously cloned DNA, genes, ESTs, synthetic double stranded oligonucleotides, or randomized sequences derived from one or multiple related or unrelated sequences. The sample nucleic acids can also be directly expressed as polypeptides, antisense nucleic acids, or genetic suppressor elements (GSE). The sample nucleic acid sequences can be obtained from any organism including mammals (for example, man, monkey, mouse), fish (for example, zebrafish, pufferfish, salmon), nematodes (for example, C. elegans), insects (for example, Drosophila), yeasts, fungi, bacteria, and plants. Alternatively, the sample nucleic acids are prepared as synthetic oligonucleotides using commercially available DNA synthesizers and kits. The strand coding the open reading frame of the polypeptide or product of the sample nucleic acid and the complementary strand are prepared individually and annealed to form double-stranded DNA. Special annealing conditions are not required. The sequences of the sample nucleic acids can be randomized or not through mutagenizing or methodologies promoting recombination. The sample nucleic acids code for a product(s) for which a biological activity is unknown. The phrase biological activity is intended to mean an activity that is detectable or measurable either in situ, in vivo, or in vitro. Examples of a biological activity include but are not limited to altered viability, morphologic changes, apoptosis induction, DNA synthesis, tumorigenesis, disease or drug susceptibility, chemical responsiveness or secretion, and protein expression.

The sample nucleic acids preferably contain compatible ends to facilitate ligation to the vector in the correct orientation and to operatively link the sample nucleic acids to a promoter. For synthetic double-stranded oligonucleotide ligation, the ends compatible to the vector can be designed into the oligonucleotides. When the sample nucleic acid is an EST, genomic DNA, cDNA, gene, or previously cloned DNA, the compatible ends can be formed by restriction enzyme digestion or the ligation of linkers to the DNA containing the appropriate restriction enzyme sites. Alternatively, the vector can be modified by the use of linkers. The restriction enzyme sites are chosen so that transcription of the sample nucleic acid from the vector produces the desired product, i.e., polypeptide, antisense nucleic acid, or GSE.

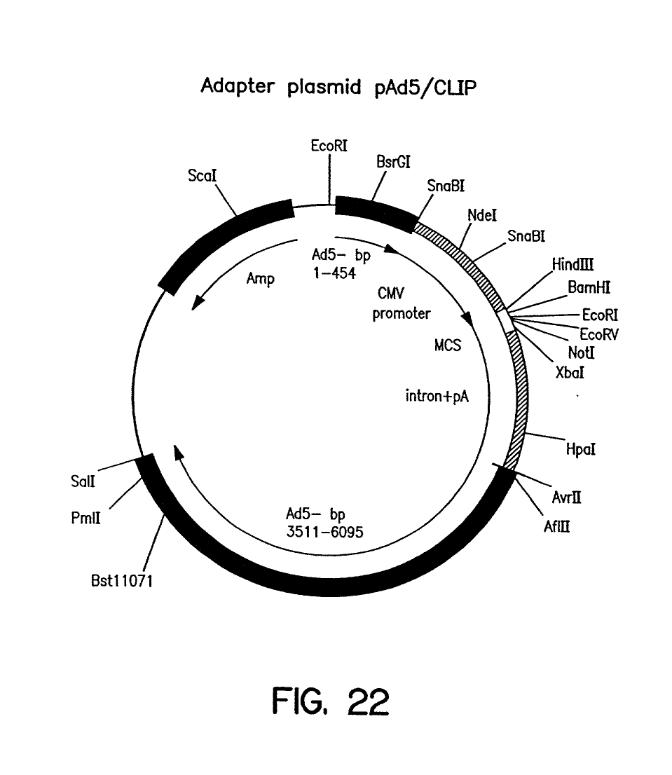

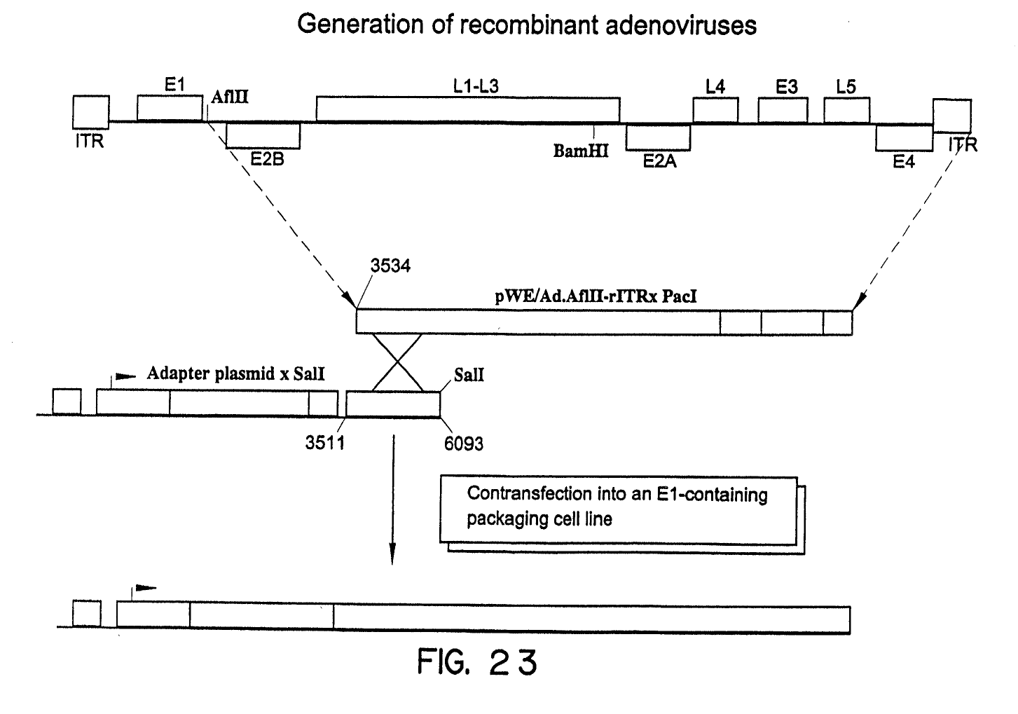

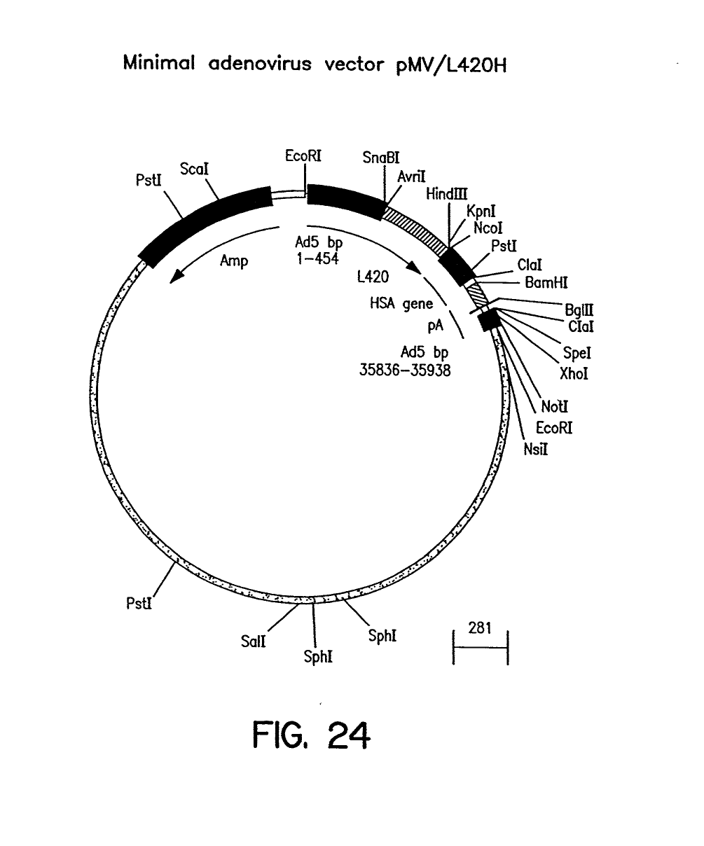

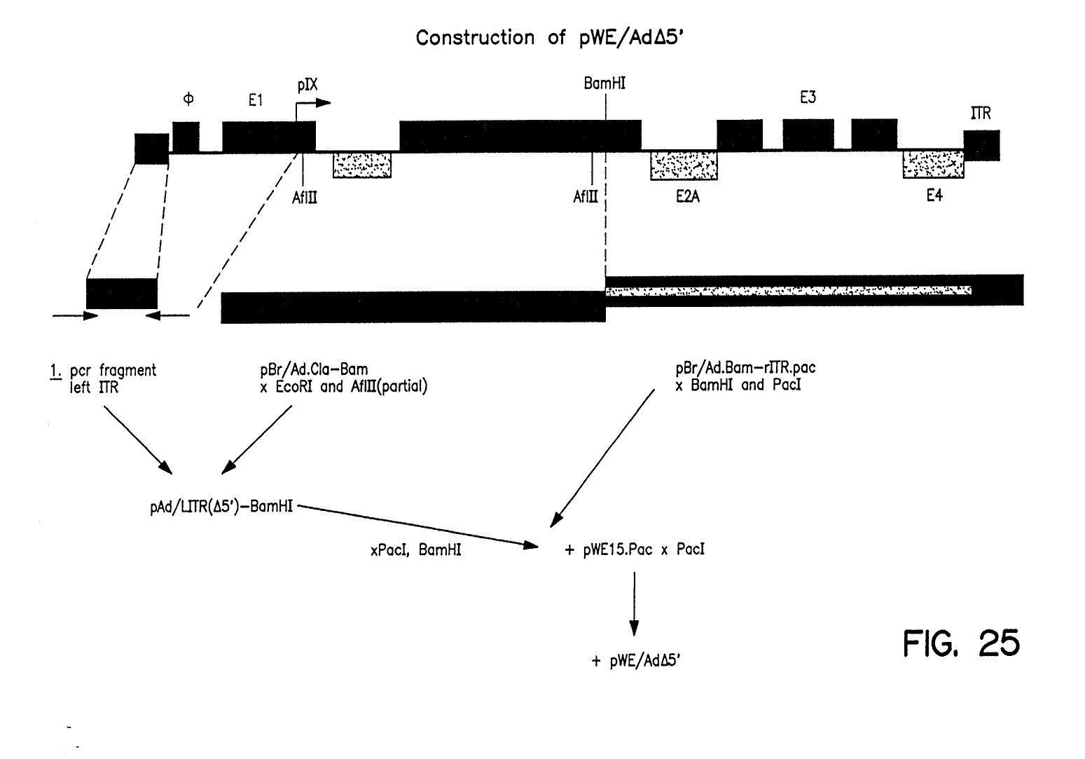

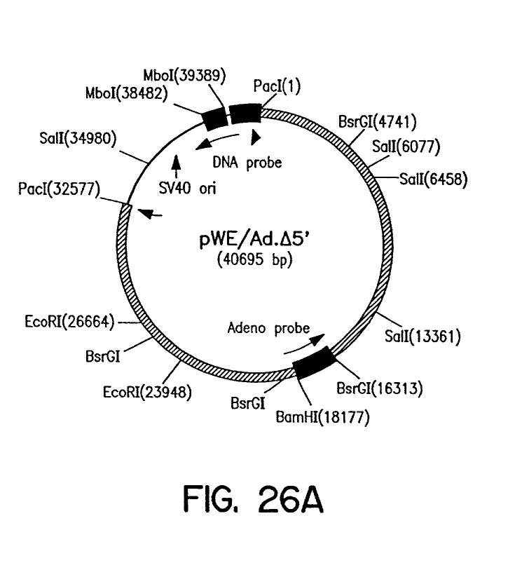

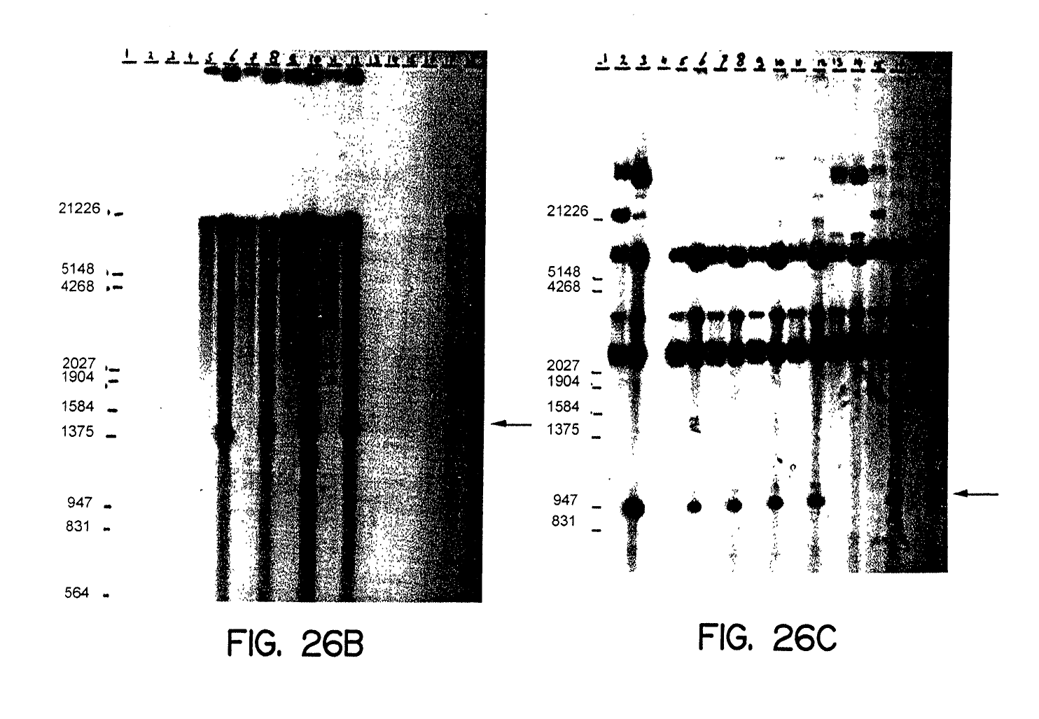

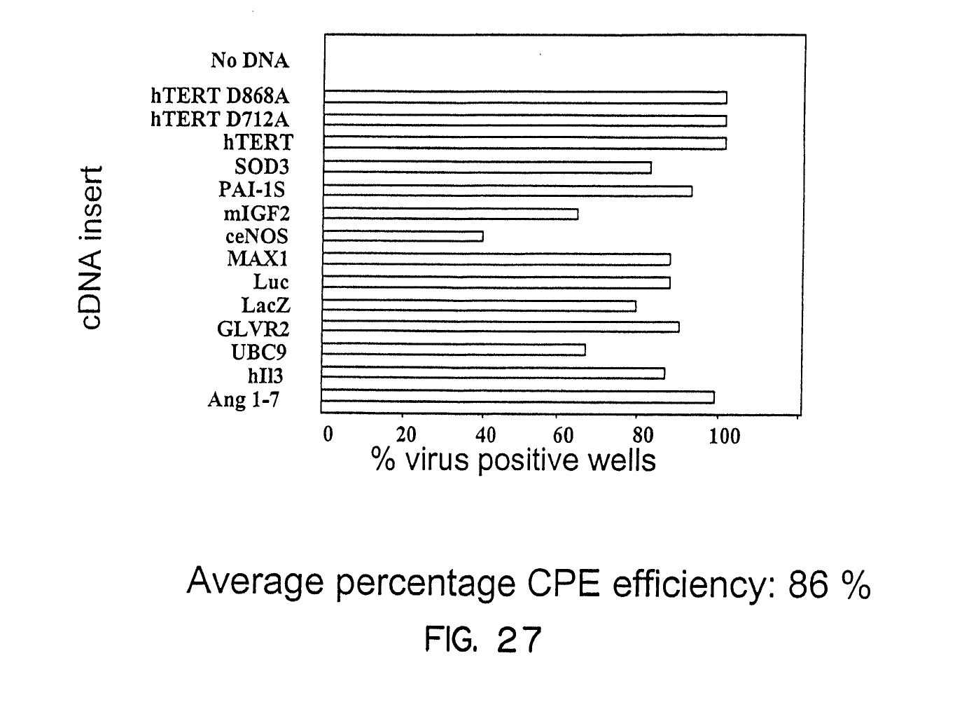

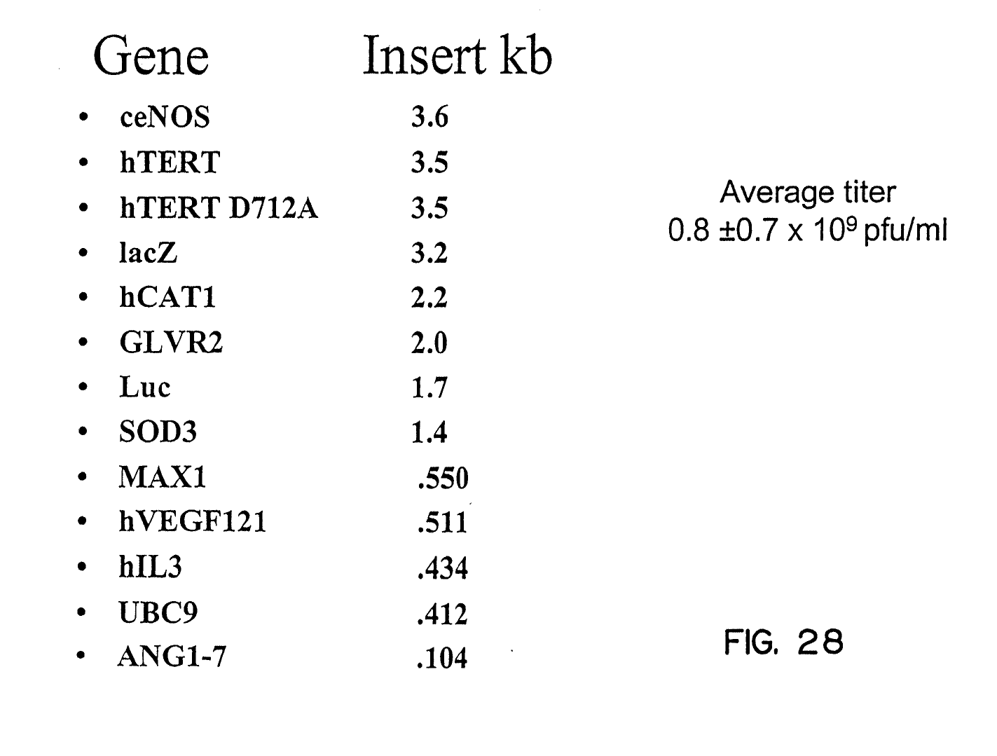

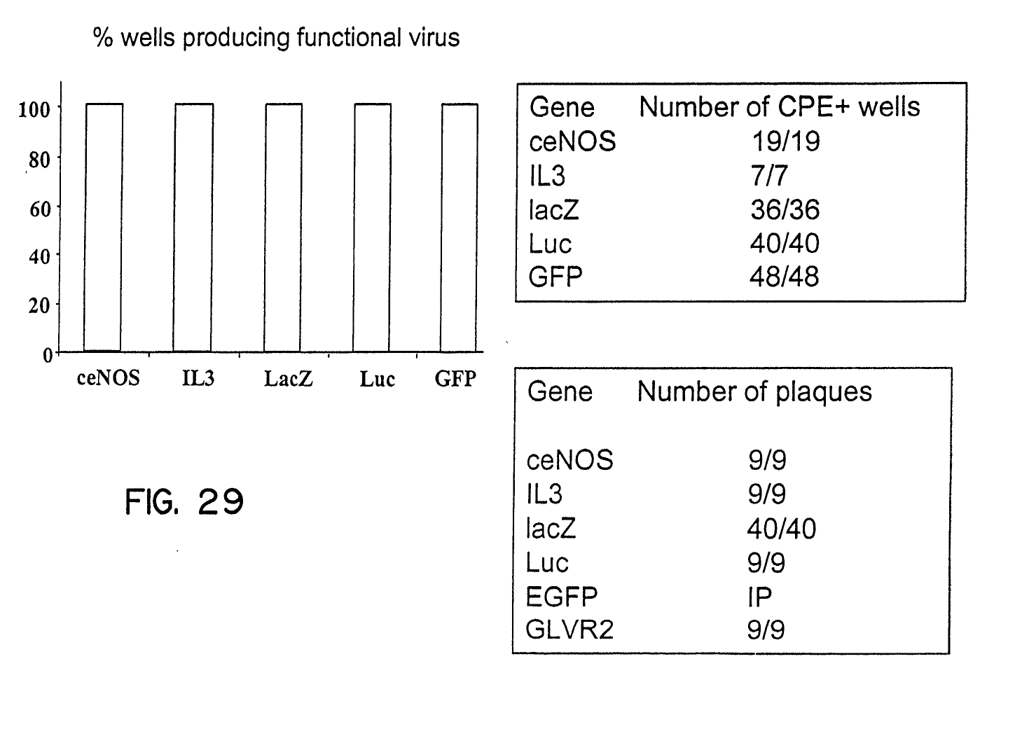

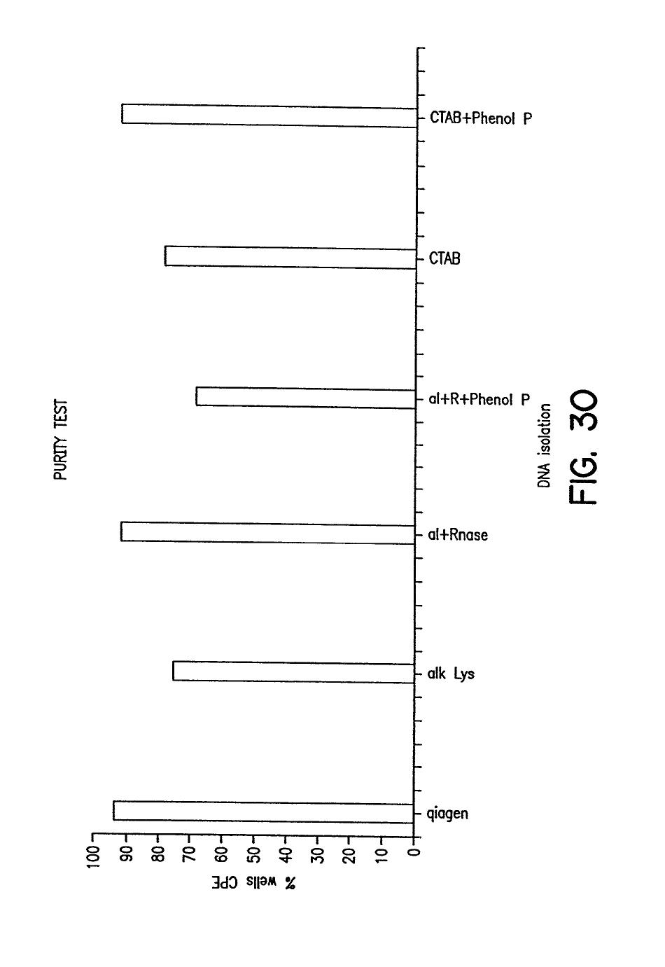

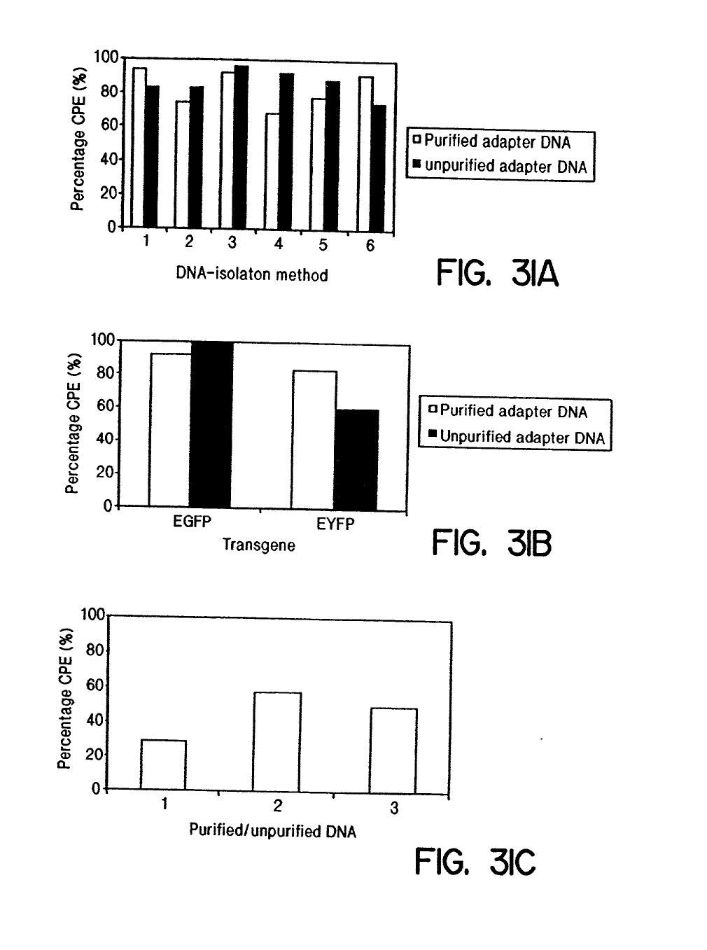

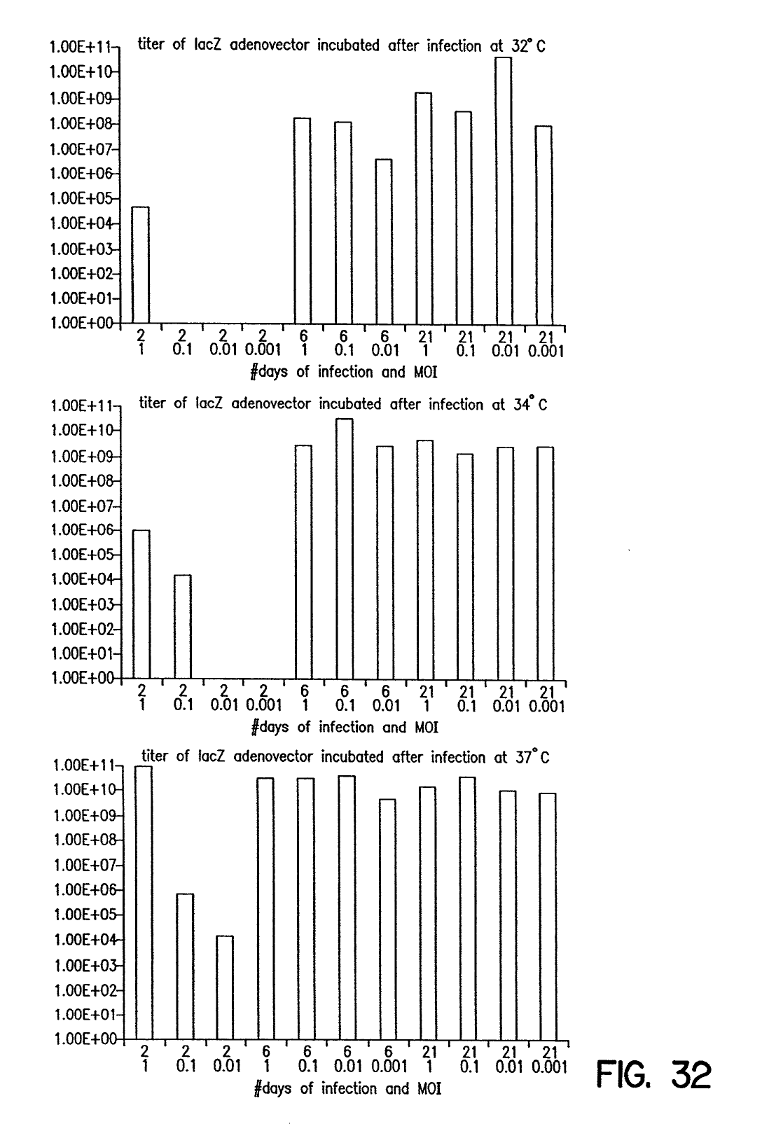

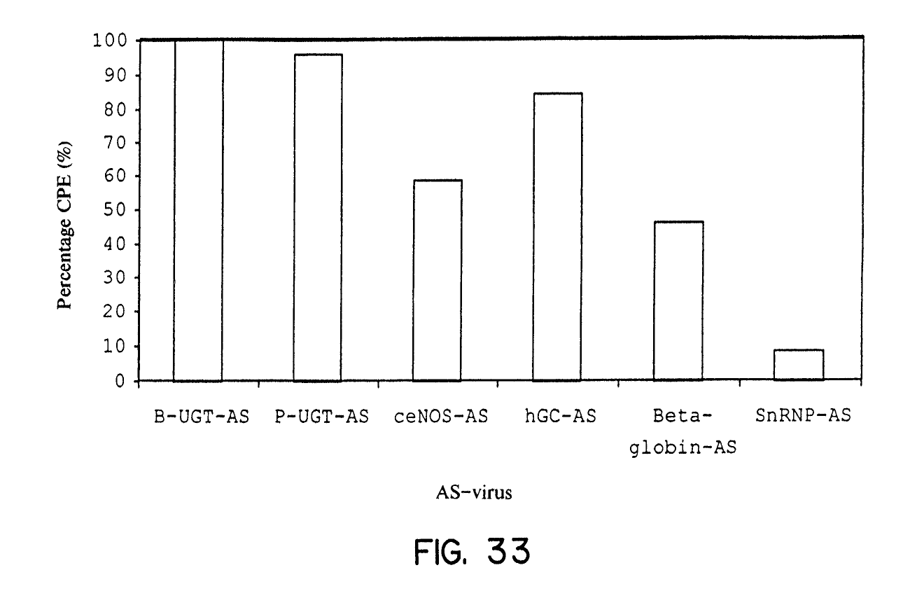

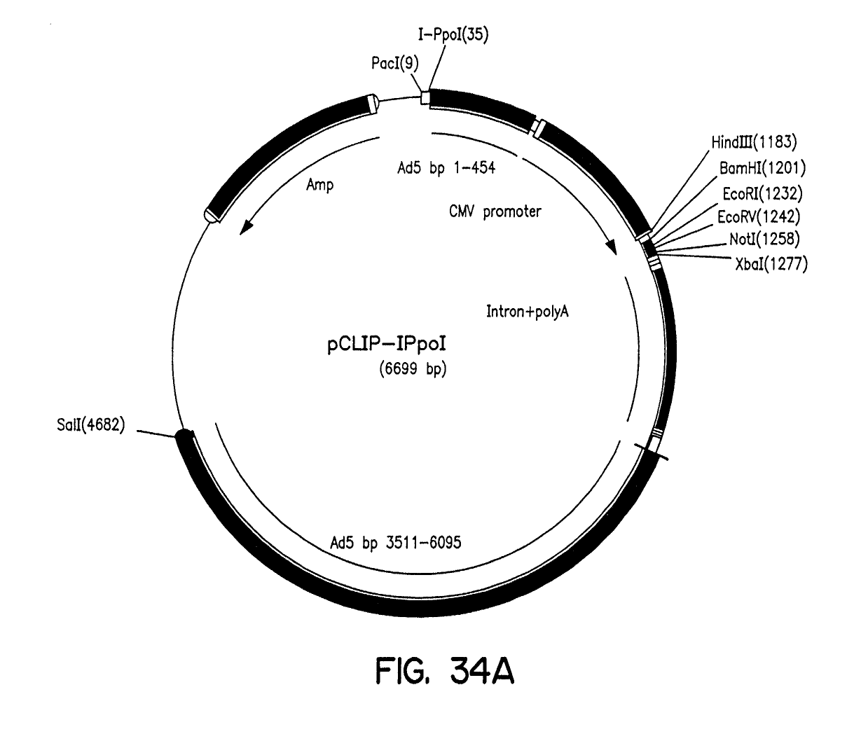

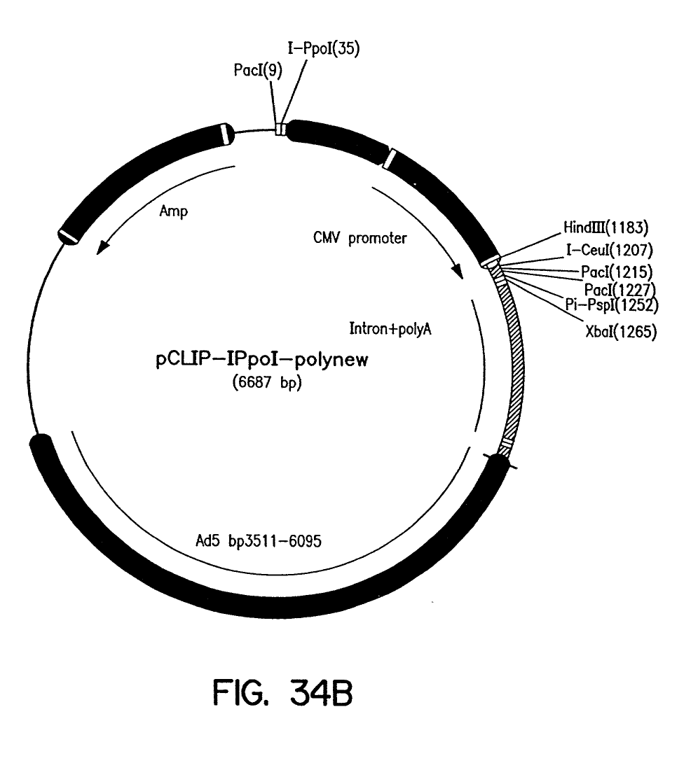

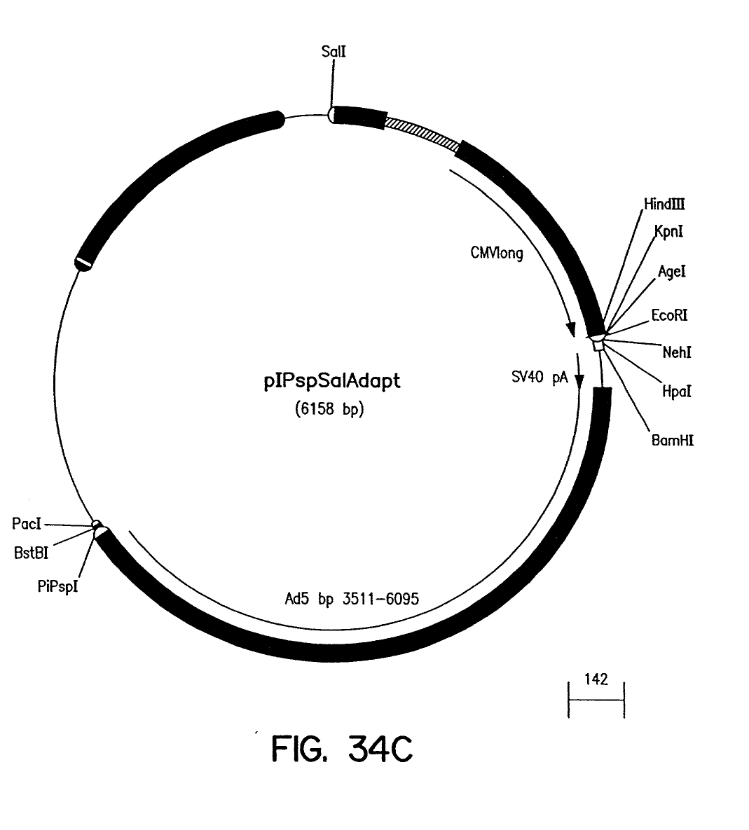

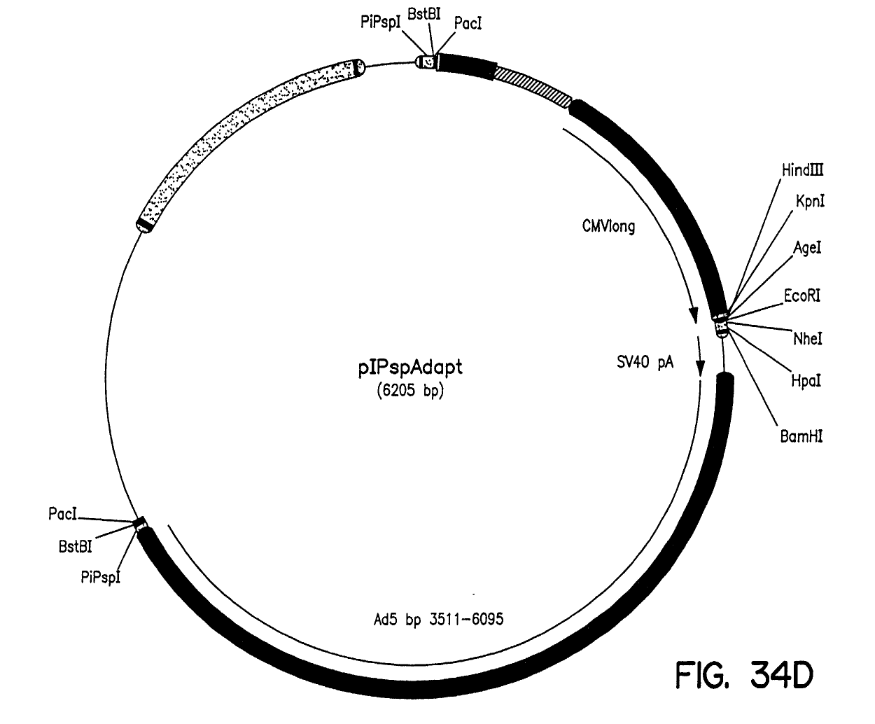

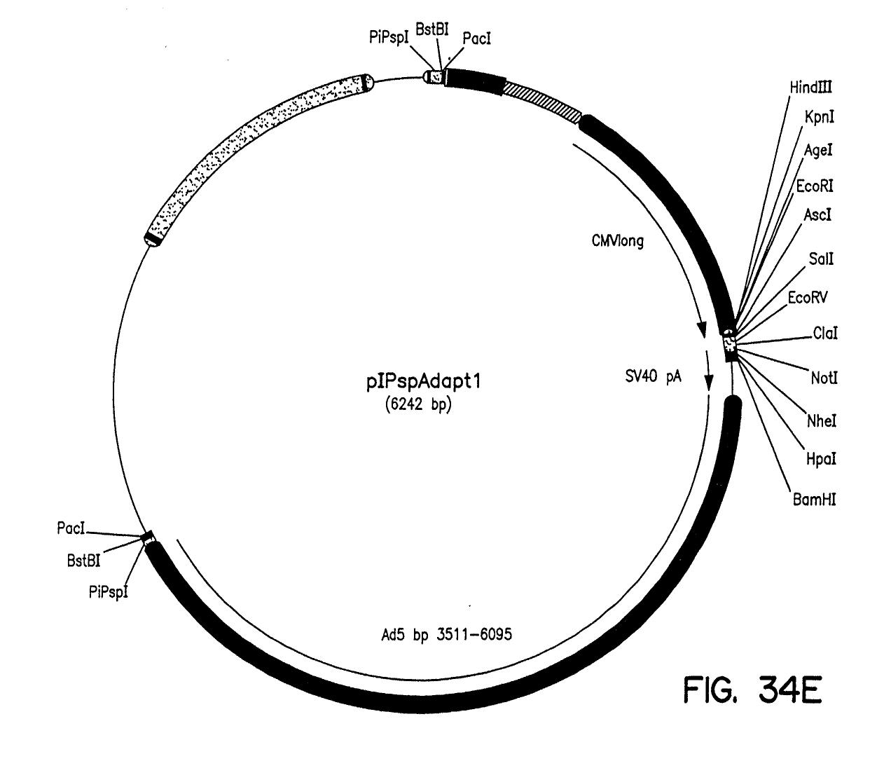

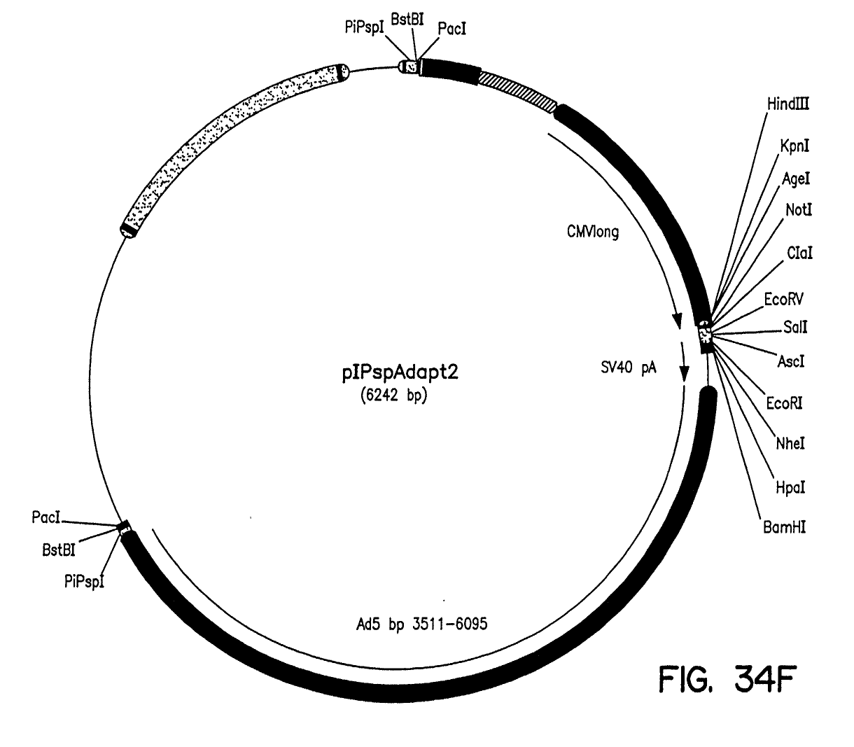

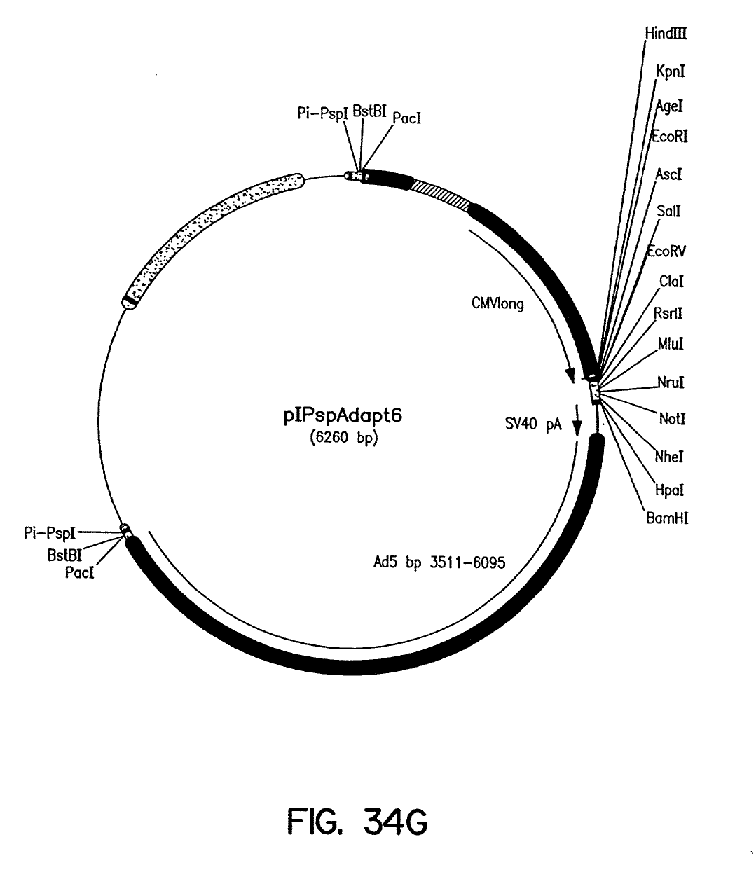

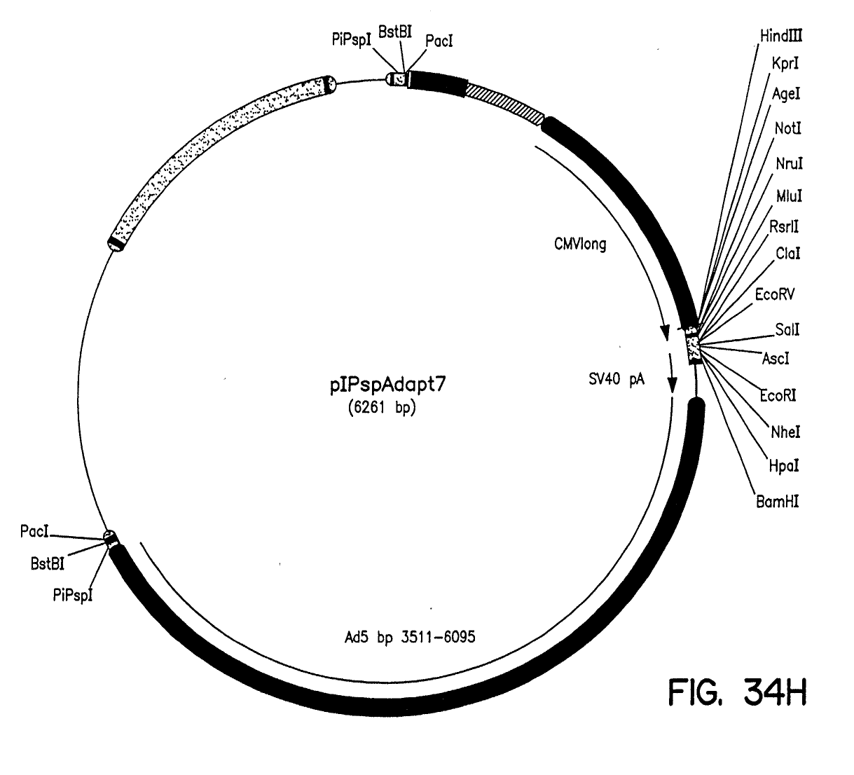

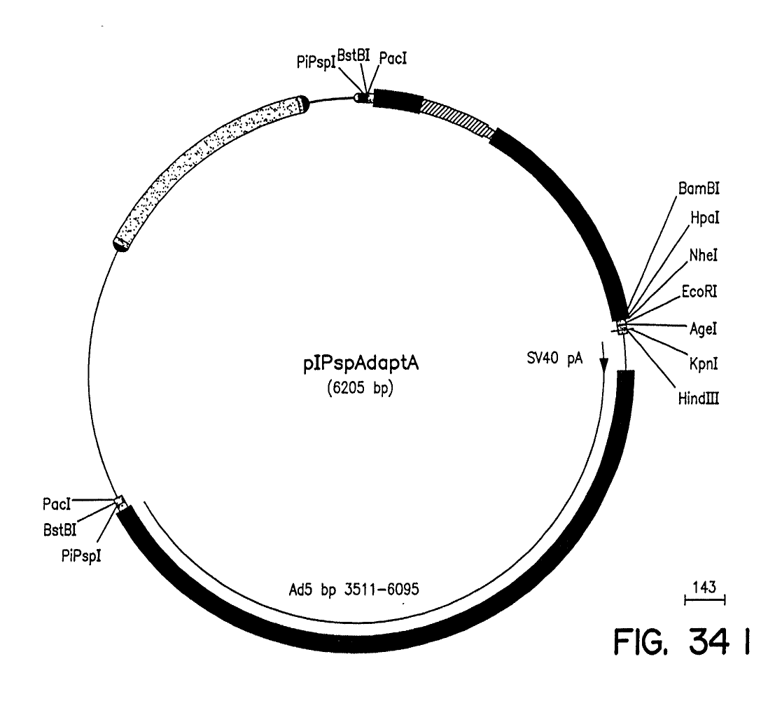

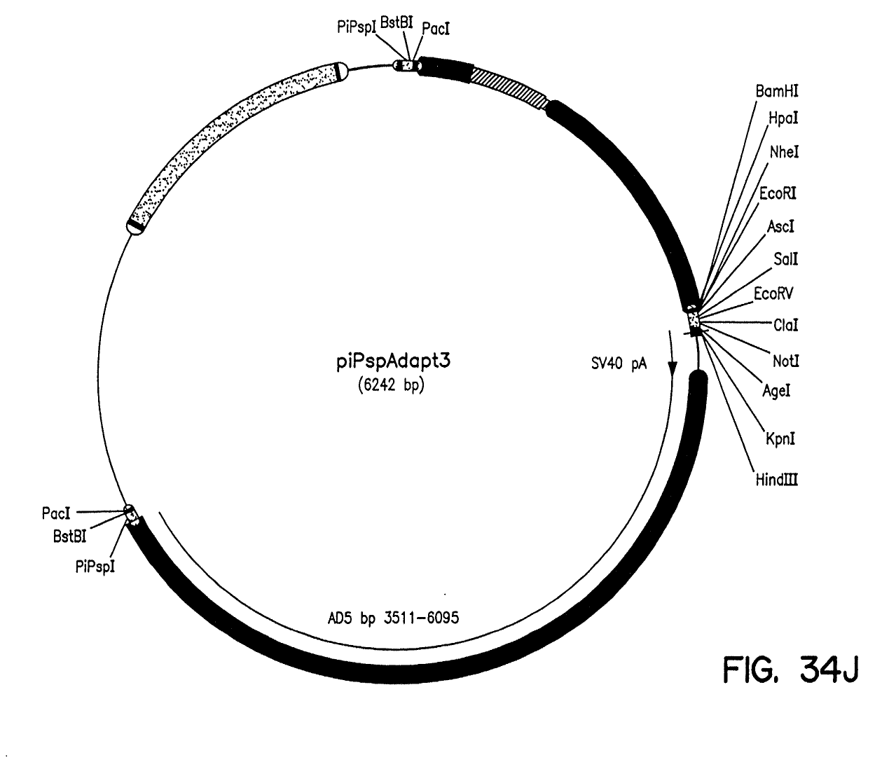

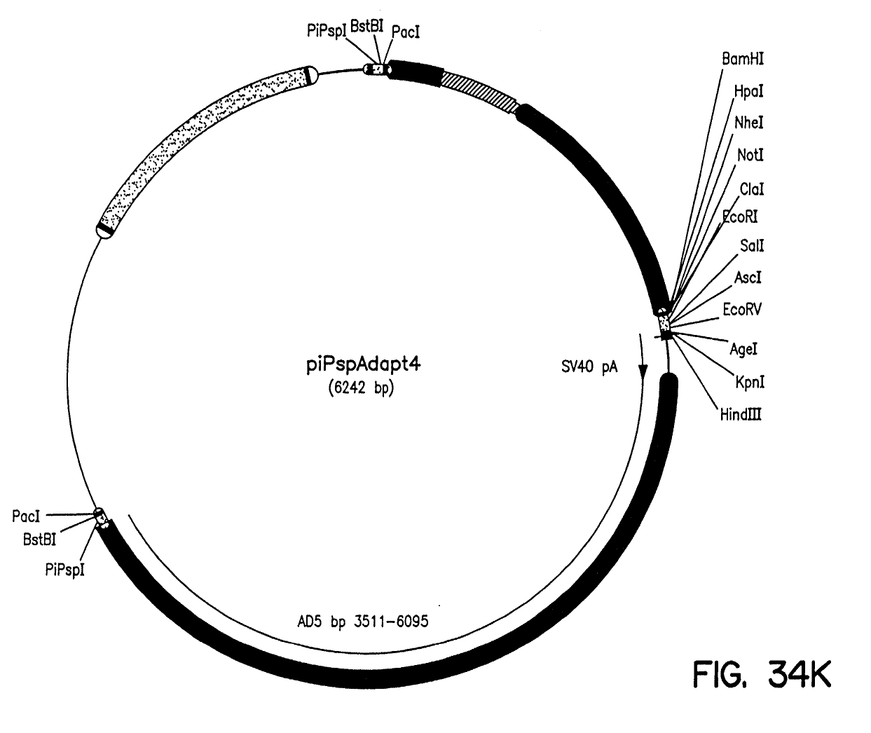

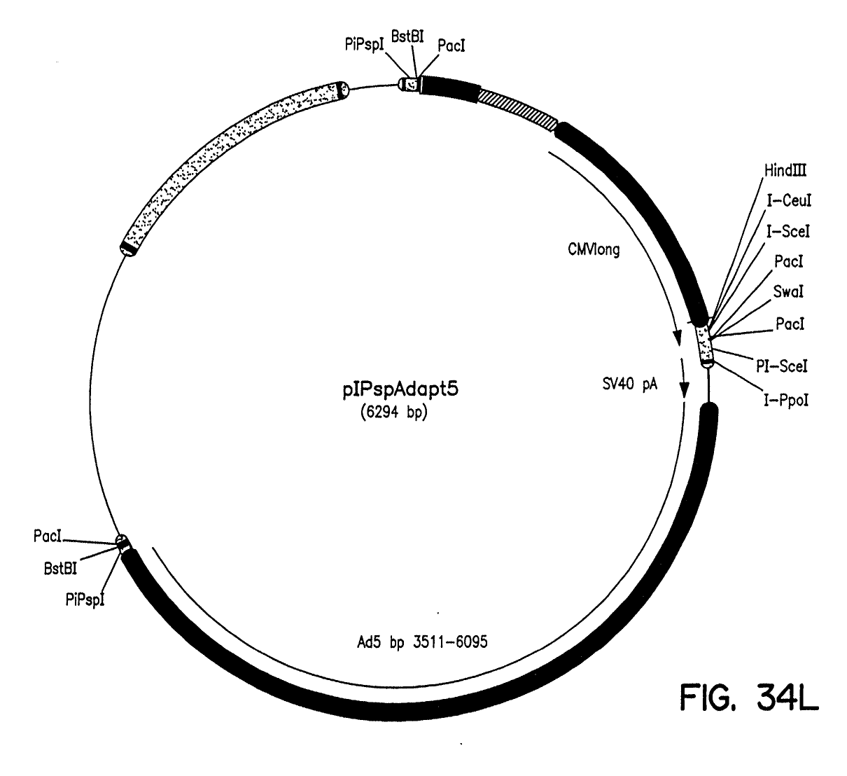

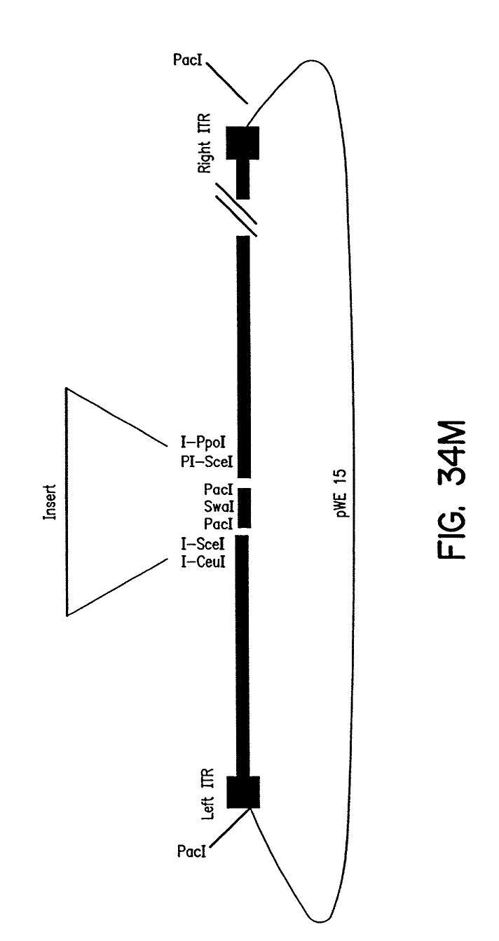

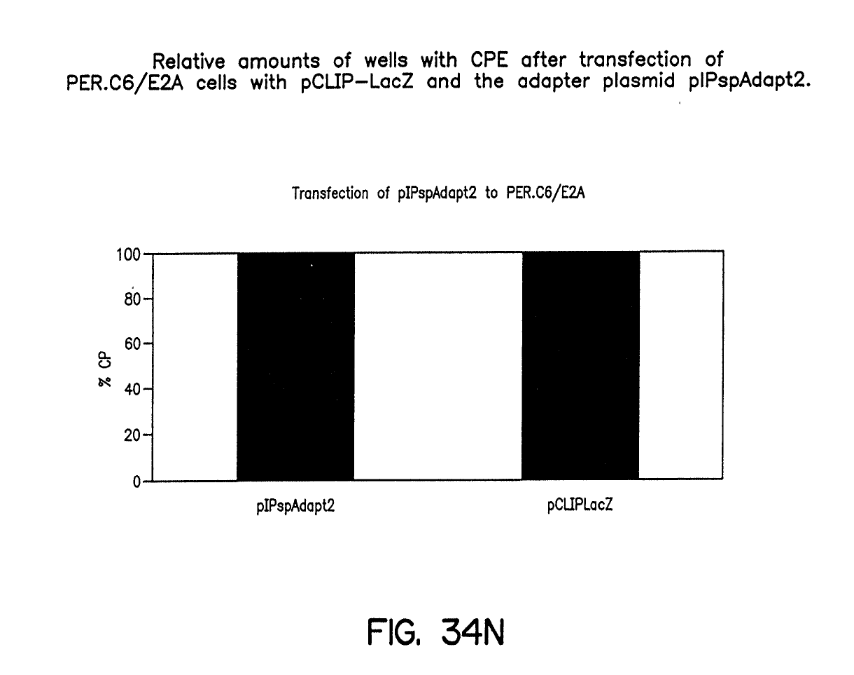

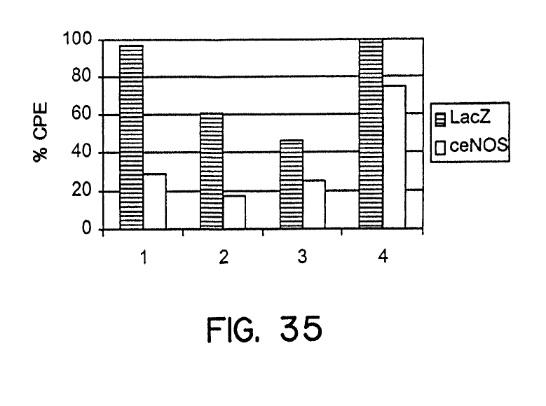

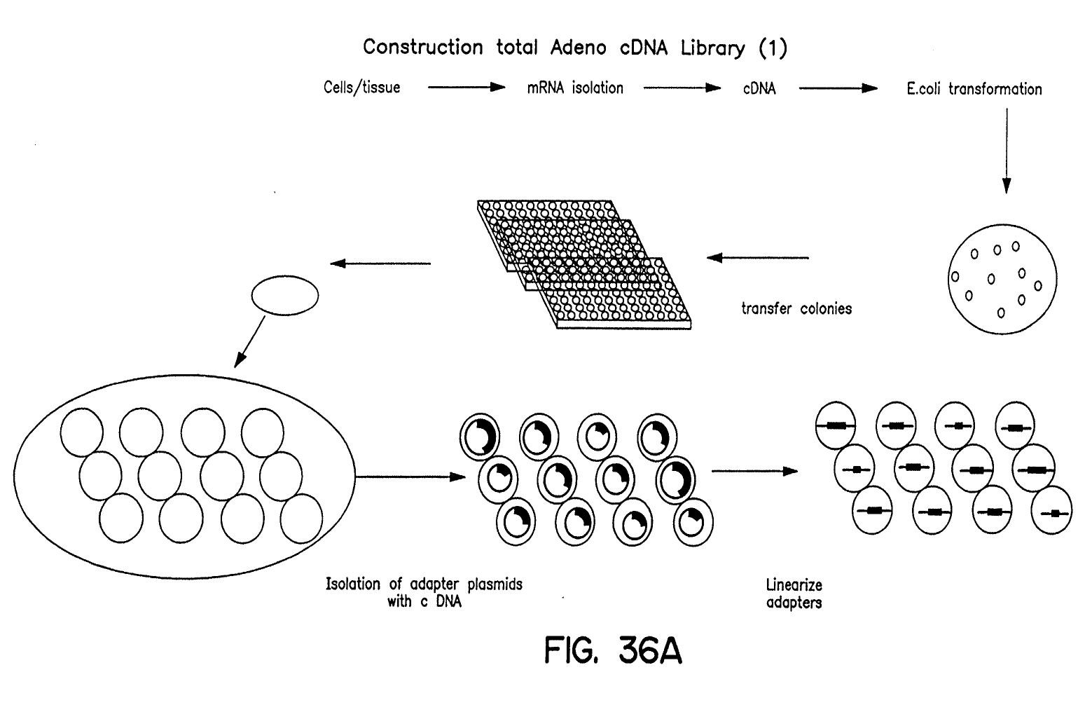

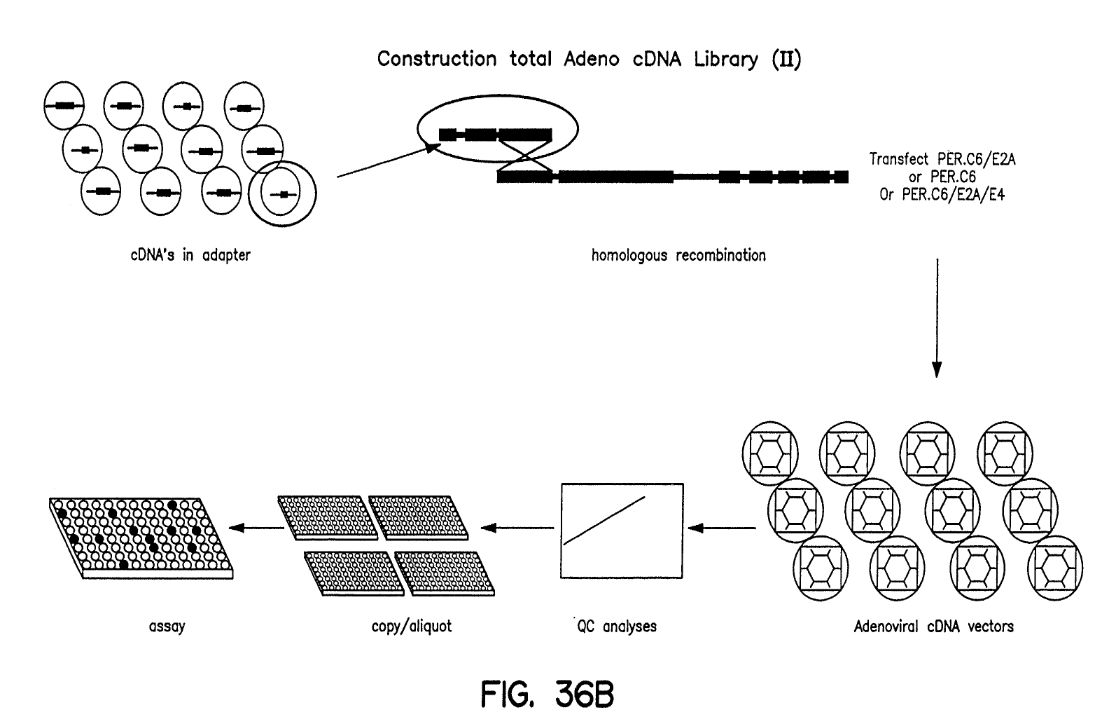

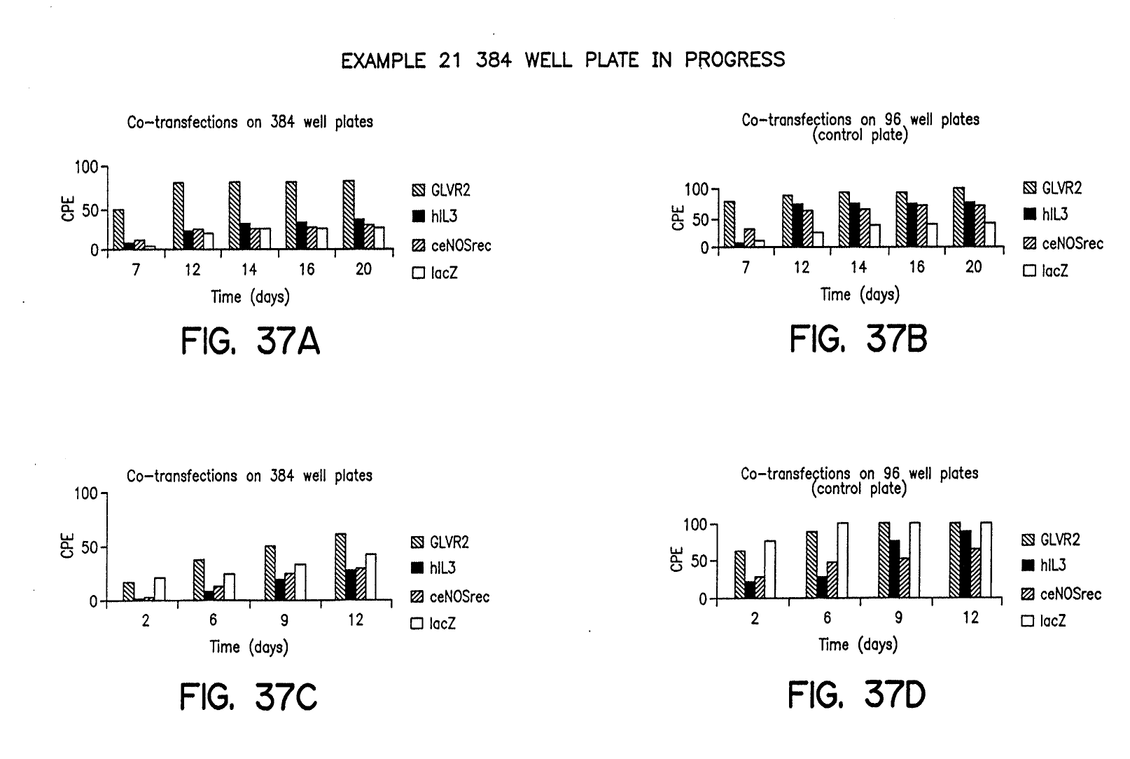

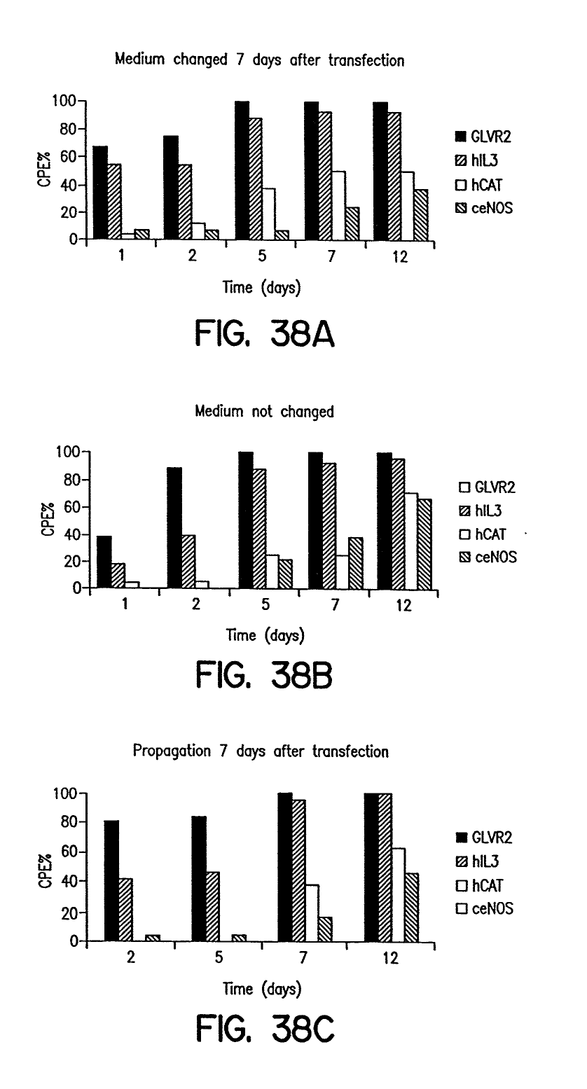

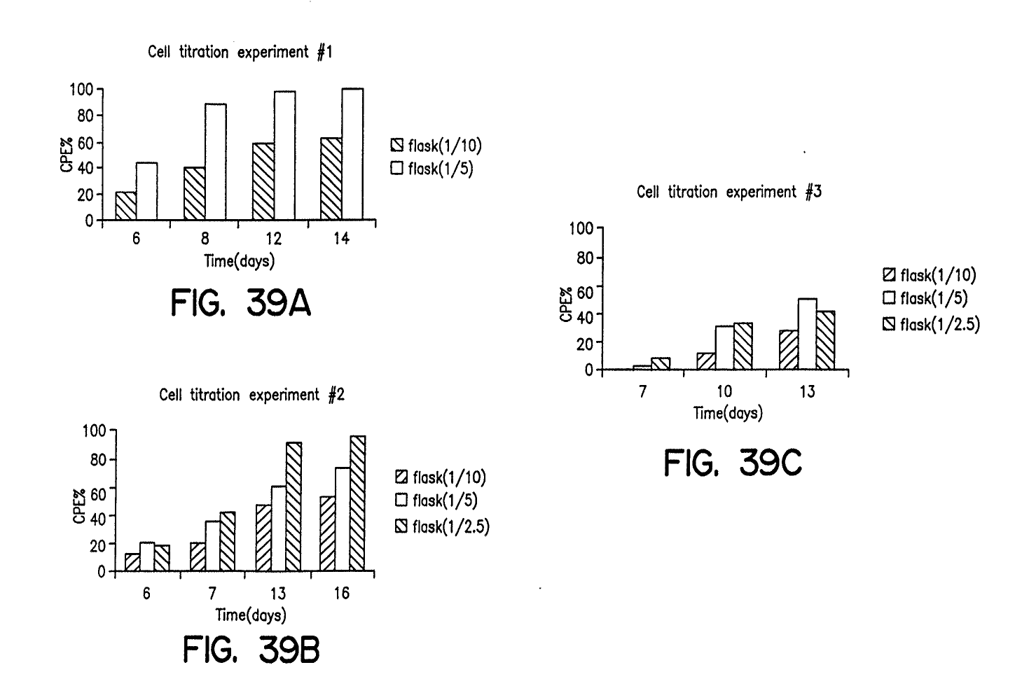

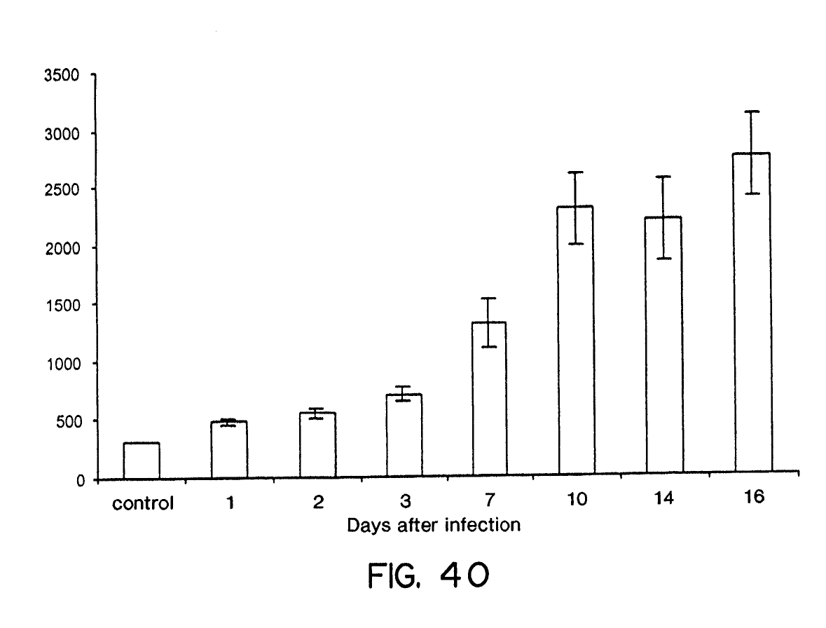

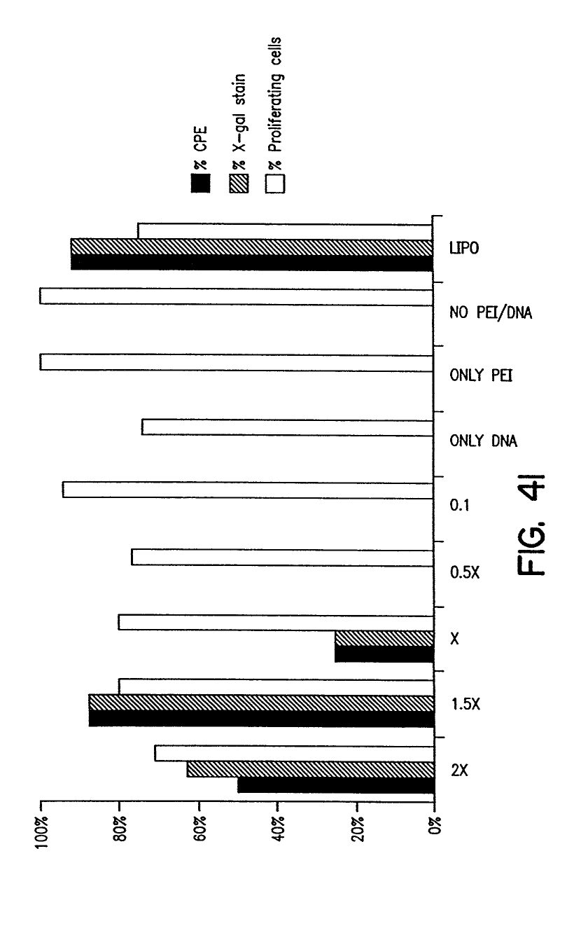

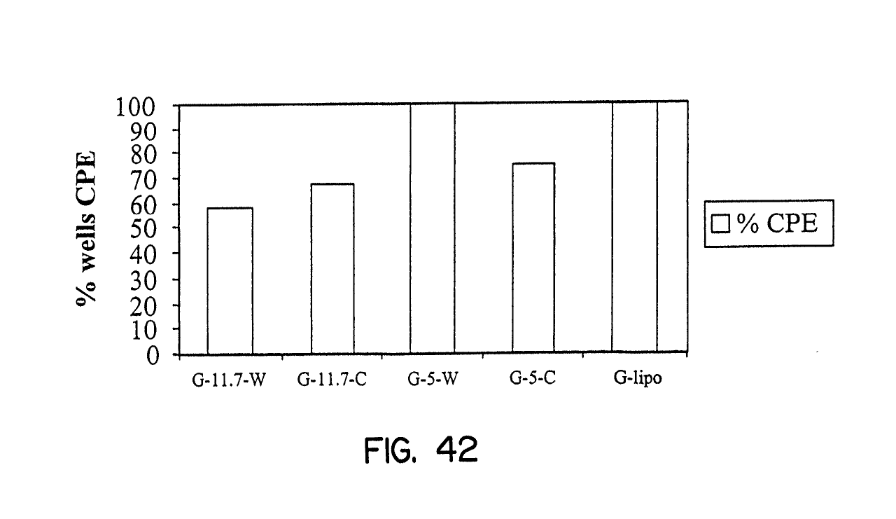

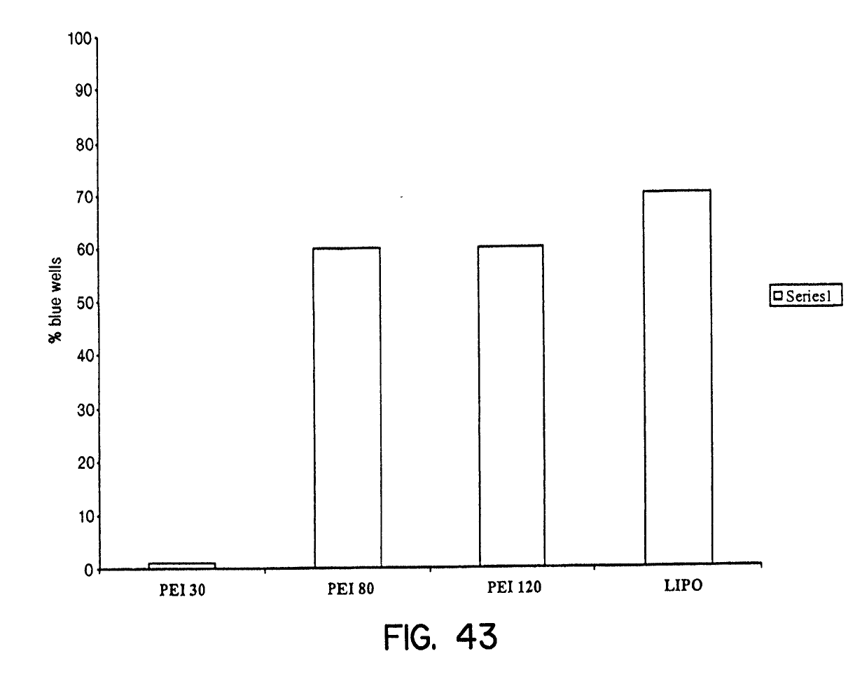

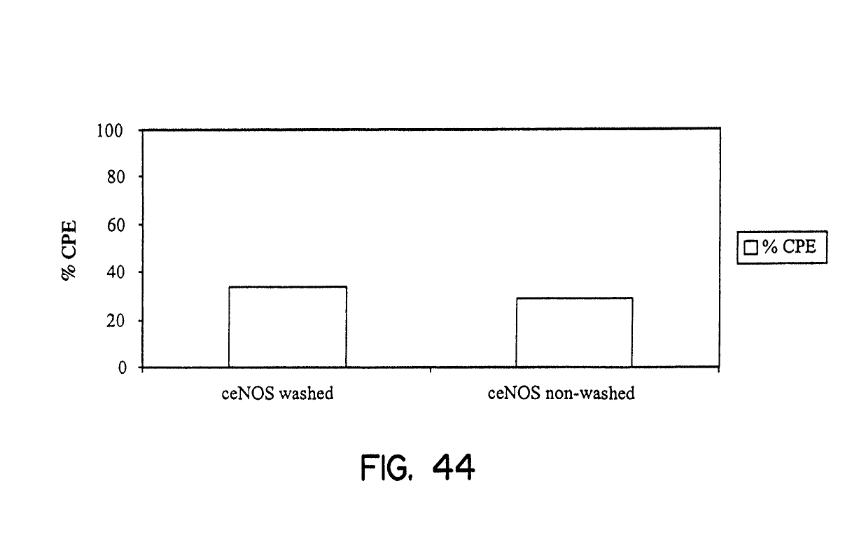

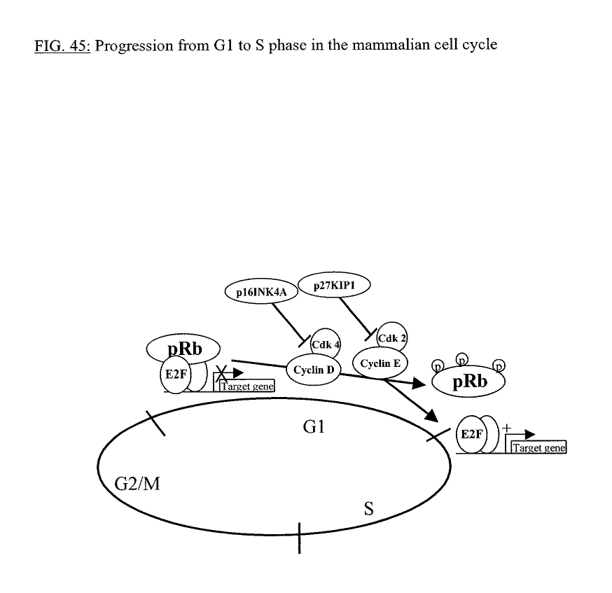

The vector into which the sample nucleic acids are preferably introduced contains, in an operable configuration, an ITR, at least one cloning site or preferably a multiple cloning site for insertion of a library of sample nucleic acids, and transcriptional regulatory elements for delivery and expression of the sample nucleic acids in a host. It generally does not contain E1 region sequences, E2B region sequences (other than those required for late gene expression), E2A region sequences, E3 region sequences, or E4 region sequences. The E1-deleted delivery vector or adapter plasmid is digested with the appropriate restriction enzymes, either simultaneously or sequentially, to produce the appropriate ends for directional cloning of the sample nucleic acid whether it be synthetic double-stranded oligonucleotides, genomic DNA, cDNA, ESTs, or a previously-cloned DNA. Restriction enzyme digestion is routinely performed using commercially available reagents according to the manufacturer's recommendations and varies according to the restriction enzymes chosen. A distinct set or pool of sample nucleic acids is inserted into E1-deleted adapter plasmids to produce a corresponding set or library of plasmids for the production of adenoviral vectors. An example of an adapter plasmid is pMLPI.TK, which is made up of adenoviral nucleotides 1-458 followed by the adenoviral major late promoter, functionally linked to the herpes simplex virus thymidine kinase gene, and followed by adenoviral nucleotides 3511-6095. Other examples of adapter plasmids are pAd/L420-HSA (FIG. 21) and pAd/Clip (FIG. 22). pAd/L420-HSA contains adenoviral nucleotides 1-454, the L420 promoter linked to the murine HSA gene, a poly-A signal, and adenoviral nucleotides 3511-6095. pAd/CLIP is made from pAd/L420-HSA by replacement of the expression cassette (L420-HSA) with the CMV promoter, a multiple cloning site, an intron, and a poly-A signal.

Once digested, the vector and sample nucleic acids are purified by gel electrophoresis. The nucleic acids can be extracted from various gel matrices by, for example, agarose digestion, electroelution, melting, or high salt extraction. In combination with these methods or alternatively, the digested nucleic acids can be purified by chromatography (e.g., Qiagen or equivalent DNA binding resins) or phenol-chloroform extraction followed by ethanol precipitation. The optimal purification method depends on the size and type of the vector and sample nucleic acids. Both can be used without purification. Generally, the sample nucleic acids contain 5′-phosphate groups and the vector is treated with alkaline phosphatase to promote nucleic acid-vector ligation and prevent vector-vector ligation. If the sample nucleic acid is a synthetic oligonucleotide, 5′-phosphate groups are added by chemical or enzymatic means. For ligation, molar ratios of sample nucleic acids (insert) to vector DNA range from approximately 10:1 to 0.1:1. The ligation reaction is performed using T4 DNA ligase or any other enzyme that catalyzes double-stranded DNA ligation. Reaction times and temperature can vary from about 5 minutes to 18 hours, and from about 15° C. to room temperature, respectively.

The magnitude of expression can be modulated using promoters (CMV immediately early, promoter, SV40 promoter, or retrovirus LTRs) that differ in their transcriptional activity. Operatively linking the sample nucleic acid to a strong promoter such as the CMV immediate early promoter and optionally one or more enhancer element(s) results in higher expression allowing the use of a lower multiplicity of infection to alter the phenotype of a host. The option of using a lower multiplicity of infection increases the number of times a library can be used in situ, in vitro, and in vivo. Moreover, the lower the multiplicity of infection and dosages used in screening programs, assays, and animal models decreases the chance of eliciting toxic effects in the transduced host, which increases the reliability of the subject of this invention. Another way to reduce possible toxic effects relating to expression of the library is to use a regulatable promoter, particularly one which is repressed during virus production but can be turned on or is active during functional screenings with the adenoviral library, to provide temporal and/or cell type specific control throughout the screening assay. In this way, sample nucleic acids that are members of the library and are toxic, inhibitory, or in any other way interfere with adenoviral replication and production, can still be produced as an adenoviral vector (see WO 97/20943). Examples of this type of promoter are the AP1-dependent promoters which are repressed by adenoviral E1 gene products, resulting in shut off of sample nucleic acid expression during adenoviral library production (see van Dam, et al. (1990) Mol. Cell. Biol. 10(11):5857-64). Such a promoter can be made using combinatorial techniques or natural or adapted forms of promoters can be included. Examples of AP 1-dependent promoters are promoters from the collagenase, c-myc, monocyte chemoattractant protein (JE or mcp-1/JE), and stromelysin genes (Hagmeyer, et al. (1993) EMBO J. 12(9):3559-72; Offringa, et al. (1990) Cell 62(23):527-38; Offringa, et al. (1988) Nucleic Acids Res. 16(23):10973-84; van Dam, et al. (1989) Oncogene 4(10): 1207-12). Alternatively, other more specific but stronger promoters can be used especially when complex in vitro screenings or in vivo models are employed and tissue-regulated expression is desired. Any promoter/enhancer system functional in the chosen host can be used. Examples of tissue-regulated promoters include promoters with specific activity or enhanced activity in the liver, such as the albumin promoter (Tronche, et al. (1990) Mol. Biol. Med. 7(2): 173-85). Another approach to enhanced expression is to increase the half-life of the mRNA transcribed from the sample nucleic acids by including stabilizing sequences in the 3′ untranslated region. A second nucleic acid construct, a helper plasmid having sequences homologous to sequences in the E1-deleted adapter plasmids, which carries all necessary adenoviral genes necessary for replication and packaging, also is prepared. Preferably, the helper plasmid has no complementing sequences that are expressed by the packaging cells that would lead to production of RCA. In addition, preferably the helper plasmids, adapter plasmid, and packaging cell have no sequence overlap that would lead to homologous recombination and RCA formation. The region of sequence overlap shared between the adapter plasmid and the helper plasmid allows homologous recombination and the formation of an E1-deleted, replication-defective recombinant adenoviral genome. Generally, the region of overlap encompasses E2B region sequences that are required for late gene expression. The amount of overlap that provides for the best efficiency without appreciably decreasing the size of the library insert can be determined empirically. The sequence overlap is generally 10 bp to 5000 bp, more preferably 2000 to 3000 bp.

The size of the sample nucleic acids or DNA inserts in a desired adenoviral library can vary from several hundred base pairs (e.g., sequences encoding neuropeptides) to more than 30 kb (e.g., titin). The cloning capacity of the adenoviral vectors produced using adapter plasmids can be increased by deletion of additional adenoviral gene(s) that are then easily complemented by a derivative of an E1-complementing cell line. As an example, candidate genes for deletion include E2, E3, and/or E4. For example, regions essential for adenoviral replication and packaging are deleted from the adapter and helper plasmids and expressed, for example, by the complementing cell line. For E3 deletions, genes in this region do not need to be complemented in the packaging cell for in vitro models; in in vivo models, the impact upon immunogenicity of the recombinant virus can be assessed on an ad hoc basis.

The set or library of specific adapter plasmids or pool(s) of adapter plasmids is converted to a set or library of adenoviral vectors. The adapter plasmids containing the sample nucleic acids are linearized and transfected into an E1-complementing cell line. The adapter plasmids are preferably seeded in microtiter tissue culture plates with 96, 384, 1,536 or more wells per plate, together with helper plasmids having sequences homologous to sequences in the adapter plasmid and containing all adenoviral genes necessary for replication and packaging. Recombination occurs between the homologous sequences shared by adapter and helper plasmids to generate an E1-deleted, replication-defective adenoviral genome that is replicated and packaged, preferably, in an E1-complementing cell line. If more than one helper plasmid is used, recombination between homologous regions shared among the helper plasmids and recombination between the helper plasmids and adapter plasmid results in the formation of a replication-defective recombinant adenoviral genome. The regions of sequence overlap between the adapter and helper plasmids are at least about a few hundred nucleotides or greater. Recombination efficiency will increase by increasing the size of the overlap.

The E1-functions provided by the trans complementing packaging cell permit the replication and packaging of the E1-deleted recombinant adenoviral genome. The adapter and/or helper plasmids preferably have no sequence overlap amongst themselves or with the complementing sequences in the packaging cells that can lead to the formation of RCA. Preferably, at least one of the ITRs on the adapter and helper plasmids is flanked by a restriction enzyme recognition site not present in the adenoviral sequences or expression cassette so that the ITR is freed from vector sequences by digestion of the DNA with that restriction enzyme. In this way, initiation of replication occurs more efficiently. In order to increase the efficiency of recombinant adenoviral generation, higher throughput can be obtained by using microtiter tissue culture plates with 96, 384, or 1,536-wells per plate instead of using large tissue culture vials or flasks. E1-complementing cell lines are grown in microtiter plates and cotransfected with the libraries of adapter plasmids and a helper plasmid(s). Automation of the method using, for example, robotics can further increase the number of adenoviral vectors that can be produced (Hawkins, et al. (1997) Science 276(5320):1887-9; Houston, (1997) Methods Find. Exp. Clin. Pharmacol. 19 Suppl. A:43-5).

As an alternative to the adapter plasmids, the sample nucleic acids can be ligated to “minimal” adenoviral vector plasmids. The so-called “minimal” adenoviral vectors, according to the present invention, retain at least a portion of the viral genome that is required for encapsidation of the genome into virus particles (the encapsidation signal). The minimal vectors also retain at least one copy of at least a functional part or a derivative of the ITR, that is DNA sequences derived from the termini of the linear adenoviral genome that are required for replication. The minimal vectors preferably are used for the generation and production of helper- and RCA-free stocks of recombinant adenoviral vectors and can accommodate up to 38 kb of foreign DNA. The helper functions of the minimal adenoviral vectors are preferably provided in trans by encapsidation-defective, replication-competent DNA molecules that contain all the viral genes encoding the required gene products, with the exception of those genes that are present in the complementing cell or genes that reside in the vector genome.

Packaging of the “minimal” adenoviral vector is achieved by cotransfection of an E1-complementing cell line with a helper virus or, alternatively, with a packaging deficient replicating helper system. Preferably, the packaging deficient replicating helper is amplified following transfection and expresses the sequences required for replication and packaging of the minimal adenoviral vectors that are not expressed by the packaging cell line. The packaging deficient replicating helper is not packaged into adenoviral particles because its size exceeds the capacity of the adenoviral virion and/or because it lacks a functional encapsidation signal. The packaging deficient replicating helper, the minimal adenoviral vector, and the complementing cell line, preferably, have no region of sequence overlap that permits RCA formation.

The replicating, packaging deficient helper molecule always contains all adenoviral coding sequences that produce proteins necessary for replication and packaging, with or without the coding sequences provided by the packaging cell line. Replication of the helper molecule itself may or may not be mediated by adenoviral proteins and ITRs. Helper molecules that replicate through the activity of adenoviral proteins (for example, E2-gene products acting together with cellular proteins) contain at least one ITR derived from adenovirus. The E2-gene products can be expressed by an E1-dependent or an E1-independent promoter. Where only one ITR is derived from an adenovirus, the helper molecule also preferably contains a sequence that anneals in an intramolecular fashion to form a hairpin-like structure.

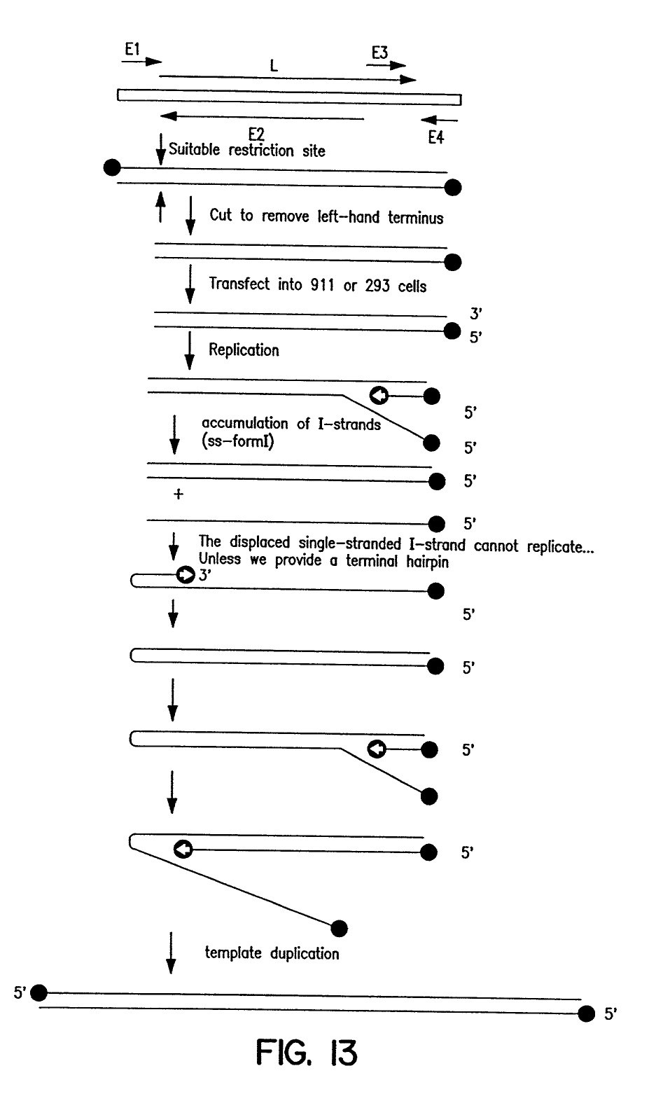

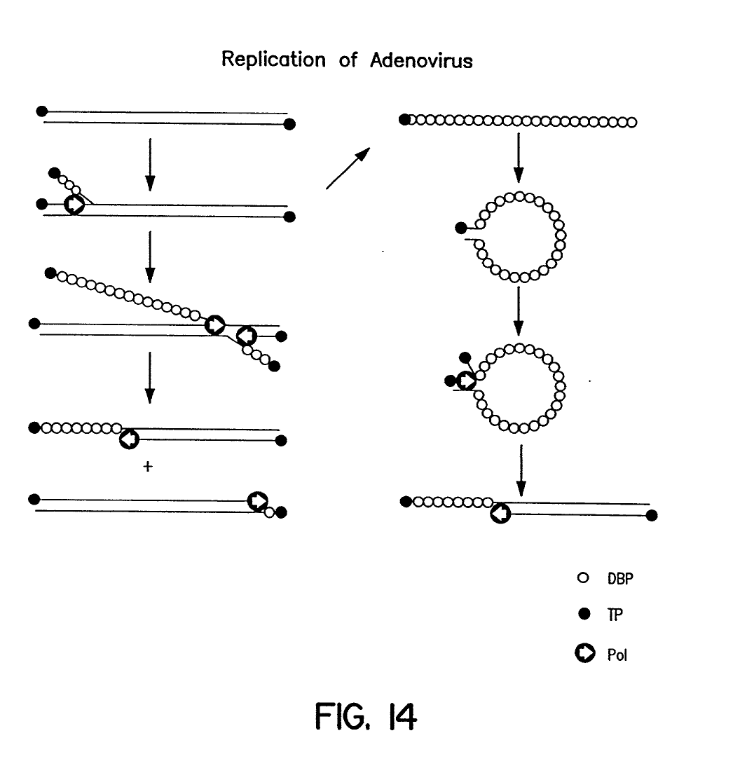

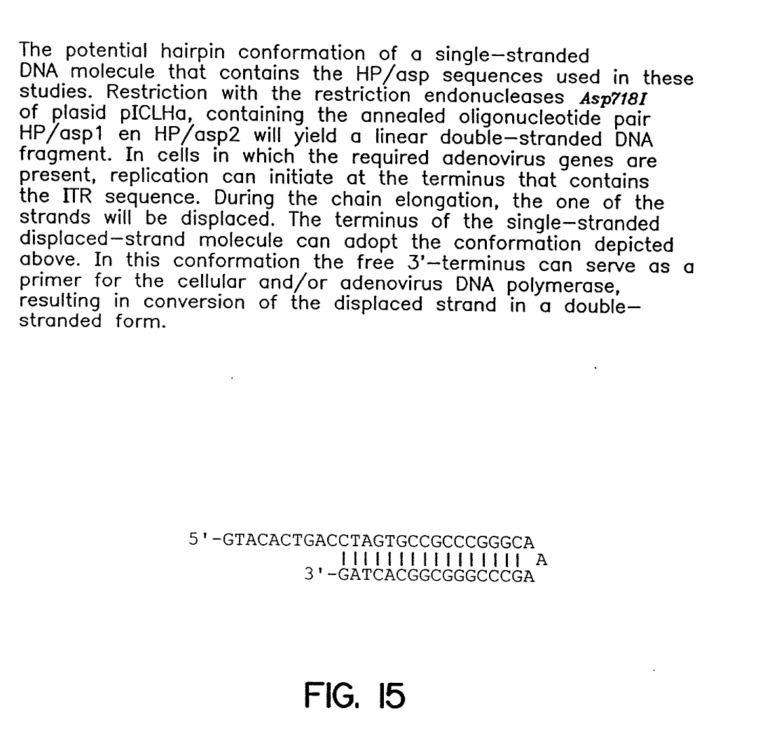

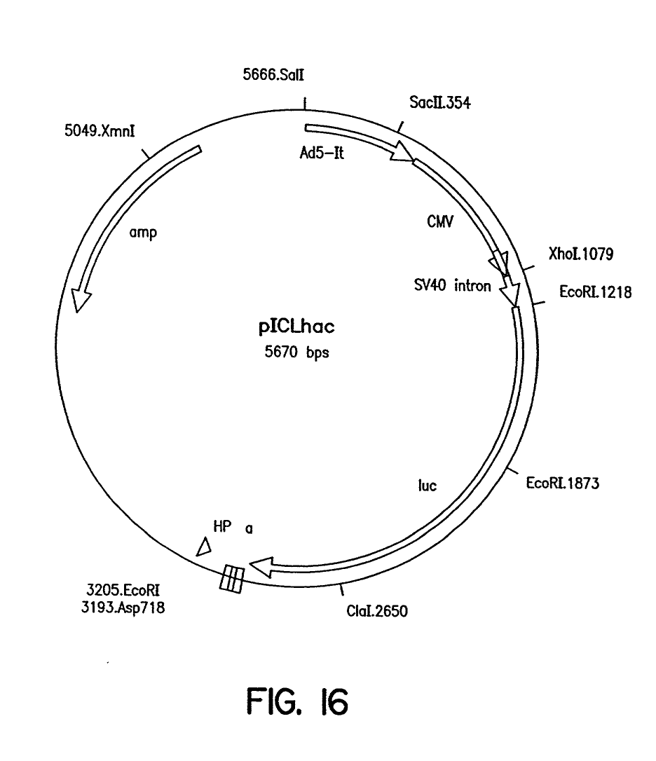

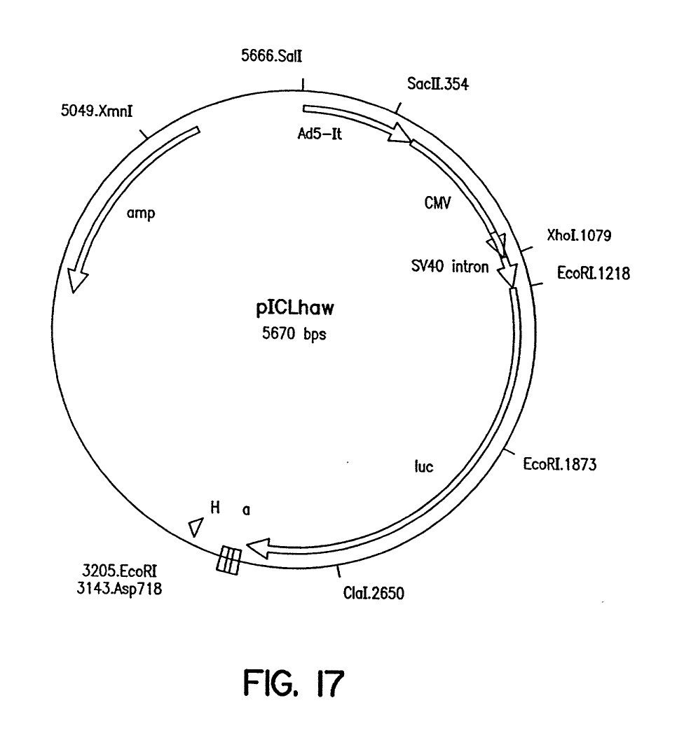

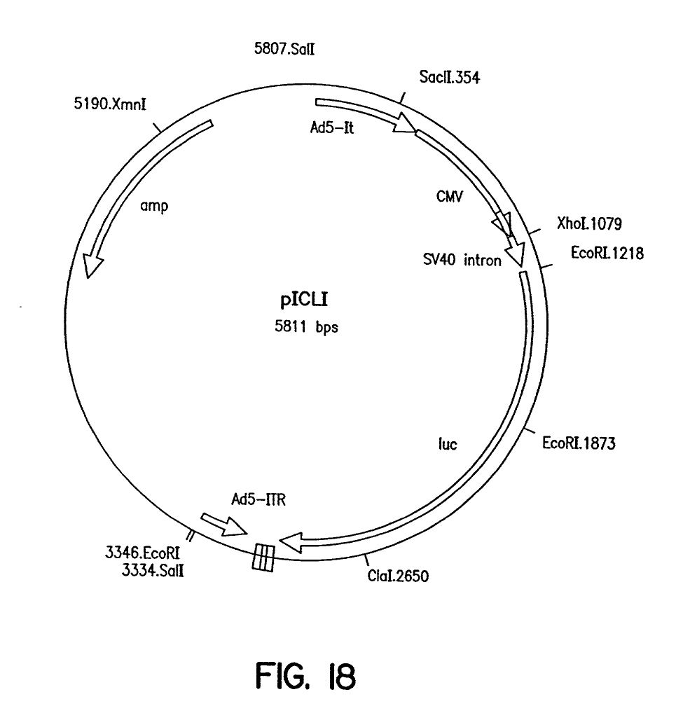

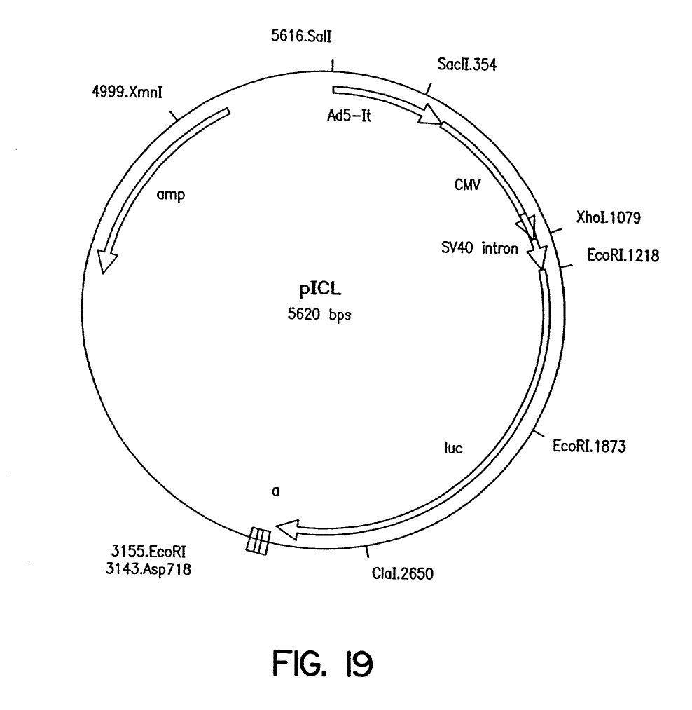

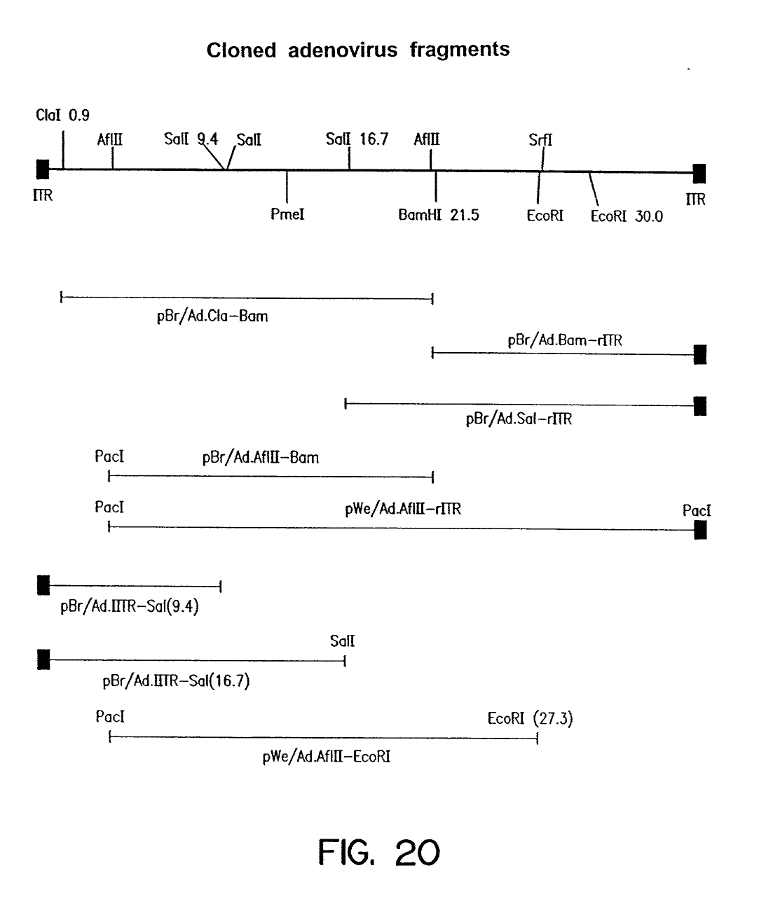

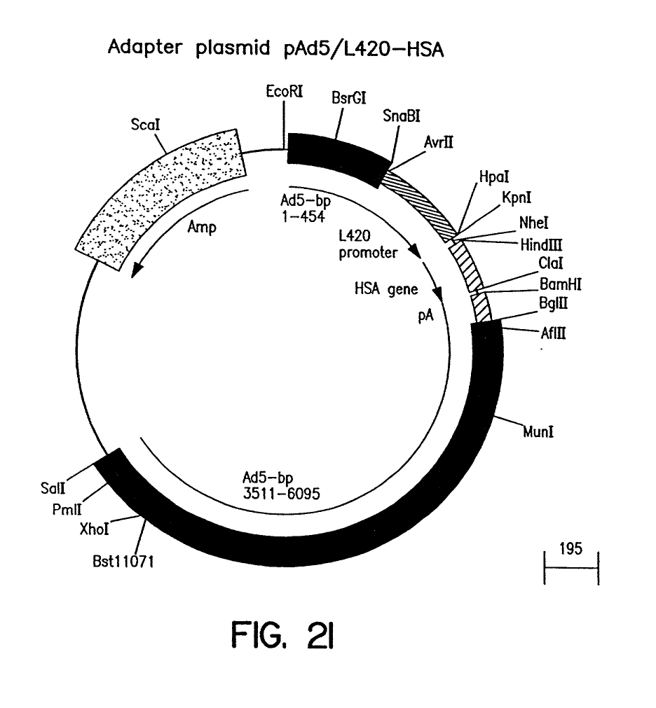

Following E2-gene product expression, the adenoviral DNA polymerase recognizes the ITR on the helper molecule and produces a single-stranded copy. Then, the 3′-terminus intramolecularly anneals, forming a hairpin-like structure that serves as a primer for reverse strand synthesis. The product of reverse strand synthesis is a double-strand hairpin with an ITR at one end. This ITR is recognized by adenoviral DNA polymerase that produces a double-stranded molecule with an ITR at both termini (see e.g., FIG. 13) and becomes twice as long as the transfected molecule (in our example it duplicates from 35 Kb to 70 Kb). Duplication of the helper DNA enhances the production of sufficient levels of adenoviral proteins. Preferably, the size of the duplicated molecule exceeds the packaging capacity of the adenoviral virion and is, therefore, not packaged into adenoviral particles. The absence of a functional encapsidation signal in the helper molecule further ensures that the helper molecule is packaging deficient. The produced adenoviral proteins mediate replication and packaging of the cotransfected or co-infected minimal vectors.

When the replication of the helper molecule is independent of adenoviral E2-proteins, the helper construct preferably contains an origin of replication derived from SV40. Transfection of this DNA, together with the minimal adenoviral vector in an E1-containing packaging cell line that also inducibly expresses the SV40 Large T protein or as much SV40 derived proteins as necessary for efficient replication, leads to replication of the helper construct and expression of adenoviral proteins. The adenoviral proteins then initiate replication and packaging of the co-transfected or co-infected minimal adenoviral vectors. Preferably, there are no regions of sequence overlap shared by the replication-deficient packaging constructs, the minimal adenoviral vectors, and the complementing cell lines that may lead to the generation of RCA.

It is to be understood that during propagation of the minimal adenoviral vectors, each amplification step on E1-complementing cells is preceded by transfection of any of the described helper molecules since minimal vectors by themselves cannot replicate on E1-complementing cells. Alternatively, a cell line that contains all the adenoviral genes necessary for replication and packaging, which are stably integrated in the genome and can be excised and replicated conditionally, can be used (Valerio and Einerhand, International patent Application PCT/NL9800061).

Transfection of nucleic acid into cells is required for packaging of recombinant vectors into virus particles and can be mediated by a variety of chemicals including liposomes, DEAE-dextran, polybrene, and phosphazenes or phosphazene derivatives (WO 97107226). Liposomes are available from a variety of commercial suppliers and include DOTAP® (Boehringer-Mannheim), Tfx®-50, Transfectam®, ProFection® (Promega, Madison, Wis., and LipofectAmine®, Lipofectin®, LipofectAce® (GibcoBRL, Gaithersburg, Md.). In solution, the lipids form vesicles that associate with the nucleic acid and facilitate its transfer into cells by fusion of the vesicles with cell membranes or by endocytosis. Other transfection methods include electroporation, calcium phosphate coprecipitation, and microinjection. If transfection conditions for a given cell line have not been established or are unknown, they can be determined empirically (Maniatis, et al. Molecular Cloning, pages 16.30-16.55).

The yield of recombinant adenoviral virus vectors can be increased by denaturing the double stranded plasmid DNA before transfection into an E1 complementing cell line. Denaturing can occur by heating double-stranded DNA at, for example, 95-100° C., followed by rapid cooling using various ratios of the adapter and helper plasmids that have overlapping sequences. Also, a PER.C6 derivative that stably or transiently expresses E2A (DNA binding protein) and/or E2B gene (pTP-Pol) could be used to increase the adenoviral production per well by increasing the replication rate per cell. Alternatively, cotransfection of recombinase protein(s), recombinase DNA expression construct(s), i.e., recombinase from Kluyveromyces waltii (Ringrose, et al. (1997) Eur. J. Biochem. 248(3):903-12), or any other gene or genes encoding factors that can increase homologous recombination efficiency can be used. The inclusion of 0.1-100 mM sodium butyrate during transfection and/or replication of the packaging cells can increase viral production. These improvements will result in improved viral yields per microtiter well. Therefore, the number and type of assays that can be done with one library will increase and may overcome variability between the various genes or sample nucleic acids in a library.

The cell lines used for the production of adenoviral vectors that express E1 region products includes, for example, 293 cells, PER.C6 (ECACC 96022940), or 911 cells. Each of these cell lines has been transfected with nucleic acids that encode for the adenoviral E1 region. These cells stably express E1 region gene products and have been shown to package E1-deleted recombinant adenoviral vectors. Yields of recombinant adenovirus obtained on PER.C6 cells are higher than obtained on 293 cells.

Each of these cell lines provides the basis for introduction of E2B, E2A, or E4 constructs (e.g., ts125E2A and/or hrE2A) that permit the propagation of adenoviral vectors that have mutations, deletions, or insertions in the corresponding genes. These cells can be modified to express additional adenoviral gene products by the introduction of recombinant nucleic acids that stably express the appropriate adenoviral genes or recombinant nucleic acids and that transiently express the appropriate gene(s), for example, the packaging deficient replicating helper molecules or the helper plasmids.

All (or nearly all) trans complementing cells grown in microtiter plate wells (96, 384, or more than 1,536-wells) produce recombinant adenovirus following transfection with either the adapter plasmid or the minimal adenoviral plasmid library and the appropriate helper molecule(s). A large number of adenoviral gene transfer vectors or a library, each expressing a unique gene, can thus be conveniently produced on a scale that allows analysis of the biological activity of the particular gene products both in vitro and in vivo. Due to the wide tissue tropism of adenoviral vectors, a large number of cell and tissue types are transducible with an adenoviral library.

In one example, growth medium of the cell culture contains sodium butyrate in an amount sufficient to enhance production of the recombinant adenoviral vector library.

Preferably, the plurality of cells further includes at least one of an adenoviral preterminal protein and a polymerase complementing sequence. Preferably, the plurality of cells further includes an adenoviral E2 complementing sequence. Preferably, the E2 complementing sequence is an E2A complementing sequence or an E2B complementing sequence. In one aspect, the plurality of cells further includes a recombinase protein, whereby the homologous recombination leading to replication-defective, recombinant adenovirus is enhanced. Preferably, the recombinase protein is a Kluyveromyces waltii recombinase. In another aspect, the plurality of cells further includes a nucleotide sequence coding for a recombinase protein. Preferably, the recombinase protein is Kluyveromyces waltii recombinase.

Libraries of genes or sample nucleic acids preferably are converted to RCA free adenoviral libraries and used in the present invention in combination with high throughput screening of compounds involving a number of in vitro assays, such as immunological assays including ELISAs, proliferation assays, drug resistance assays, enzyme activity assays, organ cultures, differentiation assays, and cytotoxicity assays. Adenoviral libraries can be tested on tissues, tissue sections, or tissue derived primary short-lived cell cultures including primary endothelial and smooth muscle cell cultures (Wijnberg, et al. (1997) Thromb. Haemost. 78(2):880-6), coronary artery bypass graft libraries (Vassalli, et al. (1997) Cardiovasc. Res. 35(3):459-69; Fuster and Chesebro, (1985) Adv. Prostaglandin Thromboxane Leukot. Res. 13:285-99), umbilical cord tissue including HUVEC (Gimbrone, (1976) Prog. Hemost. Thromb. 3:1-28; Striker, et al. (1980) Methods Cell. Biol. 21A:135-51), couplet hepatocytes (Graf, et al. (1984) Proc. Natl. Acad. Sci. USA 81(20):6516-20), and epidermal cultures (Fabre, (1991) Immunol. Lett. 29(1-2):161-5; Phillips, (1991) Transplantation 51(5):937-41). Plant cell cultures, including suspension cultures, can also be used as host cells for the adenoviral libraries carrying any DNA sequence, including human derived DNA sequences and plant derived sequences. (de Vries, et al. (1994) Biochem. Soc. Symp. 60:43-50; Fukada, et al. (1994) Int. J. Devel. Biol. 38(2):287-99; Jones, (1983) Biochem. Soc. Symp. 48:221-32; Kieran, et al. (1997) J. Biotechnol. 59(1-2):39-52; Stanley, (1993) Curr. Opin. Genet. Dev. 3(l):91-6; Taticek, et al. (1994) Curr. Opin. Biotechnol. 5(2):165-74.

In addition, in vitro assays can be complemented by using an electronic version of the sequence database on which the adenoviral library is built. This allows, for example, protein motif searching whereby new members of a family can be linked to known members of the same family with known functions. The use of Hidden Markow Models (HMMs) (Eddy, (1996) Proc. Natl. Acad. Sci. USA 94(4):1414-9) allows the establishment of novel families by identifying essential features of a family and building a model of what the members should look like. This can be combined with structural data by using the threading approach, which uses a known structure as the thread and tries to find a putative structure without having determined the actual structure of the novel protein (Rastan and Beeley, (1997) Curr. Opin. Genet. Dev. 7(6):777-83). The functional data, which is obtained using adenoviral libraries made in accordance with the methods disclosed in this application, is the foundation of the endeavor to find novel genes with expected or desired functions and will be the core of functional genomics. Finally, once the number of adenoviral vectors has reached a level at which animal experiments can be performed, another addition to the method is to produce the selection of candidate adenoviral vectors carrying the candidate genes. Then, the clones can be purified by, for example, using adenovirus tagged in the Hi loop of the knob domain of the fiber. Alternatively, large scale HPLC analysis can be used in a semipreparative fashion to yield partially purified adenoviral samples for in vivo or in vitro experiments when more purified adenoviral preparations are desired. Therefore, the described method and reagents allow rapid transfer of a collection of genes in in vivo studies of a limited number of animals, which otherwise would be unfeasible. The automation of the steps of the procedure using robotics will further enhance the number of genes and sample nucleic acids that can be functionated.

Aspects of the present invention include methods of assay and compositions used therein for the identification of compounds useful for the treatment of disease states that involve apoptosis-associated processes. The methods and compositions of the present invention are based on the identification of the polypeptides and polynucleotides discovered by the adenoviral library screening methods described hereinabove. Examples of polynucleotides and polypeptides identified by the methods of the present invention are the polynucleotide of SEQ ID NO: 13, polypeptides comprising an amino acid sequence encoded by the polynucleotide of SEQ ID NO: 13 and the polypeptide comprising the amino acid sequence of SEQ ID NO: 14. The invention includes both naturally occurring and recmbinant forms of SEQ ID NO: 13 and SEQ ID NO: 14 as well as methods of their production. Methods of detecting the polynucleotide of SEQ ID NO: 13 include probing with polynucleotides complementary to SEQ ID NO: 13 (northern and southern hybridization) and amplifying using the polymerase chain reaction. Methods of detection of the polypeptide of SEQ ID NO: 14 and polypeptides encoded by SEQ ID NO: 14 inlcude the use of epitope tags as well as immunodetectioon.

By using these polypeptides and polynucleotides as targets in screening assays, such as high throughput screens, small molecule compounds can be identified as drug candidates for pharmaceutical development. As will be discussed in a subsequent section herein below, the present invention also relates pharmaceutical compositions and methods of treatment comprising these polypeptides and polynucleotides.

High Throughput Binding Screen for Compounds that Affect the Ability of the Identified Genes to Alter E2F activity

Screening assays for drug candidates are designed to identify compounds that bind or complex with the polypeptides encoded by the genes identified herein, or otherwise interfere with the interaction of the encoded polypeptides with other cellular proteins. Such screening assays will include assays amenable to high-throughput screening of chemical libraries, making them particularly suitable for identifying small molecule drug candidates. Small molecules contemplated include synthetic organic or inorganic compounds, including peptides, preferably soluble peptides, (poly)peptide-immunoglobulin fusions, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. The assays can be performed in a variety of formats, including protein-protein binding assays, biochemical screening assays, immunoassays and cell based assays, which are well characterized in the art.

Isolated antibodies that specifically bind to a polypeptide of SEQ ID NO: 14 or a polypeptide encoded by a polynucleotide of SEQ ID NO: 13 can be generated by methods known in the art and screened as drug candidates. Types of antibodies include, but are not limited to polyclonal antibodies, monoclonal antibodies, chimeric antibodies, single chain antibodies, FAb fragments, F(ab)₂ fragments, and humanized antibodies.

Assays involve the contacting, under conditions and for a time sufficient to allow interaction, of the drug candidate with a polypeptide or a polynucleotide that alters E2F activity. In binding assays, the interaction is binding and the complex formed can be isolated or detected in the reaction mixture. In a particular embodiment, the polypeptide or polynucleotide that alters E2F activity or the drug candidate is immobilized on a solid phase, e.g., on a microtiter plate, by covalent or non-covalent attachments. Non-covalent attachment generally is accomplished by coating the solid surface with a solution of the polypeptide or polynucleotide and drying. Alternatively, an immobilized antibody, e.g., a monoclonal antibody, specific for the polypeptide or polynucleotide to be immobilized can be used to anchor it to a solid surface. The assay is performed by adding the non-immobilized component, which may be labelled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labelled antibody specifically binding the immobilized complex. If the candidate compound interacts with but does not bind to a polypeptide or polynucleotide that alters E2F activity, its interaction with that molecule can be assayed by methods well known for detecting interactions. Such assays include traditional approaches, such as, cross-linking, co-immunoprecipitation, and co-purification through gradients or chromatographic columns.

To screen for antagonists and/or agonists of gene products identified herein, the assay mixture is incubated under conditions whereby, but for the presence of the candidate pharmacological agent, the identified gene product alters E2F activity. The mixture components can be added in any order that provides for the requisite activity. Incubation may be performed at any temperature that facilitates optimal binding, typically between about 4° C. and 40° C., more commonly between about 15° C. and 40° C. Incubation periods are likewise selected for optimal binding but also minimized to facilitate rapid, high-throughput screening, and are typically between about 0.1 and 10 hours, preferably less than 5 hours, more preferably less than 2 hours. After incubation, the effect of the candidate pharmacological agent is determined in any convenient way. For cell-free binding-type assays, a separation step is often used to separate bound and unbound components. Separation may, for example, be effected by precipitation (e.g., TCA precipitation, immunoprecipitation, etc.), immobilization (e.g., on a solid substrate), followed by washing. The bound protein is conveniently detected by taking advantage of a detectable label attached to it, e.g., by measuring radioactive emission, optical or electron density, or by indirect detection using, e.g., antibody conjugates.

Suitable compounds that bind to the polypeptide or polynucleotide include polypeptide or polynucleotide fragments or small molecules, e.g. peptidomimetics. Such compounds prevent interaction and proper complex formation. Small molecule compounds, which are usually less than 10 kD molecular weight, are preferable as therapeutics since they are more likely to be permeable to cells, are less susceptible to degradation by various cellular mechanisms, and are not as apt to elicit an immune response as would proteins or polypeptides. Small molecules include but are not limited to synthetic organic or inorganic compounds. Many pharmaceutical companies have extensive libraries of such molecules, which can be conveniently screened by using the assays of the present invention. Non-limiting examples include proteins, peptides, glycoproteins, glycopeptides, glycolipids, polysaccharides, oligosacchardies, nucleic acids, bioorganic molecules, peptidomimetics, pharmacological agents and their metabolites, transcriptional and translation control sequences, and the like.

A preferred technique for identifying compounds that bind to the polypeptide or polynucleotide utilizes a chimeric substrate (e.g., epitope-tagged fused or fused immunoadhesin) attached to a solid phase, such as the well of an assay plate. The binding of the candidate molecules, which are optionally labelled (e.g., radiolabelled), to the immobilized receptor can be measured.

The invention further discloses methods for assessing toxicity of a test compound, said method comprising treating a biological sample containing nucleic acids with the test compound; hybridizing the nucleic acids of the treated biological sample with a probe comprising at least 20 contiguous nucleotides of a polynucleotide of SEQ ID NO: 13 under conditions whereby a specific hybridization complex is formed between said probe and a target polynucleotide in the biological sample, said target polynucleotide comprising a polynucleotide sequence of a polynucleotide of SEQ ID NO: 13 or fragment thereof; quantifying the amount of hybridization complex; and comparing the amount of hybridization complex in the treated biological sample with the amount of hybridization complex in an untreated biological sample, wherein a difference in the amount of hybridization complex in the treated biological sample is indicative of toxicity of the test compound.

The invention further discloses arrays, including microarrays, comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, said target polynucleotide having a sequence of SEQ ID NO: 13.

Identification of Antagonists of E2F Activity

The present method identifies compounds useful in abrogation of E2F activity by selecting test compounds that exhibit binding affinity to a polynucleotide comprising a sequence of SEQ ID NO: 13. The determination of binding affinities of such test compounds for the present polynucleotides employs in vitro assay methods known in the art. The most preferred test compounds also selectively bind the polynucleotides of the present invention.

In a preferred method, test compounds that exhibit binding affinity are contacted with a first subpopulation of host cells transfected with the polynucleotide for which the test compound has affinity. The host cells are preferably primary cells, more preferably human primary cells, and most preferably HUVEC cells. The host cells are transfected with the polynucleotide using methods known in the art, for example, as described above in connection with the adenoviral vectors transfection.

A second subpopulation of transfected host cells is not contacted with the test compound exhibiting binding affinity and is used as a control.

The first and second subpopulations of cells are then examined for E2F activity to determine if E2F activity has been altered in the first subpopulation relative to the second control subpopulation. E2F activity may be detected by a variety of methods known in the art, including expression of a reporter gene operably linked to multiple E2F binding sites. A reporter sequence is “operably linked” to a transcription factor binding site (e.g., E2F binding site) when the transcription factor is capable of directing transcription of the reporter sequence upon binding of the transcription factor to the transcription factor binding site. Reporter genes include, but are not limited to, genes encoding for luciferase, EGFP, Renilla luciferase, and alkaline phosphatase. Compounds that alter E2F activity are candidates for pharmaceutical development as anti-proliferative or anti-apoptotic drugs.

A further method for identifying a compound useful in the treatment of apoptosis-associated disorders selects test compounds that exhibit binding affinity to a polypeptide comprising a sequence of SEQ ID NO: 14. The assay methods are similar to those described above, except that the target is the polypeptide in contrast to the polynucleotide. The host cells are transfected with an expression vector encoding the polynucleotide that encodes the polypeptide using methods known in the art. The expression vector may be any suitable expression vector that can express the polypeptide in the host cell. Preferred expression vectors include adenoviral vectors described herein to transfect such cells.

As in the foregoing assay description, a second subpopulation of transfected host cells is not contacted with the test compound exhibiting binding affinity, and is used as a control. The first and second subpopulations of cells are then examined for E2F activity to determine if E2F activity has been altered in the first subpopulation relative to the second control subpopulation.

In an alternative method for identifying such drug compounds, one or more test compounds are contacted with a corresponding number of one or more subpopulations of host cells transfected with an expression vector encoding a polynucleotide identified in the library screening methods. Examples of such polynucleotides to be used in this assay include a polynucleotide comprising a sequence of SEQ ID NO: 13. The host cells may be any of the host cell types used in the methods described above. The transfection may be performed using methods known in the art. Compounds that alter E2F activity in the first subpopulation of cells that have been transfected (or transduced) with the expression vector relative to a second subpopulation of host cells that have not been contacted with a test compound, are selected as drug candidates for pharmaceutical development for the treatment of apoptosis-associated disorders.

Another method for identifying drug candidate compounds is based on the measurement, in the cellular mRNA population of the host cells, of mRNA encoded by the polynucleotide comprising a sequence of SEQ ID NO: 13. The level of mRNA expression can be measured by a variety of methods known in the art. A drug candidate compound may be selected by comparing the mRNA expression level in the first subpopulation of host cells relative to expression of the mRNA in a second subpopulation of host cells that have not been contacted with a test compound. A decrease in the mRNA expression of the above-referenced polynucleotide would identify a compound candidate for pharmaceutical development for the treatment of apoptosis-associated disorders.

Identification of Test Compounds that Bind to SEQ ID NOS: 13 or 14

The present method identifies compounds useful in the treatment of apoptosis-associated disorders by selecting test compounds that exhibit binding affinity to a polynucleotide comprising a sequence of SEQ ID NO: 13 or to a polypeptide comprising a sequence of SEQ ID NO: 14.

One such method is based on polypeptide binding and contacts a test compound with a polypeptide identified in the above-described adenoviral library screening methods. Examples of such polypeptides include SEQ ID NO: 14.

The binding affinity of the test compounds for the polypeptide is then determined using methods known in the art. The binding affinity may be in a nanomolar to micromolar concentrations, with nanomolar concentration preferred.

A further aspect of this method contacts a test compound that exhibits binding affinity to the target polypeptide with a first subpopulation of host cells. The host cells may be any cells that allow activation of E2F. Preferred cells include immortal cells, such as neoplastic cells. Drug candidate compounds are selected from test compounds that bind to the aforesaid polypeptide and that induce an increase in expression of mRNA corresponding to a polynucleotide comprising a sequence of SEQ ID NO: 13 in the first subpopulation relative to expression of mRNA in a second subpopulation of host cells that has not been contacted with the test compound.

Another aspect of the present method comprises the contacting of a test compound that exhibits binding affinity for the polypeptide with a first subpopulation of host cells transfected with an expression vector encoding such polypeptide. Such first subpopulation of host cells is examined to determine if E2F activity is enhanced in the first subpopulation relative to a second subpopulation that is not contacted with such compound. Alternatively, the first subpopulation of host cells may be transfected with a lower MOI than used in the adenoviral library assay method described above, for example, using an MOI lower than that used in the library screening method. The method can be adapted using an MOI titration to determine the activity of the test compound. Exemplary MOIs can range from 0-10%, 10-20%, 20-50% of the standard MOI. By using an MOI that is insufficient to induce E2F activity in the transfected subpopulation of host cells, the present method is capable of a more sensitive determination of compounds that induce E2F activity.

Compounds that exhibit binding affinity for the polypeptide and enhance E2F activity in the first subpopulation of host cells treated with said compound relative to a control untreated subpopulation of host cells are selected as drug candidate compounds. The control subpopulation of host cells is preferably transfected using the same MOI as the first subpopulation of host cells.

In another aspect of the present invention, one or more test compounds are contacted with a corresponding number of one or more first subpopulations of host cells transfected with an expression vector encoding a polynucleotide identified in the library screening methods. Examples of expression vectors to be used include expression vectors comprising a polynucleotide sequence of SEQ ID NO: 13. The test compounds in accordance with this method may or may not have been previously identified as having any binding affinity to the aforesaid polypeptides or polynucleotides.

A drug candidate compound is selected from those compounds that enhance E2F activity in the first subpopulation of host cells relative to a second subpopulation of host cells that have not been contacted with such compound. In an alternative aspect of the present invention, a drug candidate compound is selected from those compounds that induce an increase in expression of mRNA encoded by a polynucleotide identified using the above-described library screening method in a first subpopulation of cells relative to expression of said mRNA in a second subpopulation of host cells that has not been contacted with such test compound. The preferred mRNA populations measured in this method are encoded by a polynucleotide comprising a sequence of SEQ ID NO: 13. The level of expression of mRNA can be measured by a variety of methods known in the art.

In a further aspect of this method, a third population of cells comprising primary cells are contacted with test compounds that exhibit binding affinity to said target polypeptide or polynucleotide. Test compounds that alter E2F activity in the neoplastic host cells and are not toxic to the primary cells are selected preferentially. Such compounds are candidates for drug development. A particularly preferred drug candidate comprises compounds that induce apoptosis in the neoplastic host cells and that do no affect the primary cell hosts.

Depending on the size of the initial unselected library, once an adenoviral library of genes has been reduced to a reasonable number of candidates by in vitro assays, the adenoviruses can be tested in appropriate animal models. Examples of animal models that can be used include models for Alzheimer's disease, arteriosclerosis, cancer metastasis, and Parkinson's disease. In addition, transgenic animals which have altered expression of endogenous or exogenous genes including mice with gene(s) that have been inactivated, animals with cancers implanted at specific sites, human bone marrow chimeric mice such as NOD-SCID mice, and the like can be used. As additional testing is required, the stocks of candidate adenoviruses can be increased by pass aging the adenoviruses under the appropriate transcomplementing conditions. Depending on the animal model used, adenoviral vectors or mixtures of pre-selected pools of adenoviral vectors can be applied or administered at appropriate sites such as lung in non-human primates (Sene, et al. (1995) Hum. Gene Ther. 6(12):1587-93) and brain of normal and apoE deficient mice (Robertson, et al. (1998) Neuroscience 82(1): 171-80.) for Alzheimer's disease (Walker, et al. (1997) Brain Res. Brain Res. Rev. 25(l):70-84) and Parkinson disease models (Hockman, et al. (1971) Brain Res. 35(2):613-8; Zigmond and Stricker, (1984) Life Sci. 35(1):5-18). The adenoviral vectors or mixtures of pre-selected pools of adenoviral vectors can also be injected in the blood stream for liver disease models including liver failure and Wilson disease (Cuthbert, (1995) J. Investig. Med. 43(4):323-36; Karrer, et al. (1984) Curr. Surg. 41(6):464-7) and tumor models including metastases models (Esandi, et al. (1997) Gene Ther. 4(4):280-7; Vincent, et al. (1996) J. Neurosurg. 85(4):648-54; Vincent, et al. (1996) Hum. Gene Ther. 7(2): 197-205). In addition, selected adenoviral vectors can be injected directly into the bone marrow of human chimeric NOD-SCID mice (Dick, et al. (1997) Stem Cells 15 Suppl. 1: 199-203; Mosier, et al. (1988) Nature 335(6187):256-9). Finally, selected adenovirus can be applied locally, for example, in vascular tissue of restenosis animal models (Karas, et al. (1992) J. Am. Coll. Cardiol. 20(2):467-74).

In the present invention, a variety of well known animal models of apoptosis-associated disorders can be used to test the efficacy of the drug candidate compounds, including the polypeptides, nucleic acids, antibodies, and agonists and antagonists of the target molecules. The in vivo nature of such models makes them particularly predictive of responses in human patients. Animal models include both non-recombinant and recombinant (transgenic) animals. Non-recombinant animal models include, for example, rodent, e.g., murine models. Examples of animal models that exhibit the apoptosis-associated condition and that are useful in testing the efficacy of candidate therapeutic agents are described hereafter.

Recombinant (transgenic) animal models can be engineered by introducing the coding portion of the genes identified herein into the genome of animals of interest, using standard techniques for producing transgenic animals. A transgenic animal is one containing a “transgene” or genetic material integrated into the genome introduced into the animal itself or an ancestor of the animal at a prenatal stage (e.g., embryonic stage). Animals that can serve as a target for transgenic manipulation include, without limitation, mice, rats, rabbits, guinea pigs, sheep, goats, pigs, and non-human primates, e.g., baboons, chimpanzees and monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten, et al. (1985) Proc. Natl. Acad. Sci. USA 82:6148-52); gene targeting in embryonic stem cells (Thompson, et al. (1989) Cell 56:313-21); electroporation of embryos (Lo, (1983) Mol. Cell. Biol. 3:1803-14); sperm-mediated gene transfer (Lavitrano, et al. (1989) Cell 57:717-73). For review, see, for example, U.S. Pat. No. 4,736,866 and U.S. Pat. No. 4,870,009.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lakso, et al. (1992) Proc. Natl. Acad. Sci. USA 89(14):6232-36.

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals are further examined for signs of tumor or cancer development.

Alternatively, “knock out” animals can be constructed which have a defective or altered gene encoding gene identified in the screen, as a result of homologous recombination between the endogenous gene encoding the gene and altered genomic DNA encoding the same polypeptide introduced into an embryonic cell of the animal. For example, cDNA encoding an identified gene can be used to clone genomic DNA encoding that polypeptide in accordance with established techniques. A portion of the genomic DNA encoding an identified gene can be deleted or replaced with another gene, such as a gene encoding a selectable marker that can be used to monitor integration. Typically, several kilobases of unaltered flanking DNA (both at the 5′ and 3′ ends) are included in the vector (see e.g., Thomas and Capecchi, (1987) Cell 51(3):503-12) for a description of homologous recombination vectors). The vector is introduced into an embryonic stem cell line (e.g., by electroporation) and cells in which the introduced DNA has homologously recombined with the endogenous DNA are selected (see e.g., Li, et al. (1992) Cell 69(6): 915-26). The selected cells are then injected into a blastocyst of an animal (e.g., a mouse or rat) to form aggregation chimeras (see e.g., Bradley, (1987) in Teratocarcinomas and Embryonic Stem Cells: A Practical Approach, E. J. Robertson, ed. IRL, Oxford, 113-1521). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, by their ability to defend against certain pathological conditions and by their development of pathological conditions due to absence of the identified gene.

It may be advantageous to produce nucleic sequences possessing a substantially different codon usage, e.g., inclusion of non-naturally occurring codons from the codons present in a nucleic acid sequence identified using the methods of the present invention. Codons may be selected to increase the rate at which expression of the peptide occurs in a particular prokaryotic or eukaryotic host in accordance with the frequency with which particular codons are utilized by the host. Other reasons for substantially altering a nucleotide sequence without altering the encoded amino acid sequences include the production of RNA transcripts having more desirable properties, such as a greater half-life, than transcripts produced from the naturally occurring sequence.

The invention also encompasses production of DNA sequences that encode derivatives or fragments of the polypeptide encoded by the nucleic acid sequence identified using the methods of the present invention, entirely by synthetic chemistry. After production, the synthetic sequence may be inserted into any of the many available expression vectors and cell systems using reagents well known in the art. Moreover, synthetic chemistry may be used to introduce any desired mutations.

Also encompassed by the invention are polynucleotide sequences that are capable of hybridizing to the claimed polynucleotide sequences, and, in particular, to those shown in SEQ ID NO: 13, and fragments thereof under various conditions of stringency (See, e.g., Wahl and Berger, (1987) Methods Enzymol. 152:399-407; Kimmel, (1987) Methods Enzymol. 152:507-11.) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide.

Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed.

In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200g/ml ssDNA. Useful variations on these conditions will be readily apparent to those skilled in the art.

The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations of these conditions are readily apparent to those skilled in the art.

Polynucleic Acids Identified by the Present Invention

The present invention further relates to the polynucleotides identified in the practice of the method invention described hereinabove, more particularly, those isolated nucleic acids found capable of altering E2F activity. For example, the polynucleotides having the sequences of SEQ ID NO: 13 comprise polynucleotides of the present invention.

The present invention also utilizes antisense nucleic acids that can be used to down-regulate or block the expression of polypeptides capable of altering E2F activity in vitro, ex vivo, or in vivo. The down regulation of gene expression using antisense nucleic acids can be achieved at the translational or transcriptional level. Antisense nucleic acids of the invention are preferably nucleic acid fragments capable of specifically hybridizing with all or part of a nucleic acid encoding a polypeptide capable of altering E2F activity or the corresponding messenger RNA. In addition, antisense nucleic acids may be designed or identified which decrease expression of the nucleic acid sequence capable of altering E2F activity by inhibiting splicing of its primary transcript. With knowledge of the structure and partial sequence of a nucleic acid capable of altering E2F activity, such antisense nucleic acids can be designed and tested for efficacy.

The antisense nucleic acids are preferably oligonucleotides and may consist entirely of deoxyribo-nucleotides, modified deoxyribonucleotides, or some combination of both. The antisense nucleic acids can be synthetic oligonucleotides. The oligonucleotides may be chemically modified, if desired, to improve stability and/or selectivity. Since oligonucleotides are susceptible to degradation by intracellular nucleases, the modifications can include, for example, the use of a sulfur group to replace the free oxygen of the phosphodiester bond. This modification is called a phosphorothioate linkage. Phosphorothioate antisense oligonucleotides are water soluble, polyanionic, and resistant to endogenous nucleases. In addition, when a phosphorothioate antisense oligonucleotide hybridizes to its target site, the RNA-DNA duplex activates the endogenous enzyme ribonuclease (RNase) H, which cleaves the mRNA component of the hybrid molecule.

In addition, antisense oligonucleotides with phosphoramidite and polyamide (peptide) linkages can be synthesized. These molecules should be very resistant to nuclease degradation. Furthermore, chemical groups can be added to the 2′ carbon of the sugar moiety and the 5 carbon (C-5) of pyrimidines to enhance stability and facilitate the binding of the antisense oligonucleotide to its target site. Modifications may include 2′ deoxy, O-pentoxy, O-propoxy, O-methoxy, fluoro, methoxyethoxy phosphoro-thioates, modified bases, as well as other modifications known to those of skill in the art.

Antisense nucleic acids can be prepared by expression of all or part of a sequence selected from the group consisting of SEQ ID NO: 13, in the opposite orientation. Any length of antisense sequence is suitable for practice of the invention so long as it is capable of down-regulating or blocking expression of a nucleic acid capable of altering E2F activity. Preferably, the antisense sequence is at least about 20 nucleotides in length. The preparation and use of antisense nucleic acids, DNA encoding antisense RNAs and the use of oligo and genetic antisense is known in the art.

One approach to determining the optimum fragment of a nucleic acid sequence capable of altering E2F activity in an antisense nucleic acid treatment method involves preparing random cDNA fragments of a nucleic acid capable of altering E2F activity by mechanical shearing, enzymatic treatment, and cloning the fragment into any of the vector systems described herein. Individual clones or pools of clones are used to infect cells expressing the polypeptide and effective antisense cDNA fragments are identified by monitoring expression at the RNA or protein level.

A variety of viral-based systems, including retroviral, adeno-associated viral, and adenoviral vector systems may all be used to introduce and express antisense nucleic acids in cells. Antisense synthetic oligonucleotides may be introduced into the body of a patient in a variety of ways, as discussed below.

Reductions in the levels of polypeptides capable of altering E2F activity may be accomplished using ribozymes. Ribozymes are catalytic RNA molecules (RNA enzymes) that have separate catalytic and substrate binding domains. The substrate binding sequence combines by nucleotide complementarity and, possibly, nonhydrogen bond interactions with its target sequence. The catalytic portion cleaves the target RNA at a specific site. The substrate domain of a ribozyme can be engineered to direct it to a specified mRNA sequence. The ribozyme recognizes and then binds a target mRNA through complementary base-pairing. Once it is bound to the correct target site, the ribozyme acts enzymatically to cut the target mRNA. Cleavage of the mRNA by a ribozyme destroys its ability to direct synthesis of the corresponding polypeptide. Once the ribozyme has cleaved its target sequence, it is released and can repeatedly bind and cleave at other mRNAs.

Ribozyme forms include a hammerhead motif, a hairpin motif, a hepatitis delta virus, group I intron or RNaseP RNA (in association with an RNA guide sequence) motif or Neurospora VS RNA motif. Ribozymes possessing a hammerhead or hairpin structure are readily prepared since these catalytic RNA molecules can be expressed within cells from eukaryotic promoters (Chen, et al. (1992) Nucleic Acids Res. 20:4581-9). A ribozyme of the present invention can be expressed in eukaryotic cells from the appropriate DNA vector. If desired, the activity of the ribozyme may be augmented by its release from the primary transcript by a second ribozyme (Ventura, et al. (1993) Nucleic Acids Res. 21:3249-55).

Ribozyme may be chemically synthesized by combining an oligodeoxyribonucleotide with a ribozyme catalytic domain (20 nucleotides) flanked by sequences that hybridize to the target mRNA after transcription. The oligodeoxyribonucleotide is amplified by using the substrate binding sequences as primers. The amplification product is cloned into a eukaryotic expression vector.

Ribozymes are expressed from transcription units inserted into DNA, RNA, or viral vectors. Transcription of the ribozyme sequences are driven from a promoter for eukaryotic RNA polymerase I (pol I, RNA polymerase II (pol II), or RNA polymerase II (pol III). Transcripts from pol II or pol III promoters will be expressed at high levels in all cells; the levels of a given pol II promoter in a given cell type will depend on nearby gene regulatory sequences. Prokaryotic RNA polymerase promoters are also used, providing that the prokaryotic RNA polymerase enzyme is expressed in the appropriate cells (Gao and Huang, (1993) Nucleic Acids Res. 21:2867-72). It has been demonstrated that ribozymes expressed from these promoters can function in mammalian cells (Kashani-Sabet, et al. (1992) Antisense Res. Dev. 2:3-15).

To express the ribozyme of the present invention, the ribozyme sequence of the present invention is inserted into a plasmid DNA vector, a retrovirus vector, an adenovirus DNA viral vector or an adeno-associated virus vector. DNA may be delivered alone or complexed with various vehicles. The DNA, DNA/vehicle complexes, or the recombinant virus particles are locally administered to the site of treatment, as discussed below. Preferably, recombinant vectors capable of expressing the ribozymes are locally delivered as described below, and persist in target cells. Once expressed, the ribozymes cleave the target mRNA.

Ribozymes may be administered to a patient by a variety of methods. They may be added directly to target tissues, complexed with cationic lipids, packaged within liposomes, or delivered to target cells by other methods known in the art. Localized administration to the desired tissues may be done by catheter, infusion pump or stent, with or without incorporation of the ribozyme in biopolymers. Alternative routes of delivery include, but are not limited to, intravenous injection, intramuscular injection, subcutaneous injection, aerosol inhalation, oral (tablet or pill form), topical, systemic, ocular, intraperitoneal and/or intrathecal delivery. Detailed descriptions of ribozyme delivery and administration are provided in Sullivan et al. WO 94/02595.

The present invention also relates to methods for expressing a polypeptide or polynucleotide identified as capable of altering E2F activity as a gene therapeutic. Preferably, the viral vectors used in the gene therapy methods of the present invention are replication defective. Such replication defective vectors will usually lack at least one region that is necessary for the replication of the virus in the infected cell. These regions can either be eliminated (in whole or in part), or be rendered non-functional by any technique known to a person skilled in the art. These techniques include the total removal, substitution, partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome, which are necessary for encapsidating, the viral particles.

Certain embodiments of the present invention use retroviral vector systems. Retroviruses are integrating viruses that infect dividing cells, and their construction is known in the art. Retroviral vectors can be constructed from different types of retrovirus, such as, MoMuLV (“murine Moloney leukemia virus” MSV (“murine Moloney sarcoma virus”), HaSV (“Harvey sarcoma virus”); SNV (“spleen necrosis virus”); RSV (“Rous sarcoma virus”) and Friend virus. Lentivirus vector systems may also be used in the practice of the present invention.

In other embodiments of the present invention, adeno-associated viruses (“AAV”) are utilized. The AAV viruses are DNA viruses of relatively small size that integrate, in a stable and site-specific manner, into the genome of the infected cells. They are able to infect a wide spectrum of cells without inducing any effects on cellular growth, morphology or differentiation, and they do not appear to be involved in human pathologies.

In the vector construction, the polynucleotides of the present invention may be linked to one or more regulatory regions. Selection of the appropriate regulatory region or regions is a routine matter, within the level of ordinary skill in the art. Regulatory regions include promoters, and may include enhancers, suppressors, etc.

Promoters that may be used in the expression vectors of the present invention include both constitutive promoters and regulated (inducible) promoters. The promoters may be prokaryotic or eukaryotic depending on the host. Among the prokaryotic (including bacteriophage) promoters useful for practice of this invention are lac, lacZ, T3, T7, lambda Pᵣ, Pₗ, and trp promoters. Among the eukaryotic (including viral) promoters useful for practice of this invention are ubiquitous promoters (e.g., HPRT, vimentin, actin, tubulin), intermediate filament promoters (e.g., desmin, neurofilaments, keratin, GFAP), therapeutic gene promoters (e.g., MDR type, CFTR, factor VIII), tissue-specific promoters (e.g., actin promoter in smooth muscle cells, or Flt and Flk promoters active in endothelial cells), including animal transcriptional control regions, which exhibit tissue specificity and have been utilized in transgenic animals: elastase I gene control region which is active in pancreatic acinar cells (Swift, et al. (1984) Cell 38:639-46; Ornitz, et al. (1986) Cold Spring Harbor Symp. Quant. Biol. 50:399-409; MacDonald, (1987) Hepatology 7:425-515); insulin gene control region which is active in pancreatic beta cells (Hanahan, (1985) Nature 315:115-22), immunoglobulin gene control region which is active in lymphoid cells (Grosschedl, et al. (1984) Cell 38:647-58; Adames, et al. (1985) Nature 318:533-8; Alexander, et al. (1987) Mol. Cell. Biol. 7:1436-44), mouse mammary tumor virus control region which is active in testicular, breast, lymphoid and mast cells (Leder, et al. (1986) Cell 45:485-95), albumin gene control region which is active in liver (Pinkert, et al. (1987) Genes and Devel. 1:268-76), alpha-fetoprotein gene control region which is active in liver (Krumlauf, et al. (1985) Mol. Cell. Biol., 5:1639-48; Hammer, et al. (1987) Science 235:53-8), alpha 1-antitrypsin gene control region which is active in the liver (Kelsey, et al. (1987) Genes and Devel., 1:161-71), beta-globin gene control region which is active in myeloid cells (Mogram, et al. (1985) Nature 315:338-40; Kollias, et al. (1986) Cell 46:89-94), myelin basic protein gene control region which is active in oligodendrocyte cells in the brain (Readhead, et al. (1987) Cell 48:703-12), myosin light chain-2 gene control region which is active in skeletal muscle (Sani, (1985) Nature 314:283-6), and gonadotropic releasing hormone gene control region which is active in the hypothalamus (Mason, et al. (1986) Science 234:1372-8).

Other promoters which may be used in the practice of the invention include promoters which are preferentially activated in dividing cells, promoters which respond to a stimulus (e.g., steroid hormone receptor, retinoic acid receptor), tetracycline-regulated transcriptional modulators, cytomegalovirus immediate-early, retroviral LTR, metallothionein, SV-40, E1a, and MLP promoters.

Additional vector systems include the non-viral systems that facilitate introduction of DNA encoding the polypeptides capable of altering E2F activity, the polynucleotides encoding these polypeptides, or antisense nucleic acids into a patient. For example, a DNA vector encoding a desired sequence can be introduced in vivo by lipofection. Synthetic cationic lipids designed to limit the difficulties encountered with liposome mediated transfection can be used to prepare liposomes for in vivo transfection of a gene encoding a marker (Felgner, et. al. (1987) Proc. Natl. Acad. Sci. USA 84:7413-7); see Mackey, et al. (1988) Proc. Natl. Acad. Sci. USA 85:8027-31; Ulmer, et al. (1993) Science 259:1745-8). The use of cationic lipids may promote encapsulation of negatively charged nucleic acids, and also promote fusion with negatively charged cell membranes (Felgner and Ringold, (1989) Nature 337:387-8). Particularly useful lipid compounds and compositions for transfer of nucleic acids are described in International Patent Publications WO 95/18863 and WO 96/17823, and in U.S. Pat. No. 5,459,127. The use of lipofection to introduce exogenous genes into the specific organs in vivo has certain practical advantages and directing transfection to particular cell types would be particularly advantageous in a tissue with cellular heterogeneity, for example, pancreas, liver, kidney, and the brain. Lipids may be chemically coupled to other molecules for the purpose of targeting. Targeted peptides, e.g., hormones or neurotransmitters, and proteins for example, antibodies, or non-peptide molecules could be coupled to liposomes chemically. Other molecules are also useful for facilitating transfection of a nucleic acid in vivo, for example, a cationic oligopeptide (e.g., International Patent Publication WO 95/21931), peptides derived from DNA binding proteins (e.g., International Patent Publication WO 96/25508), or a cationic polymer (e.g. International Patent Publication WO 95/21931).

It is also possible to introduce a DNA vector in vivo as a naked DNA plasmid (see U.S. Pat. Nos. 5,693,622, 5,589,466 and 5,580,859). Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wilson, et al. (1992) J. Biol. Chem. 267:963-7; Wu and Wu, (1988) J. Biol. Chem. 263:14621-4; Hartmut, et al. Canadian Patent Application No. 2,012,311, filed Mar. 15, 1990; Williams, et al (1991). Proc. Natl. Acad. Sci. USA 88:2726-30). Receptor-mediated DNA delivery approaches can also be used (Curiel, et al. (1992) Hum. Gene Ther. 3:147-54; Wu and Wu, (1987) J. Biol. Chem. 262:4429-32).

Polypeptides Identified by the Present Invention

The present invention also relates to the polypeptides, or subfragments thereof, which have been identified by the practice of the present method invention as capable of altering E2F activity. Such polypeptides include for example, the polypeptides that are encoded by nucleic acids, including, for example, SEQ ID NO: 14, or which comprise antibodies capable of binding to such polypeptides encoded by such nucleic acids.

The polypeptides of the present invention may be prepared by recombinant technology methods, isolated from natural sources, or prepared synthetically, and may be of human, or other animal origin. The polypeptides of the present invention may be unglycosylated or modified subsequent to translation. Such modifications include glycosylation, phosphorylation, acetylation, myristoylation, methylation, isoprenylation, and palmitoylation. Preferred glycosylated polypeptides are produced in mammalian cells, and most preferably in human cells, a particular embodiment of which are the PER.C6 cells. Using recombinant DNA technology, the nucleic acid encoding the polypeptide is inserted into a suitable vector, which is inserted into a suitable host cell. The polypeptide produced by the resulting host cell is recovered and purified. The polypeptides are characterized by amino acid composition and sequence, and biological activity. Other ways to characterize the polypeptides include reproducible single molecular weight and/or multiple set of molecular weights, chromatographic response and elution profiles,

The present invention also provides antibodies directed against polypeptides capable of altering E2F activity. These antibodies may be monoclonal antibodies or polyclonal antibodies. The present invention includes chimeric, single chain, and humanized antibodies, as well as FAb fragments and the products of an FAb expression library, and Fv fragments and the products of an Fv expression library.

In certain embodiments, polyclonal antibodies may be used in the practice of the invention. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the identified gene product or a fusion protein thereof. Antibodies may also be generated against the intact protein or polypeptide, or against a fragment, derivative, or epitope of the protein or polypeptide, by using for example a library of antibody variable regions, such as a phage display library.

It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). The immunization protocol may be selected by one skilled in the art without undue experimentation.

In some embodiments, the antibodies may be monoclonal antibodies. Monoclonal antibodies may be prepared using methods known in the art. The monoclonal antibodies of the present invention may be “humanized” to prevent the host from mounting an immune response to the antibodies. A “humanized antibody” is one in which the complementarity determining regions (CDRs) and/or other portions of the light and/or heavy variable domain framework are derived from a non-human immunoglobulin, but the remaining portions of the molecule are derived from one or more human immunoglobulins. Humanized antibodies also include antibodies characterized by a humanized heavy chain associated with a donor or acceptor unmodified light chain or a chimeric light chain, or vice versa. The humanization of antibodies may be accomplished by methods known in the art (see, e.g., Mark and Padlan, (1994) “Chapter 4. Humanization of Monoclonal Antibodies”, The Handbook of Experimental Pharmacology Vol. 113, Springer-Verlag, New York). Transgenic animals may be used to express humanized antibodies.

Human antibodies can also be produced using various techniques known in the art, including phage display libraries (Hoogenboom and Winter, (1991) J. Mol Biol. 227:381-8; Marks, et al (1991). J. Mol. Biol. 222:581-97). The techniques of Cole, et al. and Boerner, et al. are also available for the preparation of human monoclonal antibodies (Cole, et al. (1985) Monoclonal Antibodies and Cancer Therapy, Alan R. Liss, p. 77; Boerner, et al (1991). J. Immunol. 147(1):86-95).

Techniques known in the art for the production of single chain antibodies can be adapted to produce single chain antibodies to the immunogenic polypeptides and proteins of the present invention. The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

Bispecific antibodies are monoclonal, preferably human or humanized, antibodies that have binding specificities for at least two different antigens. In the present case, one of the binding specificities is for the identified gene product, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, (1983) Nature 305:537-9). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in Trauneeker, et al. (1991) EMBO J. 10:3655-9.

A particularly preferred aspect of the present invention is an antibody that binds to a polypeptide capable of altering E2F activity and that is used to inhibit the activity of the polypeptide in a patient.

Antibodies as discussed above are also useful in assays for detecting or quantitating levels of a polypeptide capable of altering E2F activity. In one embodiment, these assays provide a clinical diagnosis and assessment of such polypeptides in various disease states and a method for monitoring treatment efficacy.

The present invention provides biologically compatible compositions comprising the polypeptides, polynucleotides, vectors, and antibodies of the invention. A biologically compatible composition is a composition, that may be solid, liquid, gel, or other form, in which the polypeptide, polynucleotides, vector, or antibody of the invention is maintained in an active form, e.g., in a form able to effect a biological activity. For example, a polypeptide of the invention would have an activity that alters E2F activity; a nucleic acid would be able to replicate, translate a message, or hybridize to a complementary nucleic acid; a vector would be able to transfect a target cell; an antibody would bind a polypeptide identified by the present invention. A preferred biologically compatible composition is an aqueous solution that is buffered using, e.g., Tris, phosphate, or HEPES buffer, containing salt ions. Usually the concentration of salt ions will be similar to physiological levels. Biologically compatible solutions may include stabilizing agents and preservatives. In a more preferred embodiment, the biocompatible composition is a pharmaceutically acceptable composition. Such compositions can be formulated for administration by topical, oral, parenteral, intranasal, subcutaneous, and intraocular, routes. Parenteral administration is meant to include intravenous injection, intramuscular injection, intraarterial injection or infusion techniques. The composition may be administered parenterally in dosage unit formulations containing standard, well known non-toxic physiologically acceptable carriers, adjuvants and vehicles as desired.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical compositions for oral use can be prepared by combining active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl-cellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinyl-pyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Preferred sterile injectable preparations can be a solution or suspension in a non-toxic parenterally acceptable solvent or diluent. Examples of pharmaceutically acceptable carriers are saline, buffered saline, isotonic saline (e.g., monosodium or disodium phosphate, sodium, potassium, calcium or magnesium chloride, or mixtures of such salts), Ringer's solution, dextrose, water, sterile water, glycerol, ethanol, and combinations thereof. 1,3-butanediol and sterile fixed oils are conveniently employed as solvents or suspending media. Any bland fixed oil can be employed including synthetic mono- or di-glycerides. Fatty acids such as oleic acid also find use in the preparation of injectables.

The composition medium can also be a hydrogel, which is prepared from any biocompatible or non-cytotoxic homo- or hetero-polymer, such as a hydrophilic polyacrylic acid polymer that can act as a drug absorbing sponge. Certain of them, such as, in particular, those obtained from ethylene and/or propylene oxide are commercially available. A hydrogel can be deposited directly onto the surface of the tissue to be treated, for example during surgical intervention.

Pharmaceutical compositions of the present invention comprise a replication defective recombinant viral vector and the polynucleotide identified by the present invention and a transfection enhancer, such as poloxamer. An example of a poloxamer is Poloxamer 407, which is commercially available (BASF, Parsippany, NJ) and is a non-toxic, biocompatible polyol. A poloxamer impregnated with recombinant viruses may be deposited directly on the surface of the tissue to be treated, for example during a surgical intervention. Poloxamer possesses essentially the same advantages as hydrogel while having a lower viscosity.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended. The formulations to be used for in vivo administration must be sterile. This is readily accomplished by filtration through sterile filtration membranes.

The active ingredients may also be entrapped in microcapsules prepared, for example, by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1980) 16th edition, Osol, A. Ed.

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

The present invention provides methods of treatment, which comprise the administration to a human or other animal of an effective amount of a composition of the invention. A therapeutically effective dose refers to that amount of protein, polynucleotide, peptide, or its antibodies, agonists or antagonists, which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Antibodies according to the invention may be delivered as a bolus only, infused over time or both administered as a bolus and infused over time. Those skilled in the art may employ different formulations for polynucleotides than for proteins. Similarly, delivery of polynucleotides or polypeptides will be specific to particular cells, conditions, locations, etc.

As discussed hereinabove, recombinant viruses may be used to introduce both DNA encoding polypeptides capable of altering E2F activity as well as antisense polynucleotides. Recombinant viruses according to the invention are generally formulated and administered in the form of doses of between about 10⁴ and about 10¹⁴ pfu. In the case of AAVs and adenoviruses, doses of from about 10⁶ to about 10¹¹ pfu are preferably used. The term pfu (“plaque-forming unit”) corresponds to the infective power of a suspension of virions and is determined by infecting an appropriate cell culture and measuring the number of plaques formed. The techniques for determining the pfu titre of a viral solution are well documented in the prior art.

Ribozymes according to the present invention may be administered in a pharmaceutically acceptable carrier. Dosage levels may be adjusted based on the measured therapeutic efficacy.

Methods and Compositions for Lowering Levels of the Activity of Polypeptides Capable of Altering E2F Activity

The methods for decreasing the expression of a polypeptide capable of altering E2F activity and correct those conditions in which polypeptide activity contributes to a disease or disorder associated with an undesirable level of E2F activity include but are not limited to administration of a composition comprising an antisense nucleic acid, administration of a composition comprising an intracellular binding protein such as an antibody, administration of a molecule that inhibits the activity of the polypeptide, for example, a small molecular weight molecule, including administration of a compound that down regulates expression at the level of transcription, translation or post-translation, and administration of a ribozyme which cleaves mRNA encoding the polypeptide.

Methods Utilizing Antisense Nucleic Acids

The present invention, in a particular embodiment, relates to a composition comprising an antisense polynucleotide that is used to down-regulate or block the expression of polypeptides capable of altering E2F activity. In one preferred embodiment, the nucleic acid encodes antisense RNA molecules. In this embodiment, the nucleic acid is operably linked to signals enabling expression of the nucleic acid sequence and is introduced into a cell utilizing, preferably, recombinant vector constructs, which will express the antisense nucleic acid once the vector is introduced into the cell. Examples of suitable vectors include plasmids, adenoviruses, adeno-associated viruses, retroviruses, and herpes viruses. Preferably, the vector is an adenovirus. Most preferably, the vector is a replication defective adenovirus comprising a deletion in the E1 and/or E3 regions of the virus. In a most preferred embodiment, the antisense sequence comprises all or a portion of a polynucleotide complementary to SEQ ID NO: 13.

In another embodiment, the antisense nucleic acid is synthesized and may be chemically modified to resist degradation by intracellular nucleases, as discussed above. Synthetic antisense oligonucleotides can be introduced to a cell using liposomes. Cellular uptake occurs when an antisense oligonucleotide is encapsulated within a liposome. With an effective delivery system, low, non-toxic concentrations of the antisense molecule can be used to inhibit translation of the target mRNA. Moreover, liposomes that are conjugated with cell-specific binding sites direct an antisense oligonucleotide to a particular tissue.

Methods Utilizing Neutralizing Antibodies and Other Binding Proteins

Another aspect of the present invention relates to the down-regulation or blocking of the expression of a polypeptide capable of altering E2F activity by the induced expression of a polynucleotide encoding an intracellular binding protein that is capable of selectively interacting with the polypeptide identified by the present method invention An intracellular binding protein includes any protein capable of selectively interacting, or binding, with the polypeptide in the cell in which it is expressed and neutralizing the function of the polypeptide. Preferably, the intracellular binding protein is a neutralizing antibody or a fragment of a neutralizing antibody. More preferably, the intracellular binding protein is a single chain antibody.

WO 94/02610 discloses preparation of antibodies and identification of the nucleic acid encoding a particular antibody. Using a polypeptide capable of altering E2F activity or a fragment thereof, a specific monoclonal antibody is prepared by techniques known to those skilled in the art. A vector comprising the nucleic acid encoding an intracellular binding protein, or a portion thereof, and capable of expression in a host cell is subsequently prepared for use in the method of this invention.

Alternatively, the activity of a polypeptide capable of altering E2F activity can be blocked by administration of a neutralizing antibody into the circulation. Such a neutralizing antibody can be administered directly as a protein, or it can be expressed from a vector that also codes for a secretory signal.

In another embodiment of the present invention, small molecule compounds inhibit the activity of a polypeptide that alters E2F activity. These low molecular weight compounds interfere with the polypeptide's enzymatic properties or prevent its appropriate recognition by cellular binding sites.

The present invention also involves the use of small molecule compounds to down regulate expression of a polypeptide that is capable of altering E2F activity at the level of transcription, translation or post-translation. Reporter gene systems may be used to identify such inhibitory compounds. These inhibitory compounds may be combined with a pharmaceutically acceptable carrier and administered using conventional methods known in the art.

Methods and Compositions for Increasing Levels of Activity of a Polypeptide Capable of Altering E2F Activity

The methods for increasing the expression or activity of a polypeptide capable of altering E2F activity polypeptide include, but are not limited to, administration of a composition comprising the polypeptide, administration of a composition comprising an expression vector that encodes the polypeptide, administration of a composition comprising a compound that enhances the enzymatic activity of the polypeptide and administration of a compound that increases expression of the gene encoding the polypeptide.

In one embodiment of the present invention, the level of activity is increased through the administration of a composition comprising the polypeptide. This composition may be administered in a convenient manner, such as by the oral, topical, intravenous, intraperitoneal, intramuscular, subcutaneous, intranasal, or intradermal routes. The composition may be administered directly or it may be encapsulated (e.g., in a lipid system, in amino acid microspheres, or in globular dendrimers). The polypeptide may, in some cases, be attached to another polymer.

In another embodiment of the present invention, the intracellular concentration of a polypeptide capable of altering E2F activity is increased through the use of gene therapy, which is through the administration of a composition comprising a nucleic acid that encodes and directs the expression of the polypeptide. In this embodiment, the polypeptide is cloned into an appropriate expression vector. Possible vector systems and promoters are discussed above. The expression vector is transferred into the target tissue using one of the vector delivery systems disclosed herein. This transfer is carried out either ex vivo in a procedure in which the nucleic acid is transferred to cells in the laboratory and the modified cells are then administered to the human or other animal, or in vivo in a procedure in which the nucleic acid is transferred directly to cells within the human or other animal. In preferred embodiments, an adenoviral vector system is used to deliver the expression vector. If desired, a tissue specific promoter is utilized in the expression vector as described above.

Non-viral vectors may be transferred into cells using any of the methods known in the art, including calcium phosphate co-precipitation, lipofection (synthetic anionic and cationic liposomes), receptor-mediated gene delivery, naked DNA injection, electroporation and bio-ballistic or particle acceleration.

Methods Utilizing a Compound that Enhances the Activity of a Polypeptide Capable of Altering E2F Activity

In another embodiment, the activity of the polypeptide is enhanced by agonist molecules that increase the enzymatic activity of the polypeptide or increase its appropriate recognition by cellular binding sites. These enhancer molecules may be introduced by the same methods discussed above for the administration of polypeptides.

In another embodiment, the level of a polypeptide capable of altering E2F activity is increased through the use of small molecular weight compounds, which upregulate expression at the level of transcription, translation, or post-translation. These compounds may be administered by the same methods discussed above for the administration of polypeptides.

Methods Utilizing a Compound that Inhibits the Activity of a Polypeptide Capable of Altering E2F Activity

In another embodiment, the activity of the polypeptide is inhibited by antagonist molecules that decrease the enzymatic activity of the polypeptide or decrease its appropriate recognition by cellular binding sites. These inhibitor molecules may be introduced by the same methods discussed above for the administration of polypeptides.

In another embodiment, the level of a polypeptide capable of altering E2F activity is decreased through the use of small molecular weight compounds, which downregulate expression at the level of transcription, translation, or post-translation. These compounds may be administered by the same methods discussed above for the administration of polypeptides.

The subject invention discloses methods and compositions for the high throughput delivery and expression in a host of sample nucleic acid(s) encoding product(s) of unknown function. Methods are described for infecting a host with the adenoviral vectors that express the product(s) of the sample nucleic acid(s) in the host, identifying an altered phenotype relating to the modulation of E2F activity in the host by the product(s) of the sample nucleic acids, and thereby assigning a function to the product(s) encoded by the sample nucleic acids. The sample nucleic acids can be, for example, synthetic oligonucleotides, DNAs, or cDNAs and can encode, for example, polypeptides, antisense nucleic acids, or GSEs. The methods can be fully automated and performed in a multiwell format to allow for convenient high throughput analysis of sample nucleic acid libraries.

The following examples describe the construction and screening, using an E2F transcriptional assay, of an arrayed adenoviral vector human placenta cDNA. The generation of the placental adenoviral cDNA library used in the present invention, including the construction of the plasmids, adenoviral vectors and the PER.C6 packaging cells are described in U.S. Pat. No. 6,340,595, issued Jan. 22, 2002, in, for example, Examples 1 through 19.

### EXAMPLES

### Example 1

### Library Construction

An arrayed adenoviral human placenta cDNA library is constructed and screened using an E2F reporter assay. Under arrayed adenoviral cDNA library, we mean a collection of adenoviruses (contained in 96-well plates) mediating the expression of various (human) cDNAs, in which every well contains a single virus type. Further details about the concept of arrayed adenoviral libraries are found in WO 99/64582 (Arrayed adenoviral libraries for performing functional Genomics).

Construction of the Primary cDNA Library

Construction of the primary cDNA library is performed as follows. In brief, mRNA emanating from a 12 week old human placenta is used for the (oligo dT-primed) generation of the first strand cDNA using the Superscript II method (Life Technologies). After second strand synthesis, cDNAs are directionally cloned (SalI-NotI) into the pIPspAdapt6 vector (described in WO 99-64582). The cDNA library is then transformed into Escherichia coli (DH10B). 5′ sequencing analysis on 167 clones revealed that 98.8% of the plasmids from the library contained inserts and that 24% of the inserts are full length cDNAs.

Isolation and Storage of Individual cDNA Clones

Parts of the bacteria transformed with the primary cDNA library are plated onto an LB agar growth medium (+100 μg/ml ampicillin) contained in Bio-assay dishes (Life Technologies). These bio-assay dishes are then incubated at 37° C. for 18 hrs. Bacteria are plated at a density of 1500 cfu/plate, thereby allowing recognition and automatic picking of individual colonies by a QPix apparatus (Genetix). This device picked individual bacterial colonies and further inoculated 300 μl of liquid LB growth medium (+100 μg/ml ampicillin) in 96-well plates. Inoculation occurred in such a way that every single well of the 96-well plate is inoculated with bacteria emanating from a single colony. These 96-well plates are incubated for 18 hrs in a rotary shaker (New Brunswick Scientific, Innova, floor model) at 37° C., 300 rpm. After this incubation period, bacterial cultures reach an OD (600 nm) of approximately 4. 100 μl of bacterial cultures are mixed with 100 μl of 50% glycerol using a Multimek robot (Beckman Coulter) and stored at −80° C. These plates are defined as ‘glycerol stock plates’.

Preparation of Plasmid DNA

A second step in the construction of the adenoviral cDNA library is the arrayed purification of DNA of individual plasmids from the primary cDNA library in amounts sufficient for adenovirus generation. For this purpose, a bacterial culture is prepared as follows. The glycerol stock plates are thawed and 3 μl of the bacterial culture is transferred to a 96-well plate filled with 280 μl of liquid LB growth medium (+100 μg/ml ampicillin) using a CybiWell robot (CyBio). These inoculated plates are incubated for 18 hrs in a rotary shaker (37° C., 300 rpm) (New Brunswick Scientific, Innova, floor model). This incubation step yields bacterial cultures with an OD (600) of approximately 8. Centrifugation of the 96-well plates (3 min, 2700 rcf) is performed to pellet the bacteria. All centrifugations of 96-well plates are performed in an Eppendorf microtiterplate centrifuge (type 5810). The supernatant is removed by decanting into a waste container. The lysis of bacterial cells and precipitation of proteins and genomic DNA is performed using the classical alkaline lysis protocol. The (3) buffers for performing alkaline lysis are purchased from Qiagen. In a first step, the bacterial pellet is resuspended into 60 μl of buffer P1. In a second step, 60 μl of buffer P2 is added to the resuspended bacterial cells and a mixing step and 5 min incubation time are applied to achieve complete cell lysis. Finally, 60 μl of buffer P3 is added and a mixing step applied for precipitation of proteins and genomic DNA. The 96-well plates are centrifuged (40 min, 3220 rcf). The supernatant (100 μl) is collected and transferred to new V-bottom 96-well plates containing 80 μl of isopropanol (for precipitation of the plasmid DNA) using a CybiWell robot (CyBio). The plates containing the pellet are discarded. The 96-well plates are centrifuged (45 min, 2700 rcf) and the supernatant discarded by decanting in a waste container. To remove salt traces, the pellet is washed with 100 μl of 70% ethanol and the 96-well plates are centrifuged again (10 min, 2700 rcf). Supernatant is removed again by decanting in a waste container and the DNA pellets are allowed to dry for 1 h in a laminar air flow cabinet. Finally, the DNA is dissolved in 20 μl of sterile TE buffer (1 mM Tris (pH 7.6), 0.1 mM EDTA). Plates containing the dissolved DNA (further defined as ‘DNA plates’) are stored at −20° C. until further use.

DNA Quantification

Before use for transfection of Per.C6/E2A cells, the plasmid DNA preparations contained in 96-well plates are quantified. For this purpose, 5 μl of plasmid DNA is pipetted from the DNA plates and transferred to a 96-well plate containing 100 μl of TE buffer. Then 100 μl of ‘quantification solution’ is added. This solution is prepared by dissolving 2 μl of SybrGreen (Molecular Probes) into 10 ml of TE Buffer. After a mixing step, measurement is performed in a Fluorimeter (Fluostar, BMG) with the following settings: emission: 485 nm; excitation: 520 nm, gain: 35. A standard curve is generated by performing a measurement using different dilutions (in TE buffer) of a standard DNA sample (lambda DNA). By fitting results for the individual DNA samples on this curve, DNA concentration per well is calculated. The mean DNA concentration per well for each ‘DNA plate’ is calculated. On average, a DNA concentration of 20 ng/μl of DNA is obtained.

Transfection of Per.C6/E2A Cells

As mentioned in the description of the primary cDNA library construction, cDNAs produced from the placenta tissue are cloned into the pIPspAdApt6 plasmid. This adapter plasmid contains the 5′ part (bp 1-454 and bp 3511-6093) of the adenovirus serotype 5 genome (in which the E1A gene is deleted and a CMV promoter, multiple cloning site and SV40-derived polyadenylation signal have been inserted). Two other materials needed for the generation of recombinant adenovirus particles are a cosmid and a packaging cell line (see WO99/64582). The cosmid (pWE/Ad.AflII-rITRΔE2A) contains the main part of the adenovirus serotype 5 genome (bp 3534-35953) from which the E2A gene is deleted. The Per.C6/E2A packaging cell line is derived from human embryonic retina cells (HER) transfected with plasmids mediating the expression of the E1 and E2A genes.

In order to obtain viruses, this adapter plasmid is cotransfected into a packaging cell line Per.C6/E2A with the cosmid. Once the adapter and helper plasmids are transfected into the Per.C6/E2A cell line, the complete Ad5 genome is reconstituted by homologous recombination. The helper and adapter plasmids contain homologous sequences (bp 3535-6093), which are a substrate for this recombination event. The E1 and E2A gene products, which are required for adenoviral replication, are provided by the Per.C6/E2A cell line in trans. The adenoviral genes integrated into the genome of the Per.C6/E2A cell line and the reconstituted adenoviral genome share no homologous sequences, which renders the reversion to replication competent adenoviral particles virtually impossible.

The DNA plates that are prepared and quantified as described above, are used for transfection of the Per.C6/E2A cell line. Prior to this transfection, the plasmids contained in these plates are linearized by digestion with the PI-PspI restriction enzyme (New England Biolabs). For this purpose, a certain volume of plasmid DNA (representing 66.7 ng of DNA on average, as calculated from the average DNA concentration of each DNA plate) is pipetted from the DNA plates into a V-bottom 96-well plate containing a restriction mix composed of lx restriction buffer (New England Biolabs: 10 mM Tris-HCl (pH 8.6), 10 mM MgCl₂, 150 mM KCl, 1 mM DTT), 1 00g/ml BSA and 6 units of PI-PspI restriction enzyme (from a stock of 20 U/μl). For each DNA plate, an identical volume of plasmid is used for all wells. Transfer of the DNA samples from the DNA plate to the plate containing the restriction mix and subsequent mixing is performed with a JoBi Well robot (CyBio). The plates containing the restriction mix are put in plastic boxes containing humidified paper towels (to avoid evaporation) and incubated at 65° C. for 4 hrs. The helper plasmid (pWE/Ad.AflII-rITRΔE2A) (which is prepared in batch using the Qiagen Maxi-prep kits) is also linearized with the PacI restriction enzyme (New England Biolabs).

The transfection of the Per.C6/E2A cells with the linearized adapter and helper plasmids is set up as follows. 0.1867 μl of linearized helper plasmid (containing 93 ng of DNA) is mixed with 1.11 μl of serum free 2× DMEM (Life Technologies) to form a helper mix. 0.597 μl of Lipofectamine (Life Technologies) is mixed to 1.11 μl of 2× DMEM to form a lipo mix. In each well of 96-well plates containing the linearized adapter plasmids, 1.3 μl of helper mix and 1.7 μl Lipo mix are pipetted using a CyBi-Well robot (CyBio, equipped with a dropper). The plates are then incubated for approximately 1 hour at room temperature before addition of 28.5 μl of serum-free DMEM. Mixing is performed by pipetting up and down the mix three times (CyBi Well robot). Using the same device, 30 μl of the mix is transferred to 96-well plates containing Per.C6/E2A cells seeded at a density of 2.25×10⁴ cells/well. Cells are seeded into 100 μl of Per.C6/E2A medium (composed of DMEM (Life Technologies) containing 10% FBS (Life Technologies), 50 μg/ml gentamycin and 10 mM MgCl₂), but prior to addition of the 30 μl of the DNA/Lipofectamine mix, the medium is removed from (all wells of) the plates. An incubation time of 3 hours at 39° C., 10% C0₂ is applied. 170 μl of Per.C6/E2A medium is added to the plates and an overnight incubation at 39° C., 10%CO₂ applied. The 96-well plates containing the transfected Per.C6/E2A cells are incubated at 34° C., 10% CO₂ during 3 weeks. This temperature allows the expression of the E2A factor, which is required for adenoviral replication. During this incubation time, viruses are generated and replicated, as revealed by the appearance of CPE (cytopathic effect). The percentage of the wells showing CPE is scored, which allowed the evaluation of the efficiency of virus production. Typically, 55% to 65% of all wells processed show CPE at this stage. The 96-well plates are stored at −80° C. until further propagation of the viruses.

Virus Propagation

The final virus propagation step aims to obtain a higher percentage of wells showing CPE and more homogenous virus titers. Viruses are propagated according to following procedure. The transfection plates stored at −80° C. are thawed at room temperature for about 1 hour. By means of a 96 channel Hydra dispenser (Robbins), 20 μl of the supernatant is transferred onto Per.C6/E2A cells seeded in 96-well plates at a density of 2.25×10⁴ cells/well in 180 μl of DMEM +10%FBS. After handling of a series of 96 viruses, needles of the dispenser are disinfected and sterilised by pipetting up 60 μl of 5% bleach three times. The traces of bleach present in the needles are removed by 3 successive washes with 70 μl of sterile water. Cells are incubated at 34° C., 10% CO₂ during approximately 10 days and the number of wells showing CPE is scored. On average, the number of wells showing CPE increases by 10% as compared to the original scoring after transfection, which represents 65% to 75% of the total number of wells processed. The plates are stored at −80° C. until aliquots are made.

From the 200 μl of crude cell lysate containing the library viruses after the propagation step, 6 aliquots of 25 μl are prepared in 384-well plates using a 96-channel Hydra dispenser. This implied that from 4 96-well plates, 6 identical 384-well aliquot plates are prepared. Disinfection of the needles in between the individual plates is achieved by a triple washing step with 200 μl 5% bleach and a triple washing step with 250 μl sterile water to remove bleach traces. The 384-well aliquot plates are then stored at −80° C. until further use in the assays.

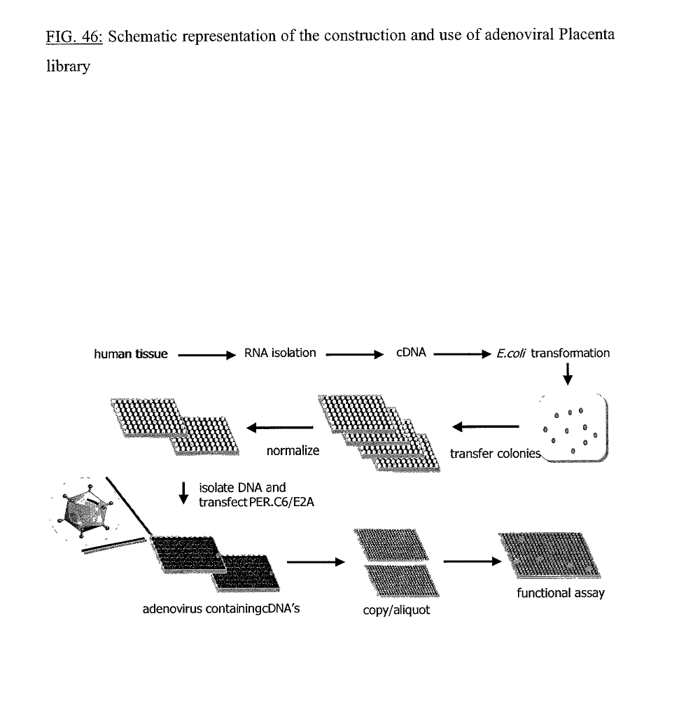

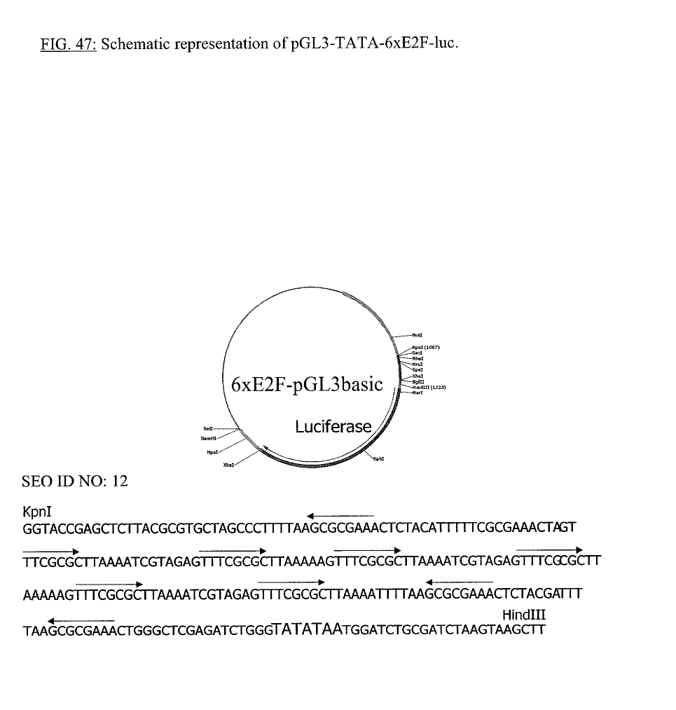

A schematic representation of the library construction is shown in FIG. 46.

### Example 2

### Construction U2OS E2F Reporter Cell Line

Generation of Stable E2F-luciferase reporter in U2OS

Day 1: 4×10 cm dishes with 70% confluent U2OS cells are transfected with the calcium phosphate precipitation technique (van der Eb and Graham, (1980) Methods Enzymol. 65:826-39) according to the following transfection table:

TABLE 1
Transfection table.

| | | |
|---|---|---|
| #1 | #2 | #3 |

| | | | | | | |
|---|---|---|---|---|---|---|
| pBABE-puro | 1 | μg | 1 | μg | 1 | μg |
| 6xE2F-luc | 10 | μg | 10 | μg | 10 | μg |
| CMV-renilla | 1 | μg | 1 | μg | 1 | μg |

| | | | | | |
|---|---|---|---|---|---|
| CMV-E2F1 | — | 0.5 | μg | 2.5 | μg |

Day 2: Plates are washed twice in PBS and fresh medium is added. Cells are cultured in Dulbecco's modified eagle's medium containing 10% fetal calf serum (FBS) supplemented with penicillin/streptomycin.

Day 3: Cells are split 1:5, 1:50, 1:100, 1:200, 1:500.

Day 4: Medium is replaced with medium containing 1 μg/ml puromycin and is refreshed every third day.

Day 22: Medium is removed and the plates are incubated at 37° C. for 4 minutes in PBS. Colonies are picked using a p200 pipette and transferred to a 24-wells plate containing medium with puromycin. 50 colonies from #1, 25 from #2 and 25 from #3 (Table 1) are isolated. Medium is refreshed every second day following day 22.

Day 36: 100 clones grown up from day 22 are split 1:4 and reseeded in 24-wells plates. Medium is changed every second day following this.

Day 42: 48 out of 50 clones from #1 are frozen for storage in liquid nitrogen, 2 are lost under selection.

Day 43: 24 of each #2 and #3 are frozen and stored in liquid nitrogen.

Day 42/43: One well of each clone is split in two and used for first round selection

All clones are tested in 24-well plates for induction of the luciferase reporter by E2F, and repression by p16^{INK4a} and p27^{KIP}. Results are normalized for Renilla expression. From these initial experiments (data not shown), 5 cell lines are chosen that are further tested on 96-well plates.

### Example 3

### Optimization E2F Assay in 96-Well Format

The 5 above mentioned stable U2OS-derived E2F-reporter cell lines (1C5; 1C31; 2C10; 3C1; and 3C20) are tested on 96-well plates. Viruses used are ΔE1/ΔE2A adenoviruses transducing E2F2; E2F3; p16^{INK4a}; p27^{KIP} LacZ; EGFP (all generated from pIPspAdApt plasmids); and empty virus (generated from pIPspAdApt 6).

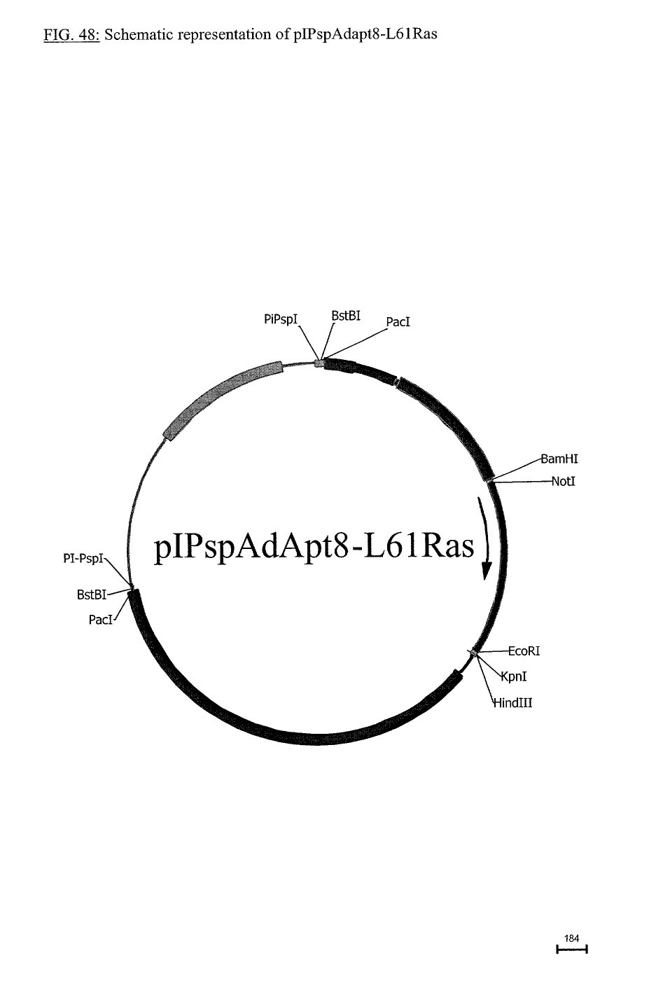

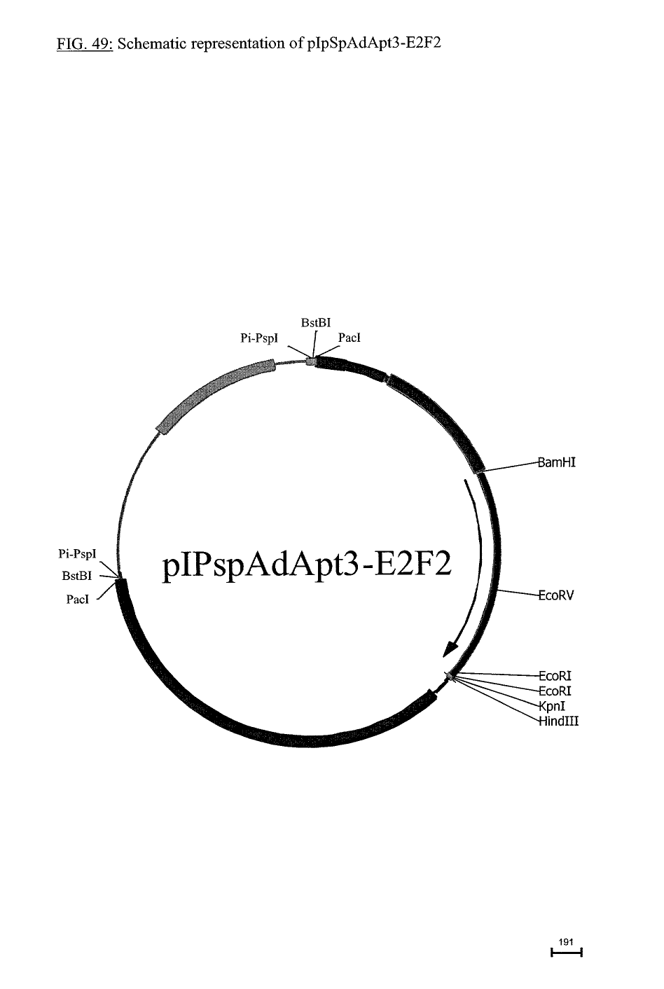

Adenoviral constructs transducing E2F2 and E2F3 are created by digestion of the parental plasmids containing HA-E2F2 and HA-E2F3 cDNAs (Xu, et al. (1995) Proc. Natl. Acad. Sci. USA 92:1357-61) with BamHI and HindIII, isolation of the inserts over an agarose gel, and ligation of the insert fragments in BamHI-HindIII-digested pIPspAdApt 3 (see WO99/64582), to generate pIPspAdApt3-E2F2 and pIPspAdApt3-E2F3, respectively (FIG. 49 and FIG. 50).

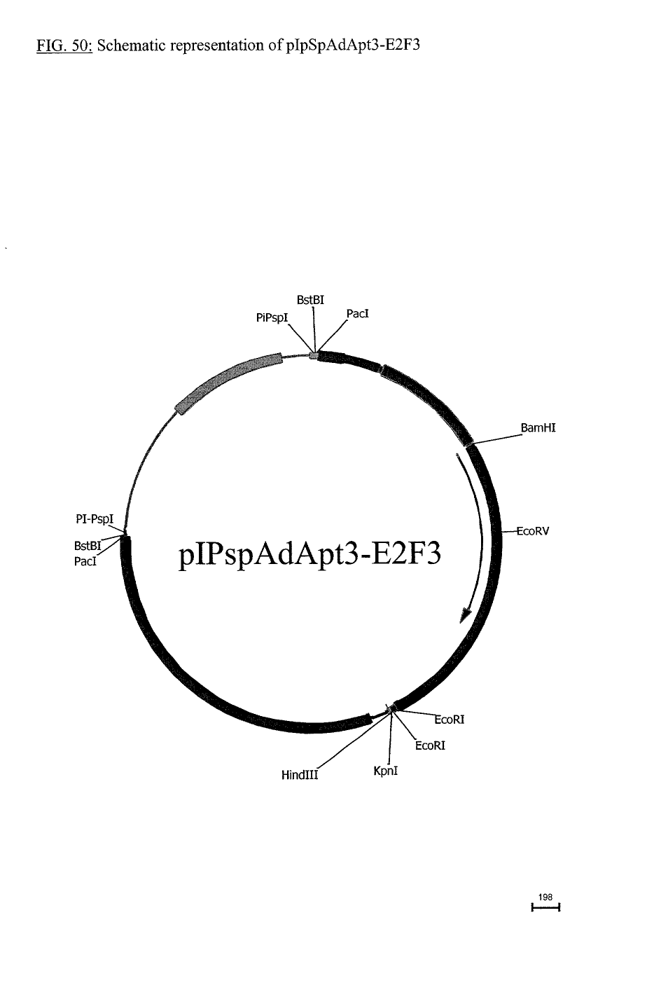

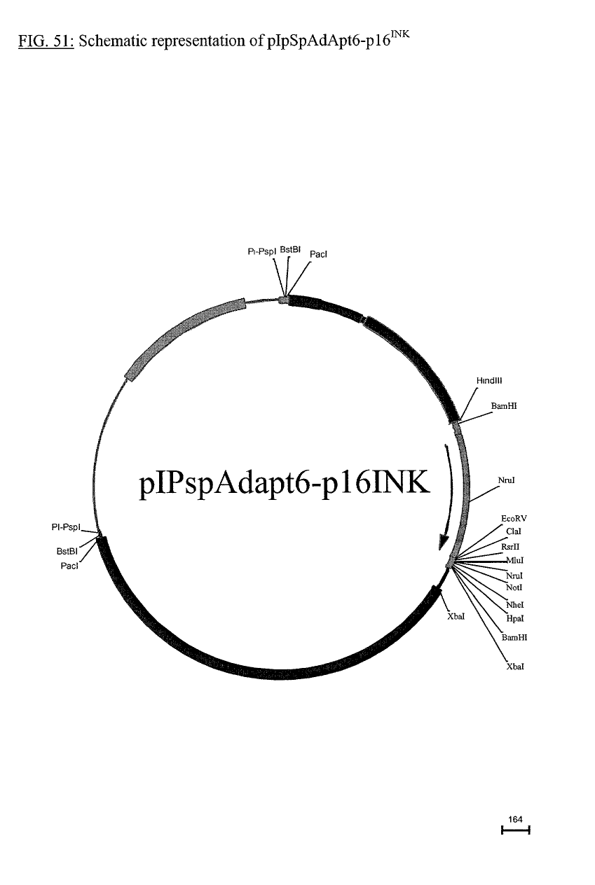

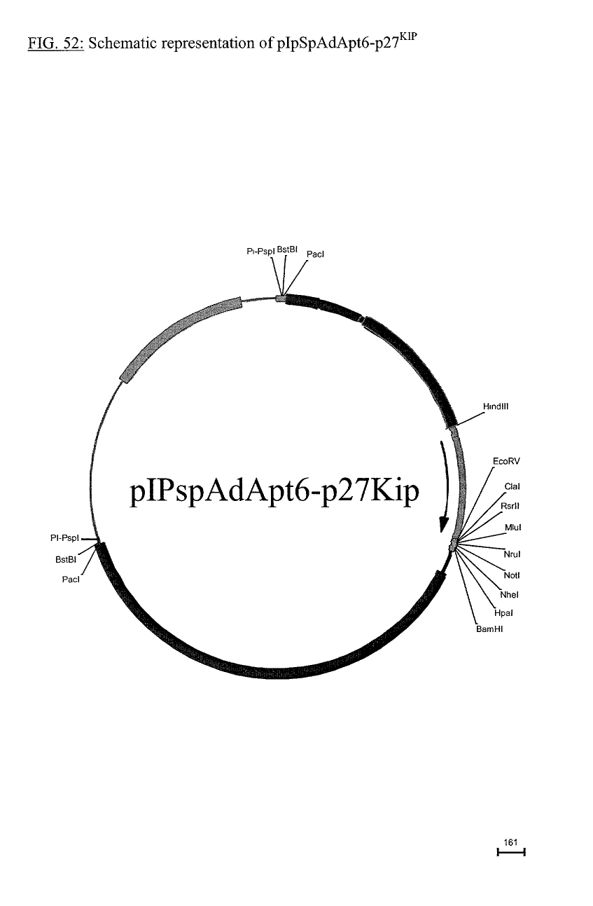

Adenoviral constructs transducing p16^{INK4a} and p27^{KIP} are created by HindIII-XhoI digestion of the parental plasmids containing p16^{INK4a} and p27-HA cDNAs (Beijersbergen, et al. (1995) Genes Dev. 1340-53; Peeper, et al. (1997) Nature 386:177-81) and ligation of the isolated insert fragments in HindIII-SalI-digested pIPspAdApt6 (see WO99/64582), to generate pIPspAdApt6-p16^{INK4a} and pIPspAdApt6-p27^{KIP}, respectively (FIG. 51 and FIG. 52).

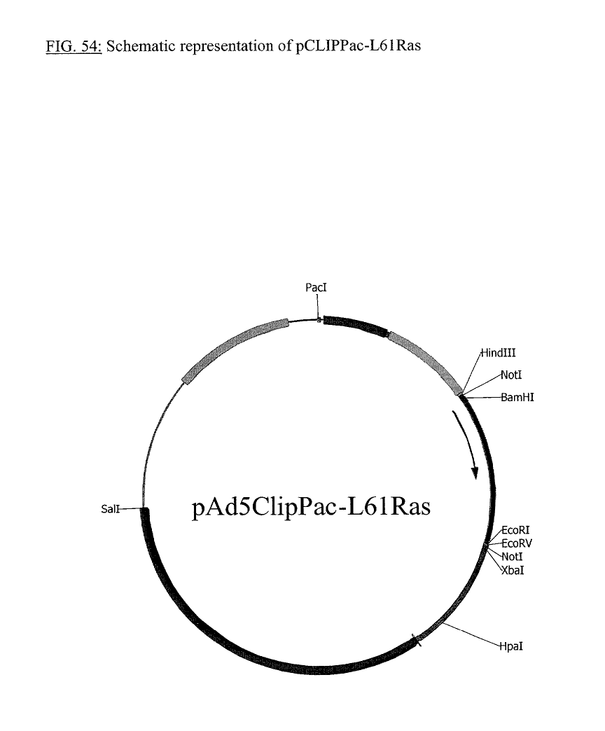

The adenoviral construct transducing L61Ras is created by digestion of the parental construct pMT2SM-L61Ras (Schaap, et al. (1993) J. Biol. Chem. 268:20232-6) with SalI, blunting of the overhang with Klenow polymerase and dNTP's, and digestion with EcoRI. The isolated insert fragment is ligated in pAd5CLIPPac, which is digested with HindIII, blunted with Klenow polymerase and dNTP's, and redigested with EcoRI, resulting in pAd5ClipPac-L61Ras (FIG. 54). The isolated insert fragment is also ligated in HpaI-EcoRI-digested pIPspAdApt 8, leading to pIPspAdApt8-L61Ras (FIG. 48).

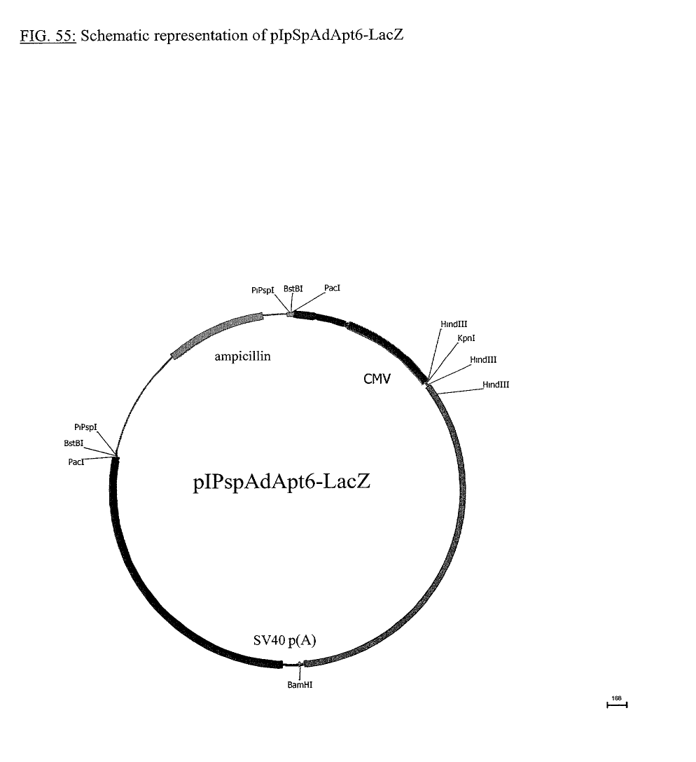

pIPspAdApt6-lacZ (FIG. 55), is constructed by digestion of pIPspAdApt6 with KpnI and BamHI, followed by insertion of the correspondingly digested and purified nls-lacZ gene from pCLIP-lacZ (WO 00/52186).

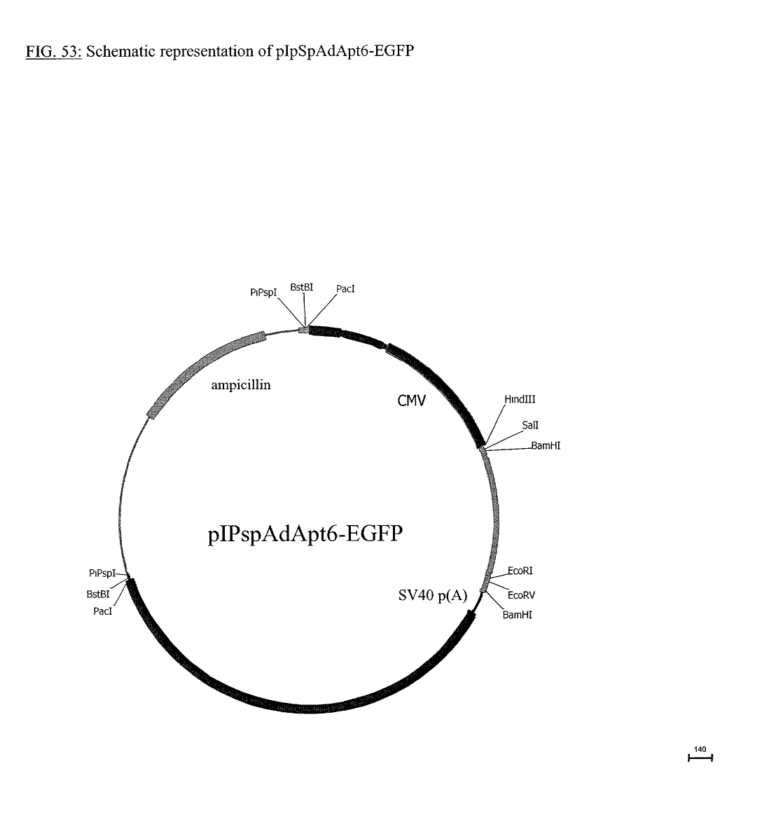

pIPspAdApt6-EGFP, is constructed by releasing the EGFP insert by HindIII-EcoRI digestion from the plasmid pEGFP (Clontech; catalogues number 6077-1), followed by insertion into HindIII-EcoRI-digested pIPspAdApt6 to generate pIPspAdApt6-EGFP (FIG. 53).

ΔE1/ΔE2A adenoviruses are generated from these adapter plasmids by co-transfection of the helper plasmid pWEAd5AflII-rITR.dE2A in PER.C6/E2A packaging cells, as described (WO99/64582).

The 5 E2F-luciferase reporter cell lines are seeded at 5×10³ cells per well in 96-well plates and incubated overnight at 37° C. in a humidified incubator at 10% CO₂ in 100 μl of DMEM supplemented with 10% heat inactivated FBS. The next day, cells are infected with control viruses, transducing p16^{INK4a}, p27^{KIP}, E2F2, E2F3, EGFP, and Empty, at a known MOI of 100 in duplicate.

24 hours after infection, the medium of the 96-well plates is replaced with 100 μl of fresh medium.

72 hours after infection, the medium is removed from the wells. The cells are washed once with Phosphate Buffered Saline and frozen at −20° C. in 100 μl of PBS.

After thawing and resuspension of the cell lysate, 100 μl of Steady-Glo (Promega) is added and incubated for 15 minutes at room temperature. 100 μl of each well of the resulting mixture, is transferred to a Wallac Black&White sample plate and luciferase activity is determined on a Wallac Trilux 1450 microbeta Liquid Scintillation and Luminescence Counter.

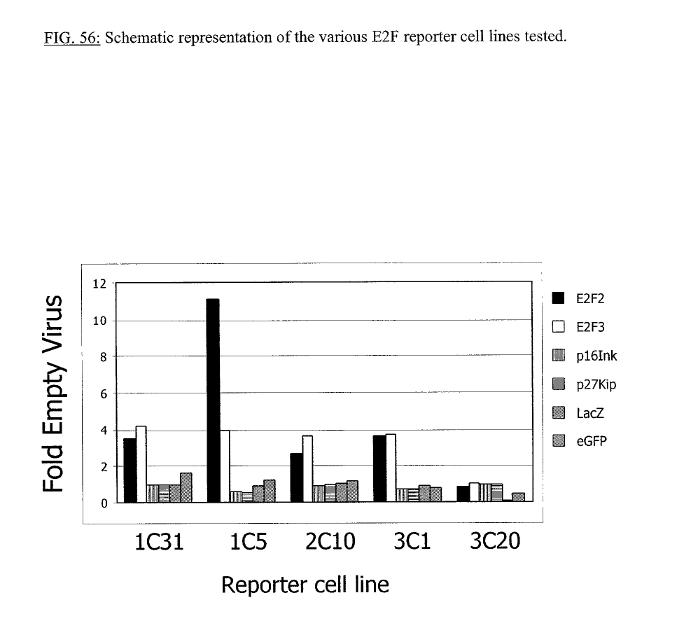

Results are expressed relative to the empty vector control for each cell line (see FIG. 56). From these experiments, it is concluded that cell line IC5 gave the best activation of the luciferase reporter after infection with E2F2- or E2F3-transducing viruses, while repression by p16^{INK4a} or p27^{KIP} could also be scored (see also FIG. 56). Further experiments to optimise the set up of the assay are therefore performed with cell line 1C5.

To determine the optimal MOI for infection, 5×10³ U2OS-1C5 cells are seeded per well in a 96-well plate, using DMEM with 10% heat inactivated FBS and 1 μg/ml puromycin (Clontech) (hereinafter referred to as U2OS medium), and incubated overnight at 37° C. in a humidified incubator at 10% CO₂.

After 24 hours, cells are infected with adenoviruses transducing E2F2, E2F3, p16^{INK4a}, p27^{KIP}, LacZ, EGFP and empty. MOI used are 20, 100 and 500. All experiments are done in triplicate. Infections are allowed for 24 hours after which the medium is replaced with fresh U2OS medium. After a further 24 hours, cells are washed with Phosphate Buffered Saline (PBS) and frozen at −20° C. in 100 μl of PBS.

After thawing and resuspension of the cell lysate, 75 μl of each well is transferred to a fresh plate, 75 μl of Steady-Glo (Promega) is added and incubated for 15 minutes at room temperature. 100 μl of the resulting mixture is transferred to Wallac Black&White sample plates and luciferase activity is determined on a Wallac Trilux 1450 microbeta Liquid Scintillation and Luminescence Counter.

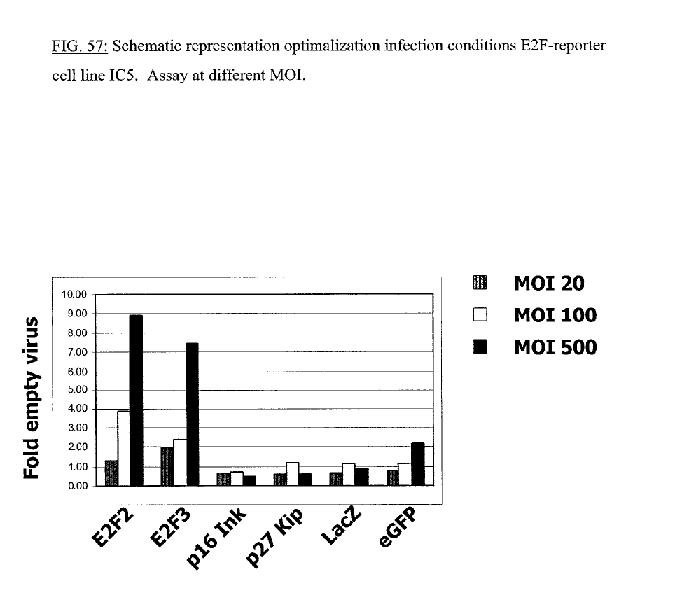

Results are summarized in FIG. 57. Obviously, an MOI of 500 for E2F2 and E2F3 gives the highest induction of the E2F-luciferase reporter. Repression by p16^{INK4a} and p27^{KIP} is more difficult to monitor, but the highest repression is also seen with the highest MOI.

In a further experiment, we analyse whether the length of incubation after infection would influence the outcome of the experiments.

5×10³ U2OS-1C5 cells are seeded per well in a 96-well plate, using U2OS medium, and incubated overnight at 37° C. in a humidified incubator at 10% CO₂. A total of two plates are used.

After 24 hours, cells are infected with adenoviruses transducing E2F2, E2F3, p16^{INK4a}, p27^{KIP}, LacZ, EGFP and empty. MOI used are 100 and 500. All experiments are done in triplicate on the two plates. Infections are allowed for 24 hours after which the medium is replaced with fresh U2OS medium. After a further 24 hours, one of the plates is washed with PBS and frozen at −20° C. in 100 μl of PBS. The remaining plate is washed and frozen 24 hours later.

After thawing and resuspension of the cell lysate, 75 μl of each well is transferred to a fresh plate, 75 μl of Steady-Glo (Promega) is added and incubated for 15 minutes at room temperature. 100 μl of the resulting mixture is transferred to Wallac Black&White sample plates and luciferase activity is determined on a Wallac Trilux 1450 microbeta Liquid Scintillation and Luminescence Counter.

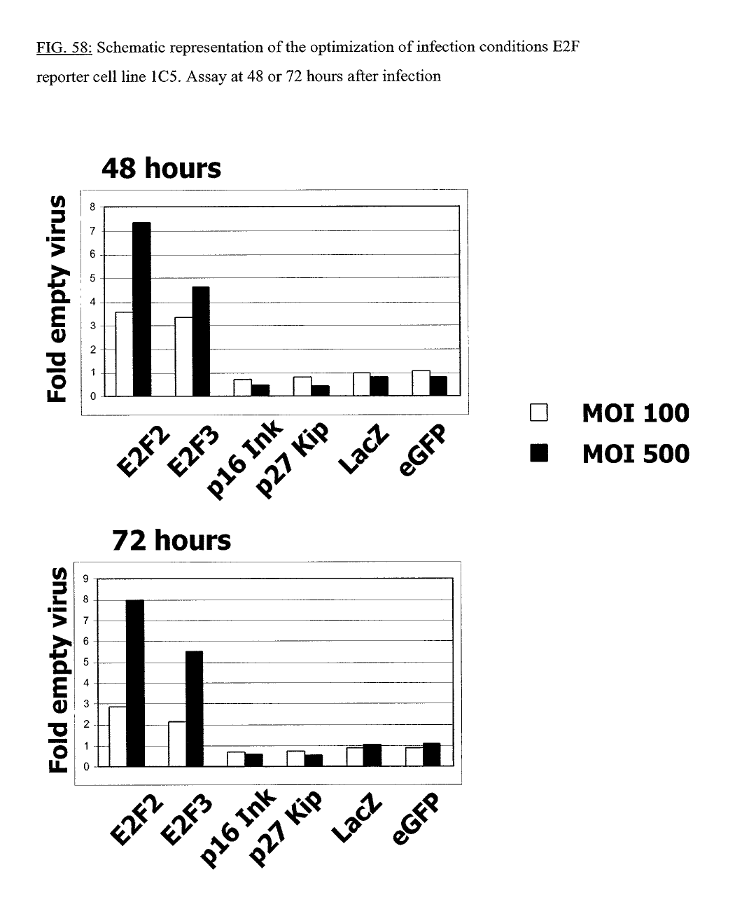

Results are summarized in FIG. 58. As can be seen in these figures, activation of the E2F-reporter by E2F2 and E2F3 is comparable between 48 hours and 72 hours infection time. However, repression by p16^{INK4a} and p27^{KIP} is more pronounced after 48 hours compared to 72 hours. It therefore is concluded that the optimal length of infection is 48 hours.

In an attempt to make repression of the E2F-luciferase reporter by p16^{INK4a} and p27^{KIP} more pronounced, we performed co-infection experiments with different MOI of E2F2 to enhance the basic expression of the reporter. In the same experiment, the effect of reducing the amount of FBS from 10% to 2% is examined.

For this, 5×10³ U2OS-1C5 cells are seeded per well in a 96-well plate, using U2OS medium, and incubated overnight at 37° C. in a humidified incubator at 10% CO₂. A total of 3 plates are seeded.

The next day, plate 1 is infected with adenoviruses transducing E2F3, p16^{INK4a}, p27^{KIP}, LacZ, EGFP, empty, and pClip-L61Ras. MOI used are 100 and 500, each in triplicate, using half of the plate. Infections are duplicated on the second half of the plate. The same layout is used to infect plate two and three. However, all wells from plate 2 are co-infected with MOI 20 of adenovirus transducing E2F2, while all wells of plate 3 are co-infected with MOI 100 of adenovirus transducing E2F2.

Infections are allowed for 24 hours after the medium on the first half of the plates is replaced with fresh U2OS medium, while on the second half of the plates, it is replaced with U2OS-medium containing 2% FBS. After a further 24 hours, all plates is washed with PBS and frozen at −20° C. in 100 μl of PBS.

After thawing and resuspension of the cell lysate, 75 μl of each well is transferred to a fresh plate, 75 μl of Steady-Glo (Promega) is added and incubated for 15 minutes at room temperature. 100 μl of the resulting mixture is transferred to Wallac Black&White sample plates and luciferase activity is determined on a Wallac Trilux 1450 microbeta Liquid Scintillation and Luminescence Counter.

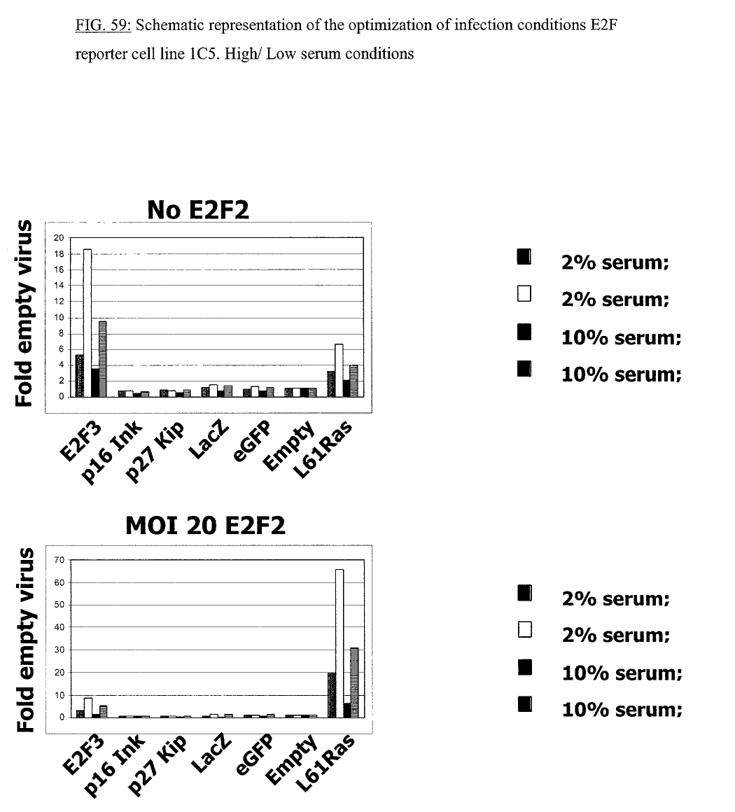

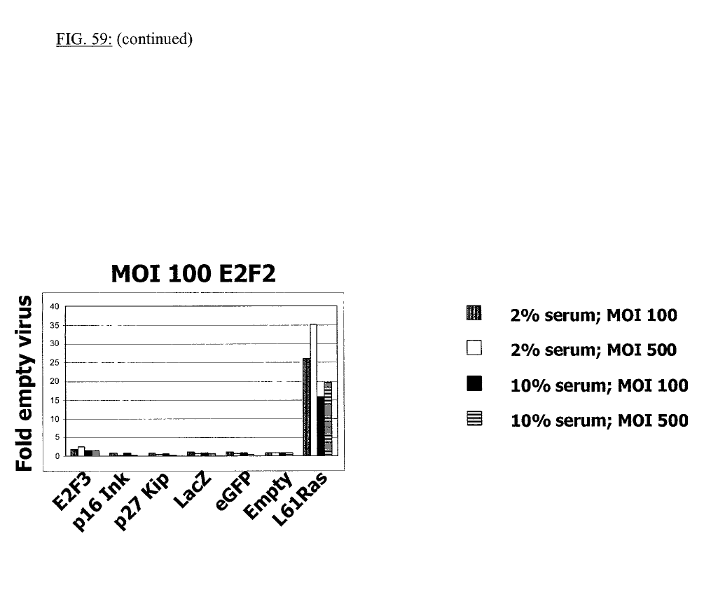

Results are summarized in FIG. 59. Induction of the E2F-luciferase reporter by E2F3 and L61 Ras is MOI-dependent, with more induction at higher MOI, and is more pronounced at 2% FBS of than at 10% FBS. Repression by p16^{INK4a} and p27^{KIP} does not differ significantly between the two growth conditions.

When co-infected with MOI 20 of E2F2, the basic signal is higher than without co-infection and the fold induction over empty virus is less for E2F3. This effect is even higher when co-infecting with MOI 100 of E2F2.

L61Ras, however, seems to co-operate with E2F2 in that the fold induction over empty virus is dramatically increases when co-infected with MOI 20 or 100 of E2F2. The induction by L61Ras, co-infected with MOI 20 or 100 of E2F2, is even 5 fold higher than the induction by E2F3 after co-infection with MOI 20 or 100 of E2F2, while induction of L61Ras in the absence of E2F2 is less than that of E2F3. This suggests some synergism between the Ras- and E2F-pathways.

Co-infection with E2F2 did not clearly result in a more pronounced repression of the E2F-luciferase by p16^{INK4a} and p27^{KIP}.

Therefore, since the effects of serum reduction and co-infection of E2F2 did not result in more pronounced reduction of the E2F-luciferase reporter by p16^{4a} and p27^{KIP}, these conditions are not used for the screenings.

### Example 4

### E2F Screen with 1500 Adenoviruses in 96-Well Format

To determine the feasibility of the E2F-reporter assay, a random 1440 viruses of the placenta library are picked and used to infect the U2OS 1C5 reporter cell line.

For this, U2OS 1C5 reporter cells are seeded at a density of 5×10³ cells per well in a 96-well plate and incubated overnight at 37° C. in a humidified incubator at 10% CO₂ in 100 μl of DMEM supplemented with 10% heat inactivated FBS.

The next day, cells are infected with 10 μl of crude lysate of 15 cherry picked propagated virus plates of the adenoviral placenta library in a total volume of 20 μl. The assumed titre of this library is 5×10⁸ virus particles per ml, resulting in a MOI of 1000.

Control viruses, transducing p16^{INK4a}, p27^{KIP}, E2F2, E2F3, EGFP, and Empty, are included at known MOI of 10, 100, and 1000 in duplicate. Two independent virus preparations are used for p16^{INK4a}, p27^{KIP}, E2F2, and E2F3. 24 hours after infection, the medium of the 96-well plates is replaced with 100 μl of fresh medium.

48 hours after infection, the medium is removed from the wells and the cells are washed once with Phosphate Buffered Saline and frozen at −20° C. in 100 μl of PBS.

After thawing and resuspension of the cell lysate, 50 μl of each well is transferred to a Wallac Black&White sample plate and 50 μl of Steady-Glo (Promega) is added and incubated for 15 minutes at room temperature. Luciferase activity is determined on a Wallac Trilux 1450 microbeta Liquid Scintillation and Luminescence Counter.

The whole experiment is performed twice. Empty virus gives mean luciferase readings of 17.3 and 15.6 relative light units, respectively, in the two experiments, with standard deviations of 2.6, and 2.2, respectively.

At MOI 10, E2F2 and E2F3 expression causes a 1.5 to 3.1 increase of the luciferase signal, compared to empty virus control. At MOI 100, E2F2 and E2F3 expression causes a 2.3 to 8.3 fold induction of the luciferase signal, compared to empty virus control. At MOI 1000, induction by E2F2 and E2F3 is between 7.1 and 10.9 fold empty virus.

Repression by p16^{INK4a} and p27^{KIP} is more difficult to monitor. In general, the highest MOI results in the highest repression. At MOI 1000, the mean repression by p16^{INK4a} is 0.7 fold empty virus, while p27^{KIP} expression results in a 0.5 fold decrease of the signal of empty virus.

The mean signal of the library is 17.3 and 15.9, respectively, for the two experiments, with standard deviations of 10.5 and 21.6, respectively.

Individual wells are selected that result in both experiments luciferase readings higher than the mean of empty virus plus 4 times the standard deviation, which values are 27.6 and 24.5, respectively.

Individual wells are also selected that gave in both experiments luciferase readings lower than the mean of empty virus minus 4 times the standard deviation, which values are 7.0 and 6.7, respectively.

All potential hits are subjected to a second round of screening (Example 5).

### Example 5

### Rescreen of Hits from 1500 Screen

To propagate the viruses used in the E2F assay, 2.25×10⁴ Per.C6/E2A cells are seeded in 200 μl of DMEM containing 10% non-heat inactivated FBS into each well of a 96-well plate and incubated overnight at 39° C. in a humidified incubator at 10% CO₂. Subsequently, 10 μl of crude lysate, containing the viruses from the placenta library, is added and incubation is proceeded at 34° C. in a humidified incubator at 10% CO₂ for 8 days, after which the plates are frozen at −20° C.

To rescreen the potential hits from the first round, U2OS 1C5 reporter cells are seeded in 96-well plates at a density of 5×10³ cells per well using 100 μl of DMEM supplemented with 10% heat inactivated FBS.

The next day, cells are infected in triplicate using an MOI of 100 and 500 and a total infection volume of 20 μl.

Infections are done with the potential hits as identified in the first round of screening (see example 4) and randomly picked viruses from the same plates as control. We assume a titer of 5×10⁹ virus particles (vp) per ml for the propagated viruses from the library. Known titers are used for the control viruses transducing E2F2, E2F3, p16^{INK4a}, p27^{KIP}, LacZ, EGFP and empty. Viruses transducing E2F2, p16^{INK4a} and empty, are included on all 96-well plates.

24 hours after infection, the medium of the 96-well plates is replaced with 100 μl of fresh medium.

48 hours after infection, the medium is removed from the wells and the cells are washed once with Phosphate Buffered Saline supplemented with 1 mM Ca²⁺ and 1 mM Mg²⁺ (PBS⁺⁺), and frozen away at −20° C. in 100 μl of PBS⁺⁺.

After thawing and resuspension of the cell lysate, 75 μl of each well is transferred to a fresh plate, 75 μl of Steady-Glo (Promega) is added and incubated for 15 minutes at room temperature. 100 μl of the resulting mixture is transferred to a Wallac Black&White sample plate and luciferase activity is determined on a Wallac Trilux 1450 microbeta Liquid Scintillation and Luminescence Counter. Results are calculated as fold activation compared to empty virus.

### Example 6

### Validation Hits from Rescreen 1500

To analyse whether the activation or repression of the luciferase signal after infection of the potential hits in the E2F-reporter cell line U2OS 1C5 (see example 5), is mediated through the E2F-binding sites in the promoter of the reporter, and not through plasmid or genomic sequences flanking the integrated reporter construct, the E2F-luciferase reporter construct and a control reporter construct are transiently transfected in wildtype U2OS cells. Particle titers of these viruses are determined by real-time PCR, as described (Ma, et al. (2001) J. Virol. Methods 93:181-8).

For the transient reporter assay, 3×10⁵ U2OS cells are seeded in each well of a 6-well plate in 2 ml of DMEM +10% heat inactivated Foetal Bovine Serum (U2OS-medium).

The next day, medium is replaced with 1.65 ml of fresh U2OS medium. 2 hours later, individual wells of the 6-well plate are transfected with either the E2F-luciferase reporter construct, or the pGL3-basic control reporter construct. Transfection is performed using the Calcium Phosphate Transfection System according to the manufacturer's protocol (Life Technologies). However, all volumes are adjusted (divided by 6.05), since the protocol is described for a 100 mm tissue culture dish instead of a 6-well dish. The total amount of DNA is 3.3 microgram per well, and identical amounts of reporter DNA and carrier DNA are used. The precipitate is left for 24 hours on the wells.

After 24 hours, cells harvested with Trypsine/EDTA (Life Technologies) and collected in U2OS medium according to standard procedures. 5×10³ transfected U2OS cells are seeded per well in a 96-well plate in 100 μl of U2OS-medium and incubated overnight at 37° C. in a humidified incubator at 10% CO₂.

The next day, viruses encoding potential hits (see above) and control viruses transducing E2F2, p16^{INK4a}, p27^{KIP}, EGFP, LacZ, and Empty, are used to infect U2OS cells transiently transfected with the E2F-luciferase reporter construct, or the pGL3-basic control reporter construct. Cells are infected with the viruses at MOI of 100 and 500. 6-wells of a 96-well plate are used for each MOI for all viruses. Cells are incubated further for 48 hours at 3 7° C. in a humidified incubator at 10% CO₂.

48 hours after infection, the medium is removed from the wells and the cells are washed once with PBS and frozen away at −20° C. in 100 μl of PBS.

After thawing and resuspension of the cell lysate, 50 μl of each well is transferred to a Wallac Black&White sample plate and 50 μl of Steady-Glo (Promega) is added and incubated for 15 minutes at room temperature. Luciferase activity is determined on a Wallac Trilux 1450 microbeta Liquid Scintillation and Luminescence Counter.

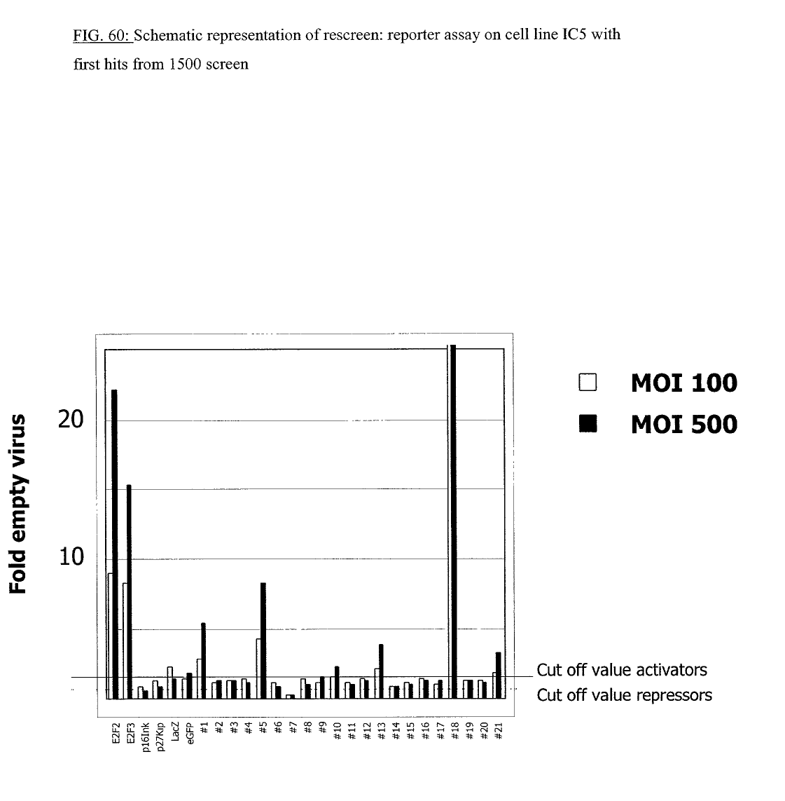

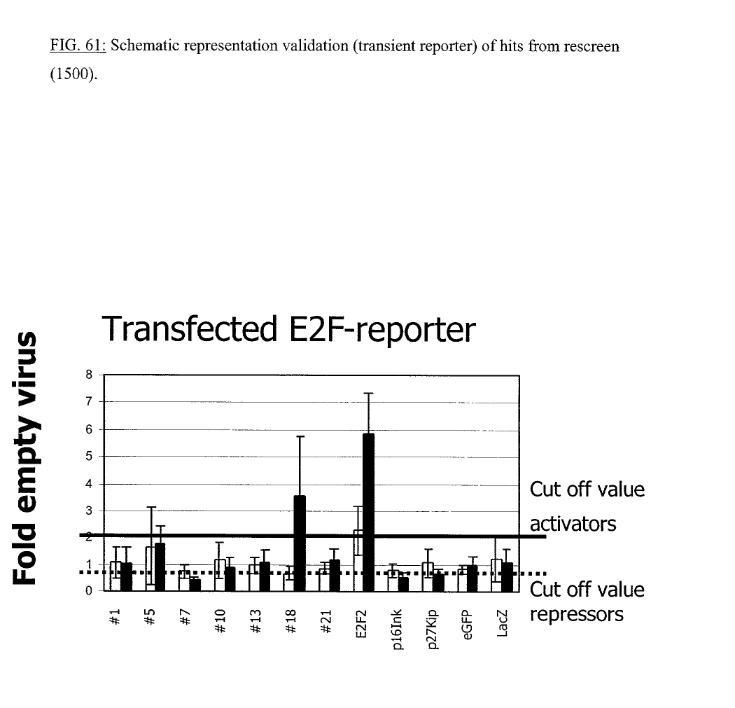

Results are presented relative to empty virus control in FIG. 61. Neither of the potential hits, nor the control viruses, modulated expression of the transfected pGL3-basic control reporter construct (data not shown).

### Example 7

### E2F Screen with 11,000 Viruses in 384-Well Format

Preparation of the Control Plates

Control plates are prepared that contain different control pIPspAdApt viruses transducing the following transgenes: E2F2, E2F3, p16^{INK4a}, p27^{KIP}, GFP or the empty virus (defined as the virus with empty MCS) or no virus at all. These viruses are propagated according to the protocol applied for the Phenoselect library. Day 0, TC treated 96-well plates are seeded with Per.C6/E2A cells at a density of 2.25×10⁴ cells per well in 200 μl medium. Day 1, 48 wells per plate are infected with 20 μl of one type of control virus emanating from a larger batch preparation. After 7 days, full CPE is obtained. The plates are subjected to one freeze-thaw cycle and aliquots are made of the crude virus lysate in 96-well V-bottom plates as follows. The 8 wells of every column are filled with 25 μl of one type of control virus (See FIG. 62).

Column 1: E2F2 virus. Column 2: 1/10 dilution of the E2F2 virus. Column 3: E2F3 virus. Column 4: 1/10 dilution of the E2F3 virus. Column 5: p16^{INK4a}. Column 6: 1/10 dilution of the p16^{INK4a} virus. Column 7: p27^{KIP} virus. Column 8: 1/10 dilution of the p27% virus. Column 9: Empty virus. Column 10: 1/10 dilution of the empty virus. Column 11: GFP virus. Column 12: Medium+10% FBS.

The aliquots are sealed with a seal (Nunc Cat No 236366) and stored at −80° C. until use.

The control plates are tested according to the screening protocol. 8 μl of virus crude lysate is pipetted from a control plate using a 96 channel Hydra dispenser (Robbins Scientific) and 1 μl is dispensed in positions A1, A2, B1 and B2 of a white 384-well plate (Greiner) in which U2OS 1C5 reporter cells are seeded at a density of 1250 cells/well (20 μl medium per well). 48 hrs post-infection, 15 μl of Luciferase substrate (Promega Steady Glow) is added to the wells, the plates are sealed and put on a rotary shaker for 30 min. Readout is then performed in a luminometer (Lumicount, Packard, Gain 150, PMT voltage 1100V). Results are shown in FIG. 62. For the undiluted virus controls, E2F expression causes a 5.8-fold (E2F2) or 4.5-fold (E2F3) rise of the signal as compared to the empty virus infected wells. A 4-fold or 5-fold reduction of the signal is seen when expressing p16^{INK4a} or p27^{KIP}, respectively. A 10-fold dilution of the control viruses results in a 8.8-fold and 3.7 fold activation of the signal as compared to the wells infected with the empty virus for the E2F2 and E2F3 viruses, respectively and zero or a 2-fold reduction of the signal as compared to the empty virus infected wells for p16^{INK4a} and p27^{KIP} respectively. This experiment confirms the quality of the produced control plates and yielded the trends observed previously.

Protocol for Screening of the PhenoSelect Library

U2OS reporter cells 1C5 are cultured in DMEM containing 10% of heath inactivated FBS and 1 μg/ml puromycin. Performing the assay, U2OS cell cultures are strictly kept subconfluent.

Day -3, 5 T175 flasks are seeded with U2OS reporter cells C15 at a density of 1.5×10⁶ cells per flask.

Day 0, T175 flasks seeded day -3 are treated with trypsin/EDTA (2 ml of trypsin/EDTA mix/flask) to detach cells. Cells (resuspended in 10 ml culture medium/T175 culture flask) are counted. Cells are then resuspended in culture medium at a density of 6.25×10⁴ cells/ml for further seeding. White tissue culture treated 384-well plates are seeded at a density of 1.25×10⁴ cells per well, 20 μl per well, using a multidrop (Labsystems).

Day 1: Approximately 18 hours after seeding of the reporter cells, reporter cells are infected with the library viruses as follows.

The virus library aliquot plates (384-well format) to be processed (10 plates per day) are put in a laminar airflow cabinet for 1 hour for thawing. Plates are put at 4° C. until further processing.

For every well of the 384-virus library aliquot plate, 1 μl of virus crude lysate is transferred to three wells (coordinates A1, A2 and B1) of the white 384-well plate containing the seeded reporter cells. This is done using a Hydra 96 dispenser (110 μl) (Robbins Scientific). The pipettor is programmed to fill its syringes with 10 μl of virus crude lysate and to dispense 1 μl at positions A1, A2 and B1 in the plate containing the reporter cells. After this action, syringes are emptied in the original virus library aliquot plate. Before processing of the following virus library aliquot plate, syringes are cleaned by performing 3 washing steps with 20 μl of 5% bleach. The syringes are then rinsed 3 times with 25 μl of sterile deionized water.

After processing of all virus library aliquot plates, the control viruses are added to the plates as follows: for every well of the 96-well control plate, one μl of virus crude lysate is transferred to 1 well, B2 quadrant, on 8 to 10 384-well plates containing the reporter cells infected with the library viruses. (This position is left uninfected during infection of the reporter cells with the library viruses.) Addition of the control viruses is also performed using the Hydra dispenser.

Approximately 48 hours after infection, readout of reporter activation is performed. The luciferase substrate (Steady Glow, Promega) is freshly prepared according to the protocol of the manufacturer. 15 μl of luciferase substrate is added to the wells using the Hydra dispenser. This operation is performed in a laminar airflow cabinet and under subdued light conditions. The dispenser is programmed to fill its syringes with 70 μl of substrate and to sequentially dispense 15 μl to the A1, A2, B 1 and B2 quadrants. The syringes are then refilled for processing the next plate without intermediate washing step. After addition of the substrate, the plates are sealed (Nunc cat N° 236366) and put on a rotary shaker for 30 min. Plates are then sequentially inserted into a luminometer (Lumicount, Packard) for readout. The apparatus is used with the following settings: Gain 150, PMT voltage 1100 V, 0.3 sec reading time. Time in between substrate addition and readout is not allowed to exceed 1 hour. Data are stored in Excel sheet format (Microsoft).

The screening is performed in 4 series of 10×384-well virus aliquot plates, which represents 15360 wells. As the virus production efficiency for the Phenoselect library reached on average 70% of the total amount of wells, this represents approximately 10750 viruses.

Data Analysis.

The data obtained from the luminometer are analysed as follows.

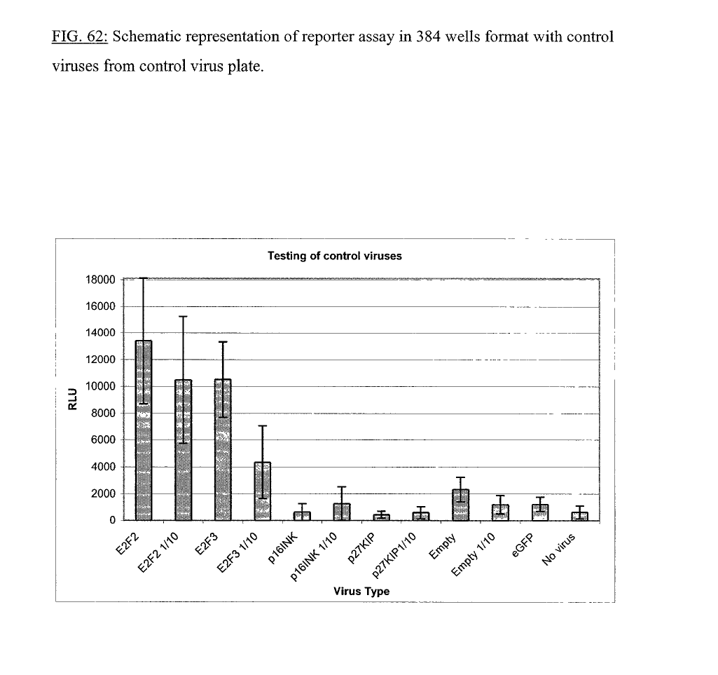

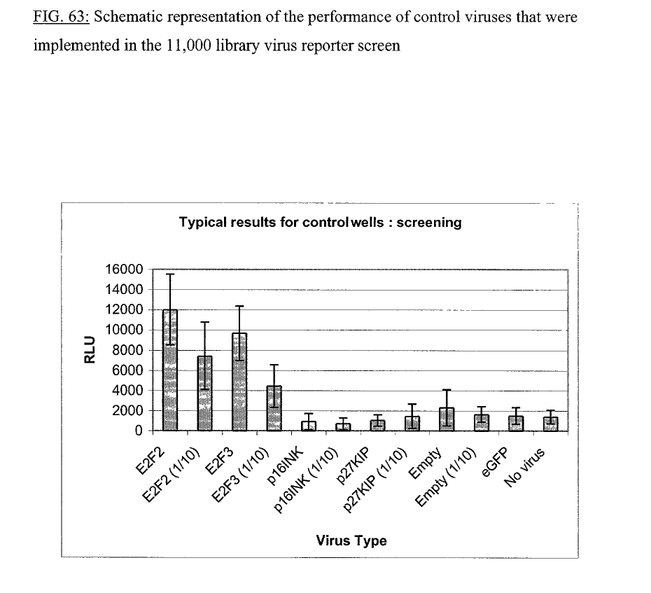

In first instance, the control data inserted in 96 positions of the B2 quadrant in 8 to 10 assay plates per screen are extracted and compiled. Background signal levels associated with the Empty virus and the standard deviation on this measurement are determined. The results obtained for the wells infected with the various control viruses are analysed in order to evaluate the quality of the screening. A typical result for the wells infected with the control viruses during one out of the 4 runs of the screening is shown in FIG. 63. As 8 wells of the control plate contained the same virus, and as reporter cells in 8 to 10 screening plates are infected with the control viruses, each control virus is tested at least 64 times per run. The mean of the 3 values obtained for every individual library virus is calculated. All mean values are sorted. Viruses causing an increase of the signal are considered as hits provided these mediated a signal superior to the cut off value. The cut off value for samples identified as E2F activators is defined as being the mean plus three times the standard deviation of the signal obtained for the wells in which cells are infected with the empty virus. Library viruses that mediated a lower signal as the empty virus-infected wells are considered as hits provided these mediated a signal of at least half of the signal of the 8 neighbor library viruses.

### Example 8

### Rescreen of Hits from 11,000 Screen

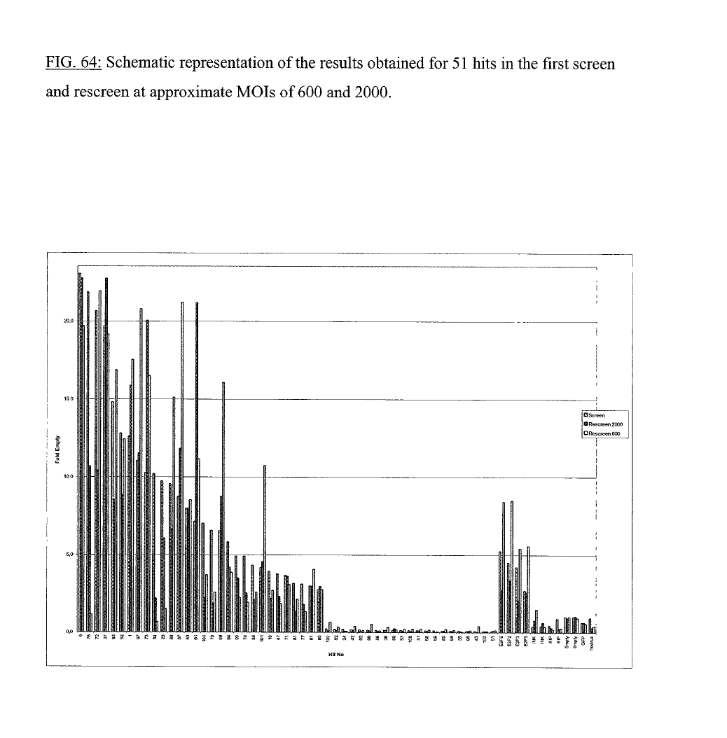

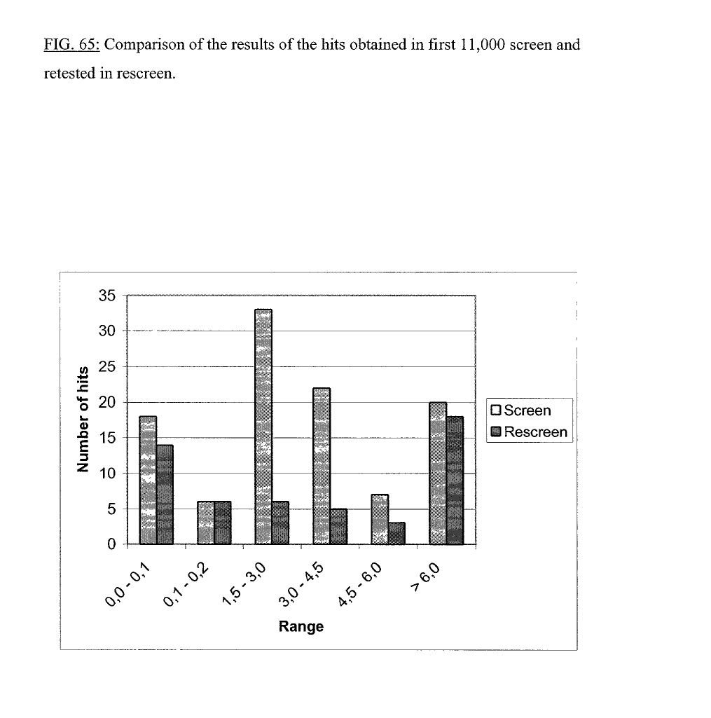

For the viruses scored as hit, two μl of virus crude lysate is recovered from the well of the original 384-well aliquot plates that are used for performing the screening. These aliquot plates are stored at −80° C. and thawed for a second time for removal of this 2 μl aliquot. The viruses of the hits are propagated by using the 2 μl aliquots of crude virus to infect 2.25×10⁴ Per.C6/E2A cells seeded in 96-well plates (200 μl of DMEM+10% FBS). After appearance of complete CPE, these 96-well plates undergo a single freeze-thaw cycle. Four aliquots of 40 μl (stored in V-bottom 96-well plates) are prepared from the 200 μl of supernatant of the infected Per.C6/E2A cells. These aliquots are used for performing the rescreen. The aim of the rescreen is to test the repropagated hit viruses using the stable reporter cell line 1C5 at various MOIs. This rescreen is performed applying the same protocol as the one used for the primary screen (see example 7). Briefly, 1 μl of the undiluted virus crude lysate aliquots (emanating from the repropagation step) and 1 μl from a 3-fold dilution of these aliquots are used to infect the 1C5 U2OS reporter cell line seeded in 384-well plates. (This corresponds to MOI of approximately 2000 and 600, respectively). Two days after infection, luciferase substrate is added and readout is performed. Results of the rescreen are compared to the results of the original screening (FIG. 64; Remark: for clarity of the graph, the value indicated for hit 9 at MOI 600 corresponds to one fourth of the real value and the value indicated for hit 27 at MOI 600 corresponds to one third of the real value.) The cut off value for samples identified as E2F activators is defined as being the mean plus three times the standard deviation of the signal obtained for the wells in which cells are infected with the empty virus. The viruses mediating a signal lower as the non-infected wells (indicated as “No virus”) are scored as repressors. Applying these cut off values, 27 of the hits are confirmed as activators and 21 hits are confirmed as repressors for the higher MOI. At the lower MOI, 22 hits are confirmed as activators and 15 as repressors. The distribution of the 106 hits obtained in the original screening is represented in FIG. 65. Two ranges are defined for the repressors (One fifth to one tenth or less as one tenth of the empty virus signal) and 4 ranges for the activators (1.5 to 3 fold, 3 to 4.5 fold, 4.5 to 6 fold or more as 6 fold the empty virus signal). In the same graph, the number of hits within the different ranges that are confirmed during the rescreen (at the approximate MOI of 2000) are indicated. From these data, we can conclude that most repressors could be confirmed in the rescreen. For what concerns the activators, the strongest hits (more as 6 fold activation) are generally confirmed, the moderate activators (between 4.5 and 6 fold empty virus) are confirmed in 50% of the cases and the weak activators (less as 4.5 fold empty virus) are generally not confirmed.

### Example 9

### Validation Hits from Rescreen 11,000

To propagate the potential hits of the E2F assay, 2.25×10⁴ Per.C6/E2A cells are seeded in 200 μl of DMEM containing 10% non-heat inactivated FCS into each well of a 96-well plate and incubated overnight at 39° C. in a humidified incubator at 10% CO₂. Subsequently, 5 μl of crude lysate, containing the viruses from the placenta library, is added to two of the wells and incubation is proceeded at 34° C. in a humidified incubator at 10% CO₂ for 12 days, after which the plates are frozen at −20° C.

Particle titers of these viruses are determined by real-time PCR, as described (Ma, et al. (2001) J. Virol. Methods 93:181-8).

For the transient reporter assay, 3×10⁵ U2OS cells are seeded in each well of a 6-well plate in 2 milliliter of DMEM +10% heat inactivated Foetal Calf Serum (U2OS-medium).

The next day, medium is replaced with 1.65 ml of fresh U2OS medium. 2 hours later, individual wells of the 6-well plate are transfected with either the E2F-luciferase reporter construct, or the pGL3-basic control reporter construct (Promega), or the pGL3-promoter control reporter construct (Promega). Transfection is performed using the Calcium Phosphate Transfection System according to the manufacturer's protocol (Life Technologies). However, all volumes are adjusted (divided by 6.05), since the protocol is described for a 100 mm tissue culture dish instead of a 6-well dish. The total amount of DNA is 3.3 microgram per well, and identical amounts of reporter DNA and carrier DNA are used. The precipitate is left for 24 hours on the wells.

After 24 hours, cells harvested with Trypsine/EDTA (Life Technologies) and collected in U2OS medium according to standard procedures. 5×10³ transfected U2OS cells are seeded per well in a 96-well plate in 100 μl of U2OS-medium and incubated overnight at 37° C. in a humidified incubator at 10% CO₂.

The next day, re-propagated viruses encoding potential hits (see above) and control viruses transducing E2F2, p16^{INK4a}, p27KIP, EGFP, LacZ, and Empty, are used to infect U2OS cells transiently transfected with the E2F-luciferase reporter construct, or the pGL3-basic or pGL3-promoter control reporter constructs. Cells are infected with the viruses at MOI of 100 and 500. 3 wells of a 96-well plate are used for each MOI for all viruses. Cells are incubated further for 48 hours at 37° C. in a humidified incubator at 10% CO₂.

48 hours after infection, the medium is pulled of from the wells. The cells are washed once with PBS and frozen away at −20° C. in 100 μl of PBS.

After thawing and resuspending of the cell lysate, 50 μl of each well is transferred to a Wallac Black&White sample plate and 50 μl of Steady-Glo (Promega) is added and incubated for 15 minutes at room temperature. Luciferase activity is determined on a Wallac Trilux 1450 microbeta Liquid Scintillation and Luminescence Counter.

Results are calculated as fold activation compared to empty virus.

All controls used in this assay gave good results in that E2F2 and E2F3 stimulate the E2F-luciferase reporter 4.5-9 times compared to empty virus while p16^{INK4a} and p27^{KIP} repressed luciferase activity 0.4-0.2 times compared to empty virus in a MOI-dependent manner. Other control viruses like EGFP hardly influenced luciferase activity.

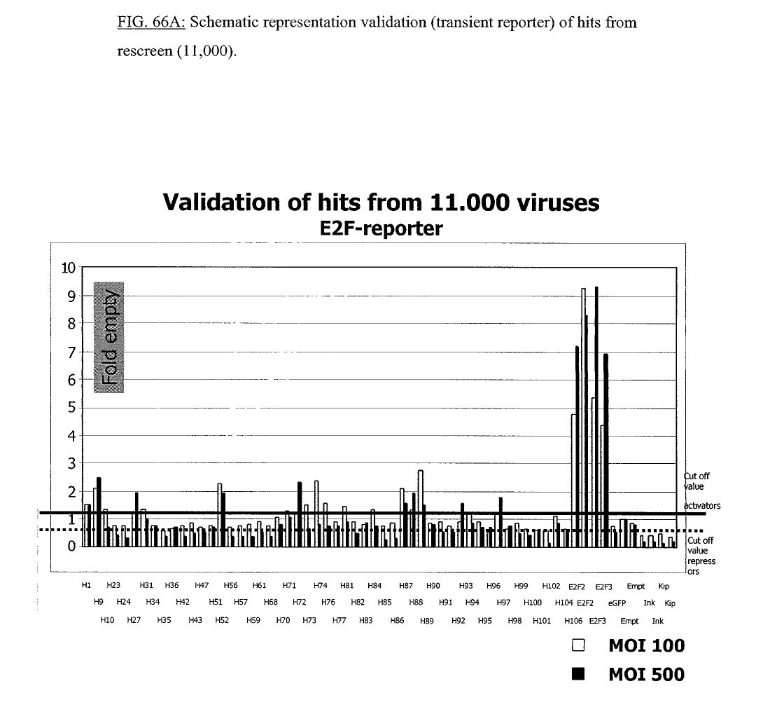

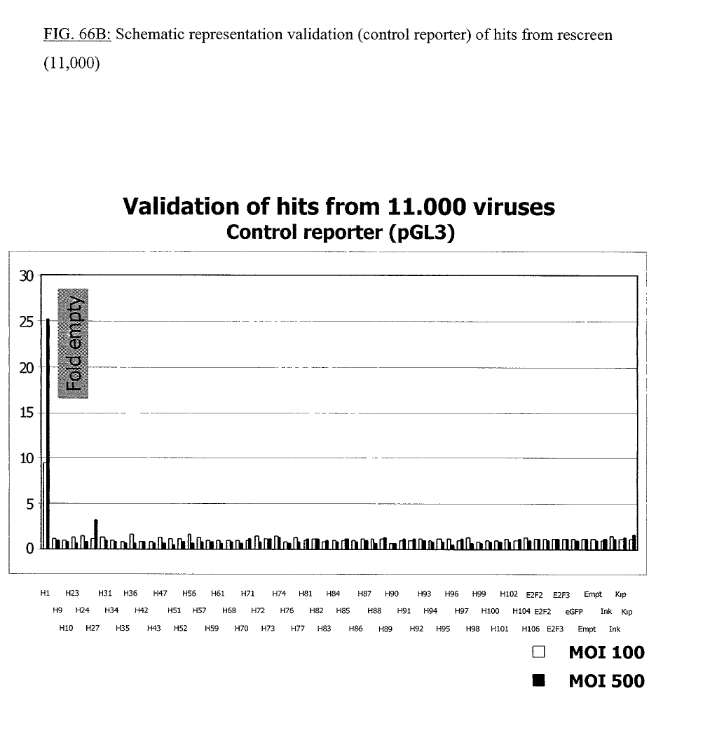

Of the potential hits tested (see FIG. 66A), one is retained that stimulates E2F-reporter activity more than 1.2 times the value of empty vector (H1-9), and which did not stimulate the pGL3-basic or pGL3-promoter control reporters (FIG. 66B and data not shown).

Two potential hits (H1 and H27) stimulate both the E2F reporter and the pGL3-basic control reporter (compare FIG. 66A and FIG. 66B), and are discarded. Two potential hits (H89 and H1-92) stimulate both the E2F reporter and the pGL3-promoter control reporter to equal relative levels and are also discarded.

Two potential hits are retained that repressed E2F-reporter activity more than 0.6 times empty vector control (H1-35 and H1-96), while not influencing the pGL3-basic or pGL3-promoter control reporters (see FIG. 66A). Several other potential repressors are discarded since they also seemed to influence the pGL3-promoter control reporter (data not shown).

### Example 10

### Sequence Identification of Validated Hits

For sequencing and sample tracking purposes, fragments of the cDNAs expressed by the hit adenoviruses are amplified by PCR using primers complementary to sequences flanking the MCS of the pAdapt plasmid. The following protocol is applied to obtain these PCR fragments. Day 0, Per.C6/E2A cells are seeded in 96-well plates at a density of 2.25×10⁴ cells per well, in 200 μl of Per.C6/E2A medium. Cells are incubated overnight at 39° C., 10% CO₂. Day 1, cells are infected with the hit viruses using 2 μl of crude cell lysate material from the repropagation step. Cells are then incubated at 34° C., 10% CO₂ until appearance of starting of CPE (as revealed by the swelling and rounding up of the cells, typically 2 to 3 days post infection). The supernatant is removed from the cells and 50 μl of lysis buffer (1× Expand High Fidelity buffer with MgCl₂ (Roche Molecular Biochemicals Cat No 1332465) supplemented with 1 mg/ml proteinase K (Roche Molecular Biochemicals Cat No 745 723) and 0.45% Tween-20 (Roche Molecular Biochemicals, Cat No 1335465) is added to the cells. Cell lysates are then transferred to sterile micro centrifuge tubes and incubated at 55° C. for 2 hrs followed by a 15 min inactivation step at 95° C. 5 μl of the cell lysates is then added to a PCR master mix composed of 5 μl 10× Expand High Fidelity buffer +MgCl₂, 1 μl of dNTP mix (10 mM for each dNTP), 1 μl of pClip-FOR primer (10 μM stock, sequence: 5′ GGT GGG AGG TCT ATA TAA GC), 1 μl of pAdapt-REV primer (10 μM stock, sequence: 5′ GGA CAA ACC ACA ACT AGA ATG C), 0.75 μl of Expand High Fidelity DNA polymerase (3.5 U/μl, Roche Molecular Biochemicals) and 36,25 μl of H₂O. PCR is performed using a PE Biosystems Gen Amp PCR system 9700 as follows: the PCR mixture (50μl in total) is incubated at 95° C. for 5 min; at 95° C. for 30 sec; 55° C. for 30 sec; 68° C. for 4 min, and this is repeated for 35 cycles. A final incubation at 68° C. is applied for 7 min. The amplification products are resolved on a 0.8% agarose gel containing 0,5 μg/ml ethidium bromide and their length estimated by comparison with the migration of a standard DNA ladder. For this purpose, 15 μl of PCR mixture is mixed with 10 μl of 6× gel loading Buffer. The PCR products obtained are also used as template for sequencing using the aforementioned pClip-FOR primer.

### Example 11

### Polynucleotides and Polypeptides of the Invention

The sequence analysis of the identified nucleic acid hits revealed both unknown and known polynucleotide sequences (Table 2). The nuclear receptor PPARgamma (H1-96), proto-oncogene FosB (H1-35), and the Cdk inhibitor p57^{KIP2} (#7) are isolated in the screenings of the present invention. They have already been described as regulators of E2F and therefore provide an internal control for the screening method of the present invention (Altiok, et al. (1997) Genes Dev. 11: 1987-98; Wakino, et al. (2000) J. Biol. Chem. 275:22435-41; Brown, et al. (1998) Mol. Cell. Biol. 18:5609-19; U.S. Pat. No. 6,008,323; Nakanishi, et al. (1999). Biochem.Biophys.Res.Commun. 263:35-40.).

TABLE 2
nucleic acid hits

| Hit | Modulator | SEQ. Similarity | SEQ ID NO |
|---|---|---|---|
| H1-35 | Repressor | FOS-B | |
| | | (NM_006732) | |
| H1-96 | Repressor | PPARgamma | |
| | | (U10374) | |
| #7 | Repressor | P57^{KIp2} | |
| | | (D64137) | |
| H1-9 | Activator | Hypothetical protein | 13, 14 |
| | | (NM_017710) | |

Features of hit H1-9

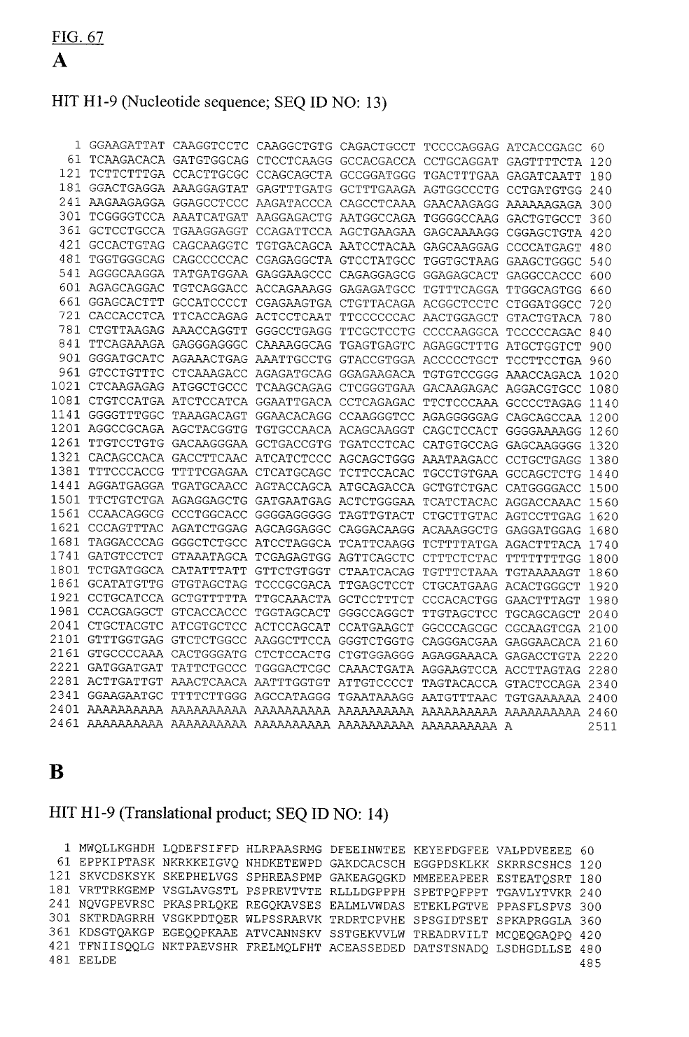

Hit H1-9 is detected as an activator in the E2F screen. According to a BLASTN search, the DNA sequence of hit H1-9 (FIG. 67A; SEQ ID NO: 13) is identical (100% identity) with the public cDNA sequence referenced by GenBank accession number NM_{—}017710. However, as compared to this public sequence, a 5′-terminal fragment of 571 bps is missing in H1-9. The predicted sequence referenced by GenBank accession number XM_{—}002079 gives a better match at the 5′ end of H1-9, although there are still 86 bps less in the H1-9 sequence.

Based on length and order of predicted ORFs, ORF number 1, encoding a protein of 485 amino acids, most likely will be the coding sequence of this cDNA (FIG. 67B; SEQ ID NO: 14). A search for homology in GenBank results in a perfect match with the amino acid sequence referenced by GenBank accession number NP_{—}060180, which contains the translated product of the ORF in NM_{—}017710. NP_{—}060180 describes a hypothetical protein, FLJ20203, of 697 amino acids. The N-terminal 212 amino acids of NP_{—}060180 are not encoded by ORF1. The C-terminal part gives a perfect match. On the other hand, the match with the sequence in XP_{—}002079 (containing the translated product of the ORF in XM_{—}002079) is 100%, both in sequence as in length.

Based on the annotations in GenBank, there is no functional information available for this sequence; motif database searches, e.g. on BLOCKS+, PFAM, and PROSITE, did neither give a clue to the function of the encoded protein.

Therefore, the finding, as disclosed in the present invention, that a new protein, encoded by the ORF of H1-9, positively regulates E2F activity, provides new and unexpected insights in the regulation of E2F-mediated activities, as well as new and unexpected insights in the function of H1-9 and possibly relating sequences such as the sequences referenced by NM 017710 and XM_{—}002079.

### Example 12

### Further Validation of Hit H1-9.

Due to the arrayed format of the adenoviral placenta library, positive hits can be tracked back to individual wells on the glycerol stock plates (see example 1). In this way, the glycerol stock of pIPspAdapt6 plasmids containing hit H1-9 is picked and grown in LB-amp. Following verification of the insert by restriction enzyme analysis, in comparison to the PCR product of the adenovirus H1-9 hit (see Example 10), and sequence analysis of the insert, a large-scale preparation of H1-9 in pIPspAdapt6 is purified on a Qiagen maxiprep column.

U2OS cells are grown in Dulbecco's modified eagle's medium containing 10% fetal calf serum (FBS) and supplemented with penicillin/streptomycin (100 units/ml; (Gibco-BRL) and glutamine (Gibco-BRL)(abbreviated U2OS medium) on T80 culture flasks until 50% confluence is reached. Cells are washed in PBS and trypsinized in 1 ml of Trypsin/EDTA (Gibco-BRL) for 5 minutes at 37° C. and collected in 10 ml of culture medium. Subsequently cells are washed in 10 ml of PBS and resuspended in electroporation buffer (2 mM HEPES (pH 7.2), 15 mM K₂HPO₄/KH₂PO₄, 250 mM mannitol, 1 mM MgCl₂,) at a concentration of 10⁷ cells per milliliter.

DNA mixes are prepared in a total volume of 10 μl containing reporter plasmid (5 μgram), effector plasmid (0 μgram, 0.5 μgram (only for H1-9 in pIPsAdapt6, or 2.5 μgram; adjusted to 2.5 μgram with empty pIPspAdapt6), and 0.1 μgram of Renilla (pRL-CMV; Promega). Reporter plasmids are either E2F-luciferase reporter construct, or the pGL3-promoter control reporter construct (Promega)(see Example 9). Effector plasmids are pIPspAdApt3-E2F2 (see Example 3); pIPspAdApt6-EGFP (see Example 3); or H1-9 in pIPspAdapt6 (see above).

In total, 9 DNA mixtures are prepared and added to 100 μl of the cell suspension. Electroporation is performed on a Gene Pulser II electroporator including RF module (BioRad) at 140 Volt, 40 Khertz, 1.5 millisecond per pulse, 1.5 second delay, total 15 pulses. Following electroporation, 900 μl of U2OS medium is added and 100 μl of the resulting mixture is plated per well of a 24-well plate in a final volume of 1 ml. Cell are incubated at 37° C. in a humidified incubator at 10% CO₂ for 40 hours.

After this period, cells are washed in 0.5 ml of PBS, and 100 μl of 1× Passive Lysis Buffer is added. Samples are further treated according to the Dual-Luciferase Reporter Assay System Kit (Promega). Luciferase and Renilla activity is determined on a Lumat LB9507 (EG&G, Berthold) luminometer.

As can be seen in FIG. 68, transfection of E2F2 induces the relative luciferase activity of the E2F-reporter, while the relative luciferase activity of the control reporter is not changed. Transfection of EGFP does not significantly modulate the relative expression of the E2F-reporter or the control reporter. Transfection of hit H1-9 also induces the relative luciferase activity of the E2F-reporter, while the relative luciferase activity of the control reporter is not changed, similar to E2F2. Higher amounts of plasmid, however, do not show a further increase in the relative luciferase levels, probably due to toxicity of the cells. We conclude that the hit H1-9 in pIPspAdapt6 is active and that transfection of this plasmid results in the specific activation of E2F.

### Example 13

### Analysis of Hits for Activity as Secreted Proteins

To analyze for secreted proteins that influence E2F activity, producer cells are infected by the viruses of the adenoviral library prepared as described hereinabove. Alternatively, the producer cells may be infected with viruses identified as “hits” in the E2F activity assay. Another population of cells are infected with control viruses that induce or do not induce E2F activity. The conditioned media from each infected producer cell population are harvested 2 or 4 days post infection (dpi) and added to freshly seeded primary human cells. If the conditioned medium contains secreted proteins that induce E2F activity, this will be identified after adding the conditioned medium to the cells and by analyzing E2F activity.

HeLa or U2OS producer cells are cultured in DMEM 10% FBS. 1000-5000 HeLa cells/well or 1000-5000 U2OS cells/well (384-well plate) are plated in 60 μl medium. Four hours later, the cells are infected with 1μl of adenoviral stock solutions. Two or 3 days later, 384-well plates containing 1000 primary cells/well are seeded in 30 μl of medium. One day after seeding the primary cells, the primary cells are infected with adenovirus containing the E2F-reporter of Example 16, below, using the conditions described in Example 17, below. One day later, 40 μl of the conditioned medium, harvested from the HeLa or U2OS producer cells is transferred to the corresponding well of the 384-well plates containing the cells, using the 96-channel Hydra dispenser. One day after transferring the supernatants, E2F activity is analysed in the primary cells.

### Example 14

### Human FAb Phase Display Selection of antibodies Against Validated Hits

Phage displaying human FAb fragments encompassing the light and heavy variable and constant regions are employed to isolate antibodies that bind to the protein identified herein (characterized by SEQ ID NO: 14). A human FAb phage display library is constructed in a phage display vector such as pCES1 a vector derived from pCANTAB6 (McCafferty, et al. (1994) Appl. Biochem. Biotech. 47:157-73). The library is constructed in the filamentous E. coli phage m13 and the FAb sequences are displayed as N-terminal fusion proteins with the minor coat protein pill. The library can have a complexity of more or less than 10¹⁰ different sequences.

Three types of targets can be used to select for polypeptide-displaying phages that bind to the amino acid epitopes encoded by the sequences of SEQ ID NO: 13.

First, a predicted extracellular or otherwise accessible domain encoded by sequences of SEQ ID NO: 13 is synthesized as a synthetic peptide. The N-terminus of this peptide is biotinylated and followed by three amino acid linker residues KRR, followed by the predicted sequence of encoded by sequences of SEQ ID NO: 13, respectively.

Second, a fusion protein is made of a portion of or the complete polypeptide encoded by sequences of SEQ ID NO: 13 in frame with the ORF of glutathione-S-transferase (GST) or maltose-binding protein or His6 or another tag and expressed in E. coli. Alternatively, a His6 or another tag is fused in frame with the ORF of SEQ ID NO: 13 and expressed in a mammalian expression system such as PER.C6/E2A. Fusion proteins are then purified using, for example, NiNTA columns for His6-tagged proteins (Qiagen) or glutathione resin (Pharmacia) for GST-tagged proteins.

To select for FAb displaying phages that bind to polypeptides encoded by sequences of SEQ ID NO: 13, the following selection procedure is employed. A pool of FAb displaying phage is selected out of a complex mixture of a high number of different FAb displaying phages in four rounds by their ability to bind with significant affinity to a biotinylated peptide or to a purified fusion protein that has been expressed in E. coli or in a mammalian expression system such as PER.C6/E2A. The collection of selected FAb displaying phage is further decreases by the next selection procedure: the FAb displaying phage are further selected in three rounds for their ability to bind to polypeptides encoded by sequences of SEQ ID NO: 13 present in cell lysates from cells overexpressing SEQ ID NO: 13. For selection on biotinylated peptide 250 μl of FAb library (or eluted phage from the previous round) is mixed with 250 μl 4% non fat dry milk in PBS and equilibrated while rotating at RT for 1 hour. Subsequently biotinylated peptide (20-500 nM in H₂O) is added. This mix is incubated on the rotator at RT for 1 hour before 250 ml equilibrated streptavidin-dynabeads in 2% non fat dry milk in PBS is added. After incubation on a rotator at RT for 15 min the beads with the bound phage are washed 5 times with PB S/2% non fat dry milkl/0.1% Tween, 5 times with PBS/0.1% Tween and 5 times with PBS. Then the phage are eluted by incubation with 0.1 M Triethylamine on a rotator at RT for 10 min and neutralised in 1 M Tris-HCl (pH 7.4).

The eluted phages are titered and amplified in E. coli bacteria, e.g. TG1, before the next selection.

The pools of the last various selection rounds are tested for binding to the biotinylated peptides or preferably the fusion or purified full length proteins in a specific ELISA and also for cell binding by flow cytometric analysis where appropriate. Once FAb displaying clones are isolated, double strand phagemid DNA is prepared and used to determine the nucleotide and deduced amino acid sequence of the displayed variable heavy and light chains.

The FAb phages or antibodies derived thereof are used as diagnostic tools, for example in immunohistochemistry, as research tools, for example in affinity chromatography, as therapeutic antibodies directly, or for the generation of therapeutic antibodies by generating anti-idiotypic antibodies.

### Example 15

### Screening for Compounds that Alter E2F Activity

Polynucleotides of SEQ ID NO: 13 or polypeptides of SEQ ID NO: 14 are attached to the bottom of the wells of a 96-well plate by incubating the polypeptide or polynucleotide in the wells overnight at 4° C. Alternatively, the wells are first coated with composition of polylysine that facilitates binding of the polypeptide or polynucleotides.

Following attachment of the biopolymer, samples from a library of test compounds are added to the wells and incubated for a sufficient time and temperature to facilitate binding using an appropriate binding buffer known in the art. Following this incubation, the wells are washed with an appropriate washing solution at 4° C. The stringency of the washing steps is varied by increasing or decreasing salt and/or detergent concentrations in the wash. Detection of binding is accomplished by using antibodies (RIA, ELISA), biotinylation, biotin-streptavidin binding, and radioisotopes. The concentration of the sample library compounds is also varied to calculate a binding affinity by Scatchard analysis.

Binding to the polypeptide or polynucleotides identifies a “lead compound”. Once a lead compound is identified the screening process is repeated using compounds chemically related to the lead compound to identify compounds with the tightest binding affinities. Selected compounds having binding affinity are further tested in one of the two following assays.

E2F Transcriptional Assay:

Compounds that bind to the polynucleotide or polypeptide are tested for their effects on E2F activity. In general, a cell that expresses a polynucleotide of SEQ ID NO: 13 is treated with a binding compound. The treatment with the compound can occur pre-transfection with the polynucleotide sequence (see day 0 and 1 below), post-transfection (see days 1 to 4 below), or concurrently with transfection (see day 1 below). After transfection and incubation with the compound, E2F activity is assessed.

On day 0, 1000 U2OS cells are seeded in 60 μl medium, in each well of a black 384-well plate with clear bottom (Costar or Nunc). One day later, control viruses or viruses comprising SEQ ID NO: 13 are added to the hCAR transfected wells according to the following procedure: Plates harbouring control viruses or SEQ ID NO: 13 are allowed to thaw at room temperature. Two μl of control virus or SEQ ID NO: 13 virus are transferred to the 384-well plate containing the U2OS cells using a Hydra100 96 channel dispenser. The viruses from the control plates are screened in duplicate, while the viruses from the PhenoSelect library are screened in singular fashion. The plates containing the freshly infected cells are then incubated at 37° C. Three days after infecting the cells, plates are analyzed for E2F activity. The binding compounds identified in the previous step can be added on Day 0, Day 1, or on any of the days after transfection with the virus containing SEQ ID NO: 13.

mRNA Expression Assay:

On day 0, 1000 U2OS cells are seeded in 60 μl medium in the wells of a black 3 84-well plate with clear bottom (Costar or Nunc). The cells are plated in duplicate so that RNA is isolated from a first set of plates while E2F activity is assessed in the second set of plates. One day later, the binding compound is added to the medium of both sets of plates at a concentration ranging from 1 nM to 1 mM. Three days after addition of the compound, the second set of plates is analyzed for E2F activity. One, two, or three days after addition of the compound, the cells of the first set are lysed and the RNA from the cells is extracted. Extraction is performed as described in Maniatis, et al. (1982) Molecular Cloning: A Laboratory Manual, 2nd ed., or alternatively a commercially available kit (e.g., Qiagen) is used. RNA isolated from the cells is used as template for PCR using primers specific to SEQ ID NO: 13 to determine if the compound induces mRNA expression.

As an alternative, the above experiment can be done at a larger scale in 96- or 24-well plates so that mRNA encoded by SEQ ID NO: 13 is isolated, and detected by RNase protection assay or northern blotting. Alternatively, cell lysates are isolated and subjected to SDS-PAGE electrophoresis, transferred to membranes, and immunoblotted to detect expression of polypeptides encoded by SEQ ID NO: 13.

### Example 16

### Generation of Adenoviral E2F-Reporter

Cell lines almost always have mutations in cell cycle regulatory pathways, which prevent the isolation of novel regulators that function upstream of the mutated proteins. Therefore, the use of an E2F-reporter in primary cells leads to the isolation of more hits from these screens, as primary cells in general do not have mutations in cell cycle regulatory pathways.

As the use of primary cells excludes the use of stable reporter cells, an adenoviral E2F-reporter construct is generated to use primary cells for the isolation novel modulators of E2F activity. The adenoviral E2F-reporter is co-infected into the targets cells together with the individual library viruses. An adenoviral E2F-reporter also allows using multiple primary cells during screening, which enhances the isolation of cell-specific modulators.

To generate an adenoviral E2F-reporter, the pGL3-TATA-E2F-luc construct is digested with SalI, which cuts 5′ to the E2F-dependent promoter, and NotI, which cuts downstream of the luciferase/poly(A) signal. The 5′ overhangs are blunted by filling in with Klenow polymerase enzyme in the presence of dNTPs. A pGL3-TATA-luc construct, without E2F-binding sites, is treated in a similar manner. To improve the separation of the insert from the vector, the vector fragment is further digested with XmnI. The insert fragments are isolated on a 0.8% agarose gel, and purified using a QIAquick gel extraction kit (Qiagen).

The adapter plasmid pIPspAdapt6 is digested with BglII, blunted by filling in with Klenow polymerase enzyme in the presence of dNTPs, and redigested with SnaBI. Following phosphatase treatment to prevent religation of the vector, the vector fragment is isolated on a 0.8% agarose gel, and purified using a QIAquick gel extraction kit (Qiagen).

Both insert fragments, from pGL3-TATA-E2F-luc and from pGL3-TATA-luc, are ligated to the adapter fragment using lx ligation buffer and T4 DNA ligase (New England Biolabs). Following transformation into E. coli and selection on ampicillin-agar plates, single colonies are inoculated to prepare miniprep DNA. Correct clones are obtained that contained the reporter fragments in both orientations in pIPspAdapt, as determined by restriction enzyme analysis and sequence analysis.

ΔE1/ΔE2A adenoviruses are generated from these adapter plasmids by co-transfection of the helper plasmid pWEAd5AflII-rITR.dE2A in PER.C6/E2A packaging cells, as described (WO99/64582).

Experiments, using the reporter viruses and control viruses transducing E2F3 and p16^{INK}, provide evidence that the counter clock-wise orientation, with transcription of the reporter in the direction of the left ITR, is most optimal. Therefore, this orientation is used in all further experiments.

### Example 17

### Optimization of Transient E2F Assay

A transient co-infection assay is developed for isolation of novel modulators of E2F activity in primary cells. Optimization of this new assay is done on U2OS wild type cells. These cells are co-infected with an adenoviral E2F-reporter (referred to as pGL3E2F reporter) and the ΔE1/ΔE2A control adenoviruses, as mentioned in example 3, transducing E2F3, p16^{INK4a} and EGFP. To study the specificity towards an E2F-dependent promoter, cells are also co-infected with the pGL3-TATA-luc reporter, which does not contain E2F binding sites (referred to as pGL3basic reporter). The cells are co-infected with reporter and control virus in different ratios to study optimal co-infection conditions.

U2OS wild type cells are seeded at 5×10³ cells per well in 96-well plates and incubated overnight at 37° C. in a humidified incubator at 10% CO₂ in 100 μl of DMEM supplemented with 10% heat inactivated FBS.

The next day, cells are co-infected with pGL3E2F reporter virus at MOIs of 250, 500 and 750 and control viruses at MOIs of 0, 250, 500 and 750. Each MOI of reporter virus is combined with each the four different MOIs of the control viruses. Empty virus is added to all samples in order to obtain a final total MOI of 1500. The final volume is set to 20 μl with culture medium. All experiments are performed in triplicate.

48 hours after infection, the medium is removed from the cells. 100 μl of Phosphate Buffered Saline (PBS; Gibco) is added to each well and the plates are frozen at −20° C.

After thawing and resuspension of the cell lysates, 50 μl is transferred to a Wallac Black&White sample plate. 50 μl of Steady-Glo luciferase (Promega) is added and within 7 hours luciferase activity is determined on a Wallac Trilux 1450 Microbeta Liquid Scintillation and Luminescence Counter.

To normalize for differences in protein content between wells, the CBQCA protein determination kit from Molecular Probes is used. All components of the CBQCA protein kit are prepared as described in the protocol.

A BSA standard curve is prepared as follows (differs from protocol):

| | | | |
|---|---|---|---|
| 12.5 μl of 4 mg/ml BSA + 37.5 μl PBS | → | 1 | μg/μl. |
| 10 μl of 4 mg/ml BSA + 40 μl PBS | → | 0.8 | μg/μl. |
| 25 μl of 0.8 mg/ml BSA + 25 μl PBS | → | 0.4 | μg/μl. |
| 25 μl of 0.4 mg/ml BSA + 25 μl PBS | → | 0.2 | μg/μl. |
| 25 μl of 0.2 mg/ml BSA + 25 μl PBS | → | 0.1 | μg/μl. |
| 0 μl of BSA + 25 μl PBS | → | 0 | μg/μl. |

Ten μl of each dilution is transferred to a fresh 96-well plate. Also 10 μl of the resuspended cell lysates (see above) is transferred to fresh 96-well plates. To each well 125 μl 0.1 M Sodium Borate, 5 μl 20 mM KCN and 10 μl 2 mM ATTO-TAG is added. The reactions are protected from the light by covering with aluminum foil. The plates are incubated for at least 1 hour (max. 5 hours) at RT with shaking. Fluorescence is measured on the FLUOstar Galaxy of BMG with excitation at 485/12 nm and emission at 525/20 nm. The optimal gain is set using the plate that contained the standard curve. All other plates are measured using the same gain.

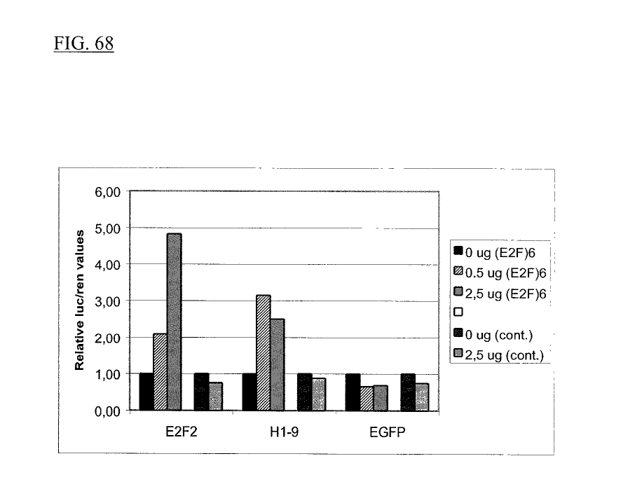

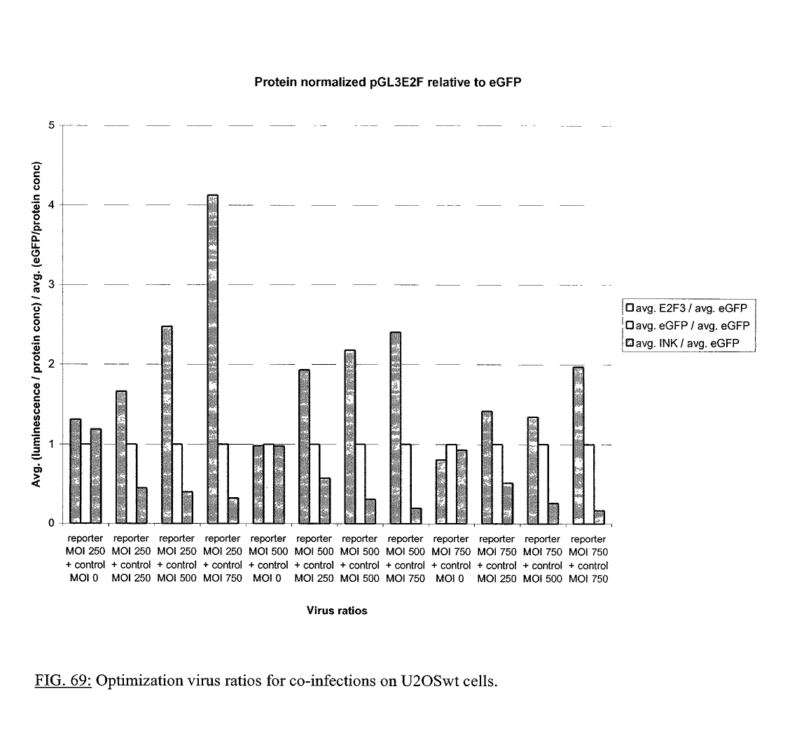

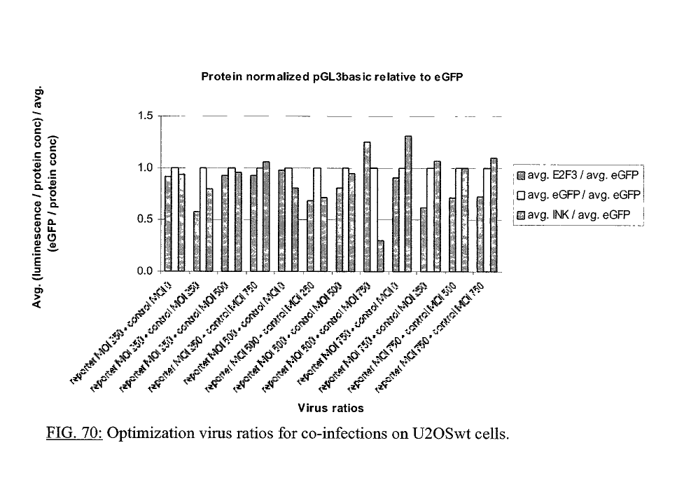

The results are expressed relative to the EGFP control after normalization for protein concentrations. As can be seen in FIG. 69 and FIG. 70, a clear induction by E2F3 and repression by p16^{INK4a} is observed at a MOI of 250 for the pGL3E2F reporter virus and a MOI of 750 for the control virus (FIG. 69). Under these conditions, no modulation of the pGL3-TATA-luc control reporter is observed (FIG. 70).

These ratios are used for all further experiments.

Because screens are normally performed using library viruses with unknown titers (using an assumed titer of 2×10⁹ vp/ml, there can be some variation between the wells in the total amount of virus that is added to the cells), to study the influence of different virus concentrations on the assay performance, a fill up experiment is performed. This assay is done on primary Human Uumbilical Vein Endothelial cells (HUVEC) using the conditions as described for U2OS wt cells.

HUVEC cells are seeded at 5×10³ cells per well in 96-well plates and incubated overnight at 37° C. in a humidified incubator at 10% CO₂ in 100 μl EBM-2 supplemented medium (Clonetics CC-4176).

The next day, cells are co-infected with pGL3E2F reporter virus at a known MOI of 250 and empty virus at a known MOI of 0, 500, 750, 1250, 2250, 4750 and 9750, all in a total volume of 20 μl. HUVEC cells are also co-infected with pGL3E2F reporter virus at a known MOI of 250 and control viruses at a known MOI of 750 for comparison. All conditions are performed in triplicate.

48 hours after infection, the medium is removed form the cells. 100 μl of Phosphate Buffered Saline (Gibco) is added to each well and the plates are frozen at −20° C.

After thawing and resuspension of the cell lysate, 50 μl is transferred to a Wallac Black&White sample plate. 50 μl of Steady-Glo luciferase (Promega) is added and within 7 hours luciferase activity is determined on a Wallac Trilux 1450 Microbeta Liquid Scintillation and Luminescence Counter.

All components of the CBQCA protein kit (Molecular Probes, C-6667) are prepared as described in the protocol.

A BSA standard curve is prepared as described before.

Ten μl of each dilution and 10 μl of the cell lysates are transferred to a fresh 96-well plate. To each well 125 μl 0.1 M Sodium Borate, 5 μl 20 mM KCN and 10 μl 2 mM ATTO-TAG is added. The reactions are protected from the light by covering with aluminum foil. The plates are incubated for at least 1 hour (max. 5 hours) at RT with shaking. Fluorescence is measured on the FLUOstar Galaxy of BMG with excitation at 485/12 nm and emission at 525/20 nm. The optimal gain is set using the plate that contained the standard curve. All other plates are measured using the same gain.

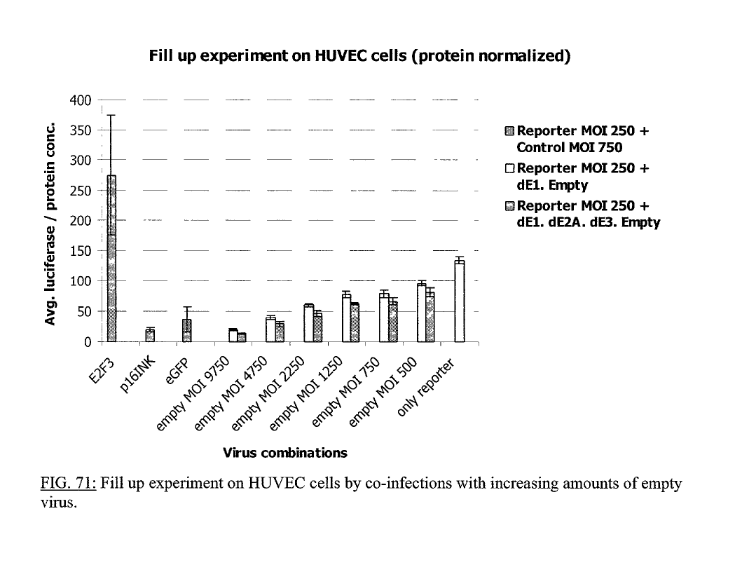

Results are expressed as average luminescence values normalized for protein concentrations (see FIG. 71).

A MOI dependent repression of the luciferase signal is observed when empty virus is added to the pGL3E2F reporter. Co-infection of empty virus with a known MOI of 4750 or higher leads to repression of the signal. Therefore library viruses with a real titer that is much higher than the assumed titer of 2×10⁹ vp/ml can be identified as false positive repressors. However, these false positive repressors are excluded after the rescreen that is done with real titers.

Wells that contain only reporter virus show a luminescence signal two times higher than wells that had been co-infected with empty virus at MOI 750. Real activation signals, like those observed after co-infection with E2F virus at MOI 750, show a three-fold higher luminescence signal than empty virus. The higher luminescence signal if no additional (library) virus is present may lead to the identification of false positive activators. These, however, are excluded by excluding results from wells that do not show Cyto Pathogenic Effects (CPE) after propagation of the viruses. These data are available from the adenoviral libraries. Moreover, these false positive activators are excluded after the rescreen that is done with real titers.

To determine the feasibility of the E2F-co-infection assay, 96 random viruses of the placenta library are picked and used to co-infect the HUVEC primary cells. These library viruses are previously used on the stable U2OS 1-5 E2F-reporter cell line and do not yield any positive or negative modulators.

To test these viruses, HUVEC cells are seeded at 5×10³ cells per well in 96-well plates and incubated overnight at 37° C. in a humidified incubator at 10% C0₂ in 100 μl EBM-2 supplemented medium (Clonetics CC-4176).

The next day, cells are co-infected with pGL3E2F reporter virus at a known MOI of 250 and library virus at a MOI of 750 based on an assumed titer of 2×10⁹ vp/ml for each virus. All infections are done in a total volume of 20 μl. HUVEC cells are also co-infected with pGL3E2F reporter virus at a known MOI of 250 and control viruses at a known MOI of 750 for comparison. All conditions are performed in duplicate.

48 hours after infection, the medium is removed from the cells. 100 μl of PBS (Gibco) is added to each well and the plates are frozen at −20° C.

After thawing and resuspension of the cell lysate, 50 μl is transferred to a Wallac Black&White sample plate. Fifty μl of Steady-Glo luciferase (Promega) is added and within 7 hours luciferase activity is determined on a Wallac Trilux 1450 Microbeta Liquid Scintillation and Luminescence Counter.

All components of the CBQCA protein kit (Molecular Probes, C-6667) are prepared as described in the protocol. A BSA standard curve is prepared as described before.

Ten μl of each BSA dilution and 10 μl of the cell lysates are transferred to a fresh 96-well plate. To each well 125 μl 0.1 M Sodium Borate, 5 μl 20 mM KCN and 10 μl 2 mM ATTO-TAG is added. The reactions are protected from the light by covering with aluminum foil. The plates are incubated for at least 1 hour (max. 5 hours) at RT with shaking. Fluorescence is measured on the FLUOstar Galaxy of BMG with excitation at 485/12 nm and emission at 525/20 nm. The optimal gain is set using the plate that contained the standard curve. All other plates are measured using the same gain.

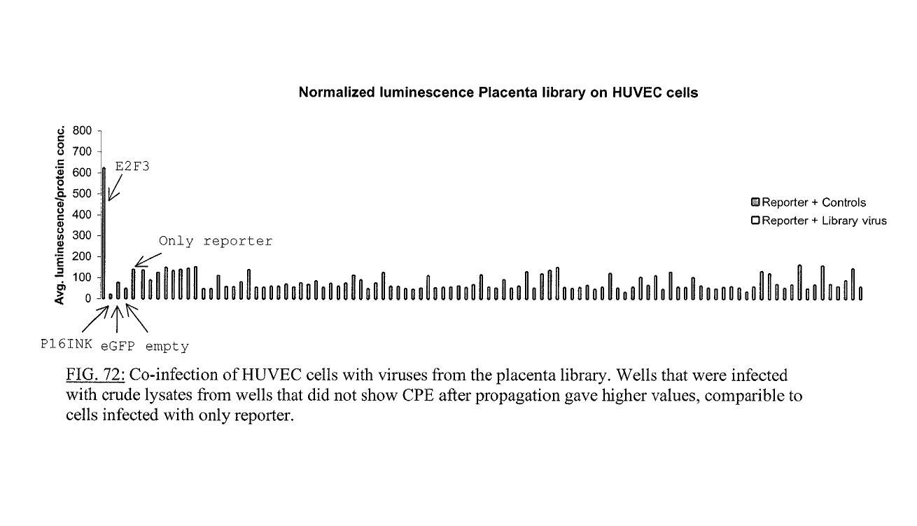

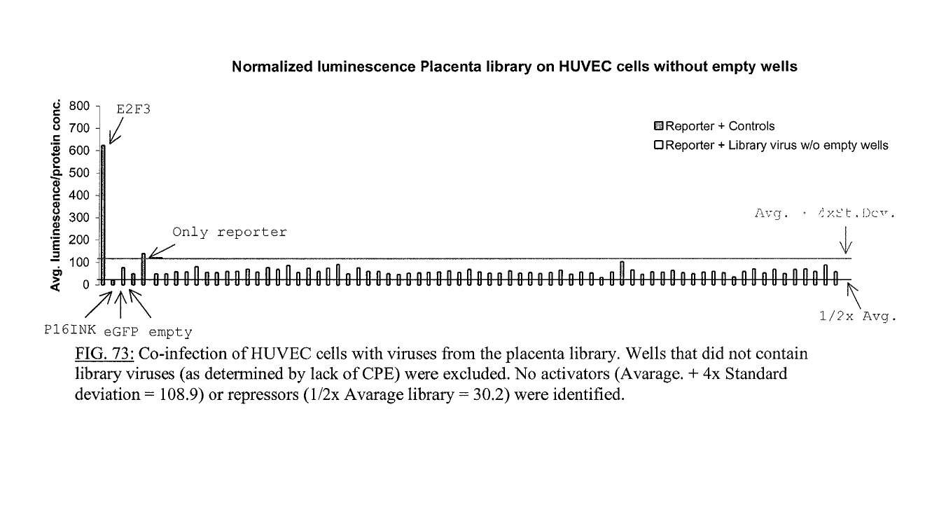

Results are expressed as average luminescence values normalized for protein concentrations (see FIG. 72 and FIG. 73). Empty virus gave an average luciferase reading of 48.9 relative light units (luminescence per microgram of protein). E2F3 expression causes a 12.8 times increase of the relative luciferase signal, compared to empty virus control. p16^{INK4a} expression causes a signal 2.3 times decreased as compared to empty virus control. Cells that are only infected with pGL3E2F reporter show to have a 2.9 times increase of the relative luciferase signal compared to empty virus control. This is also seen for wells that are co-infected with crude lysates from the placenta library from wells that do not show CPE. These wells are excluded from all calculations.

The average signal of the library is 60.3, with a standard deviation of 12.1.None of the library viruses induce readings higher than the average of the library plus 4 times the standard deviation, 108.9.

The lowest value measured for the library viruses is 32.8, which is still 1.5 times higher than the signal of p16^{INK4a}.

None of the library viruses induce values lower than V₂ times average of the library, 30.2.

Infection of Human Primary Cells Using Adenoviral Expression of hCAR

Primary human cells are sometimes difficult to transduce using Ad5 C01 because they lack or have a very low expression of the receptor that mediates the infection of the Ad5C01 viruses. To circumvent this problem, adenoviruses with different fiber protein variants are used that are able to infect efficiently primary cells. These viruses, Ad5C15 or Ad5C20, code for the human Coxsackievirus and Adenovirus Receptor (hCAR) (Bergelson, et al. (1997) Science 275(5304):1320-3). Transduction with these viruses and subsequent expression of the hCAR receptor makes cells competent to transduction with Ad5C01 virus. The use of Ad5C15-hCAR or Ad5C20-hCAR in double infections facilitates infection of primary cells using a much lower MOI for Ad5C01 than in a single infection.

The hCAR cDNA is isolated using a PCR methodology. The following hCAR-specific primers are used:

| | |
|---|---|
| HuCARfor | |
| 5′-GCGAAGCTTCCATGGCGCTCCTGCTGTGCTTC | (SEQ ID NO:15) |
| G-3′ | |
| HuCARrev | |
| 5′-GCGGGATCCATCTATACTATAGACCCATCCTTG | (SEQ ID NO:16) |
| CTC-3′. | |

The 5′ primer contains a HindIII site, and the 3′ primer a BamHI site. The hCAR cDNA is PCR amplified from a HeLa cell cDNA library (Quick clone, Clontech). A single fragment of 1119 bp is obtained and digested with the HindIII and BamHI restriction enzymes. pIPspAdapt6 vector (described in U.S. Pat. No. 6,340,595) is digested with the same enzymes, gel-purified and used to ligate to the digested PCR hCAR fragment.

The viruses described in this example have the Ad5 genome backbone with the E1A, E1B and E2A genes deleted. The viruses Ad5C15-hCAR and Ad5C20-hCAR have a fiber modification (C 15 or C20) and do not have the E2A gene deleted in their genome.

### Example 18

### In Vivo Analysis of Hits from the E2F Transcriptional Activation Screen

Down regulation and over expression of SEQ ID NO: 13 are tested in transgenic animal models.

For down regulating expression of SEQ ID NO: 13, knockout animals, preferably mice, are generated according to established procedures. One or more exons of the genes encoding SEQ ID NO: 13 are deleted by homologous recombination in mouse ES cells. These ES cells have been isolated from a limited number of homozygous strains of inbred lab mice well-suited to derive knock-out mice and are well known for those skilled in the art. Removal of one or more exons is checked by techniques such as southern blotting and the diploid state of ES cells is checked by cytogenetic techniques. Knockout ES cells harbouring the expected microdeletion and the expected number of chromosomes are then used to derive mice, according to established procedures. Resulting chimeric mice are then used to start a colony of knockout mice where the mice can be hetero- or homozygous for the allele in which one or more exons of the gene corresponding to SEQ IDNO: 13 are deleted. Both hetero- and homozygous knock-out mice are then used to study e.g. proliferation and apoptosis in the tissues of these mice, in comparison with wild-type mice, i.e. mice from the same inbred homozygous strain that have the gene corresponding to SEQ ID NO: 13 intact. The absence of expression of SEQ ID NO: 13 is studied by western blotting and northern blotting, performed on tissues, including spontaneous or induced tumor tissue of wild-type and knock-out animals.

For over expressing SEQ ID NO: 13 in vivo, preferably in mice, the following procedure is followed: subclone SEQ ID NO: 13 into a eukaryotic expression plasmid, downstream of a ubiquitously expressed promoter or, preferably, downstream of a promoter allowing for expression only in a specific compartment of the body. The plasmid containing the above-mentioned promoter and SEQ ID NO: 13 is then used to derive transgenic mice according to established procedures. Homozygous mouse strains, well suited to derive transgenic mice, such as the FVB strain are used. Exogenous expression of SEQ ID NO: 13 is analysed using southern blot, allowing an estimation of the copy number of the expression cassette, integrated in the mouse genome and also by northern or western blotting, e.g., with antibodies produced in example 14. The effect of the exogenous expression of SEQ ID NO: 13 on proliferation and apoptosis and on cancer is analyzed as described above for knockout animals.

### Example 19

### Transfection of Hematopoietic Stem Cells with Activators of E2F Activity

Hits that activate E2F (e.g., H1-9) and therefore stimulate proliferation of cells, are used to stimulate the proliferation of, for example, hematopoietic stem cells for gene therapy, e.g., for treating sickle cell anemia, thalassemia, or severely combined immunodeficiencies (SCID). Hematopoietic stem cells represent attractive targets for genetic modification since their progeny make up the entire spectrum of the hematopoietic system. However, due to the inherent quiescent nature of stem cells, gene transfer is limited since stable integration of retroviruses, the most currently used expression and transfer system in gene therapy, requires cell division. Moreover, there is a need for increasing the proportion of genetically modified stem cells through ex vivo expansion before transplanting them back into the bone marrow. Furthermore, methodology for enriching pluripotent stem cells in culture could also have a major impact on treatment of blood and immune system disorders. For example, bone marrow transplantation is often the only option for persons having hematopoietic and immune system dysfunctions causes by chemotherapy or radiation therapy. Therefore, expansion of primitive stem cells in culture is a major advance for all aspects of bone marrow transplantation as well as gene therapy applications.

For this, CD34+ positive cells are infected with adenoviruses (International Application No. PCT/EPO1/11086) transducing H1-9 sequence, which activates E2F. CD34 does not appear on normal, mature human lymphoid or myeloid cells and is used for the identification of early progenitor and stem cells of the human hematopoietic system. Expression of H1-9 sequence induces proliferation of the CD34+ cells. The thus expanded CD34+ population are subsequently used to reconstitute the bone marrow.

One major advantage is that the proliferation of the CD34+ cells in vitro guarantees a more efficient transfer and integration of retroviruses for gene therapy purposes.

As the adenovirus does not integrate into the genome, an adenoviral infection is always transient as the adenoviral DNA gets degraded in the target cells and is gradually lost from the target cells. Therefore, expression of the H1-9 sequence decreases and disappears in time, resulting in normal proliferating cells that respond to physiological signals after transplantation into the bone marrow.

An alternative to adenoviral infection is retroviral infection. For this, the H1-9 sequence that stimulates E2F activity is recloned in a retroviral vector. Retroviral particles, obtained after transfection in a retroviral packaging cell line, are used to infect the CD34+ cells. However, as the retrovirus integrates into the genome of the target cells, this leads to the stable expression of the transgene, which is not shut down. As this is not an optimal situation, the retrovirus is equipped with an inducible promoter such that expression is shut down after transplantation into the bone marrow.

It is advantageous for the H1-9 sequence to function only in some cells types and not in other cells types. This allows the in vivo use of viruses transducing this sequence, for example in tissue repair (e.g., bone repair and bone replacement) and corrective surgery, without the need to purify the target cells away from cells that are not allowed to proliferate.

All publications and patent applications are herein incorporated by reference to the same extent as if each individual publication or patent application is specifically and individually indicated to be incorporated by reference.

The invention now having been fully described, it will be apparent to one of ordinary skill in the art that many changes and modifications may be made thereto without departing from the spirit or scope of the appended claims.

We claim:

1. A method for identifying a unique nucleic acid capable of altering E2F activity in a cell, wherein said unique nucleic acid is present in a library, said method comprising:
(a) providing a library of a multitude of unique expressible nucleic acids, said library including a multiplicity of compartments, each of said compartments consisting essentially of one or more adenoviral vector comprising at least one unique nucleic acid of said library in an aqueous medium, wherein said adenoviral vector is capable of introducing said nucleic acid into a host cell, is capable of expressing the product of said nucleic acid in said host cell, and is deleted in a portion of the adenoviral genome necessary for replication thereof in said host cell;
(b) transducing a multiplicity of host cells with at least one adenoviral vector comprising at least one unique nucleic acid from said library;
(c) incubating said host cells to allow expression of the product of said nucleic acid; and
(d) determining if E2F activity is altered in said cell.

2. The method of claim 1 wherein step (d) comprises observing said host cell to assess E2F activity in said host cell relative to a host cell that has not been transduced with an adenoviral vector comprising said nucleic acid.

3. The method of claim 1 wherein said E2F activity is detected by measuring the activity of a gene product encoded by a polynucleotide operably linked to E2F binding sites.

4. The method of claim 3 wherein said polynucleotide operably linked to E2F binding sites encodes for a luciferase gene.

5. The method of claim 1 further comprising transducing the host cells in step (b) with an adenovirus encoding the receptor for adenovirus subtype 5(hCAR) wherein said receptor is expressed in said host cell.

6. A method according to claim 3 for determining whether the expression product of a nucleic acid, capable of inducing proliferation or apoptosis of a cell transfected with said nucleic acid, is secreted by said cell, further comprising:
(e) combining medium in which said cells are contained with a second population of cells that have not been infected with said vector and that contain an E2F reporter construct; and
(f) determining any alteration in E2F activity in said second population of cells.

7. A method according to claim 6 for determining whether the expression product of a nucleic acid is secreted by said cell and is capable of inducing proliferation or apoptosis of a cell, wherein said cells contain an E2F reporter construct, comprising:
(g) combining medium, from said cells in which E2F activity is detected, and in which said cells are contained, with said second population of cells.

8. The method of claim 3 wherein said second population of cells are primary human cells.

9. A method for identifying a unique nucleic acid capable of altering expression of E2F-inducible genes of a cell, wherein said unique nucleic acid is present in a library, said method comprising:
(a) growing a plurality of cell cultures containing at least one cell, said one cell expressing adenoviral sequence consisting essentially of E1-region sequences and expressing one or more functional gene products encoded by at least one adenoviral region selected from an E2A region and an E4 region; and
(b) transfecting, under conditions whereby said recombinant adenovirus vector library is produced, said at least one cell in each of said plurality of cell cultures with
i) an adapter plasmid comprising adenoviral sequence coding, in operable configuration, for a functional Inverted Terminal Repeat, a functional encapsidation signal, and sequences sufficient to allow for homologous recombination with a first recombinant nucleic acid, and not coding for E1 region sequences which overlap with E1 region sequences in said at least one cell, for E1 region sequences which overlap with E1 region sequences in a first recombinant nucleic acid, for E2B region sequences other than essential E2B sequences, for E2A region sequences, for E3 region sequences and for E4 region sequences, and further comprises a unique nucleic acid sequence and promoter operatively linked to said unique nucleic acid sequence; and
ii) a first recombinant nucleic acid comprising adenoviral sequence coding, in operable configuration, for a functional adenoviral Inverted Terminal Repeat and for sequences sufficient for replication in said at least one cell, but not comprising adenoviral E1 region sequences which overlap with E1 sequences in said at least one cell, and not comprising E2A region sequences or E4 region sequences expressed in said plurality of cells which would otherwise lead to production of replication competent adenovirus wherein said first recombinant nucleic acid has sufficient overlap with said adapter plasmid to provide for homologous recombination resulting in production of recombinant adenoviral vectors in said at least one cell;
(c) incubating said plurality of cells under conditions which result in the lysis of said plurality of cells facilitating the release of said recombinant adenoviral vectors containing said unique nucleic acid; and
(d) transferring an aliquot of said adenoviral vectors into a corresponding plurality of host cell cultures consisting of cells in which said vectors do not replicate, but in which said nucleic acids are expressible;
(e) incubating said host cells to allow expression of the product of said nucleic acid; and
(f) observing said host cell for a change in expression of genes operably linked to E2F binding sites.

10. A method according to claim 9, wherein said E2F activity is detected by measuring the activity of a gene product encoded by a polynucleotide operably linked to E2F binding sites.

11. A method according to claim 10, wherein said host cell is a neoplastic cell.

12. A method according to claim 11, wherein said nucleic acid induces an increase in E2F activity and apoptosis in said neoplastic cells.

13. A method according to claim 12, wherein said nucleic acid is transfected into a second population of primary host cells.

14. A method according to claim 13, wherein said primary host cells survive and do not proliferate abnormally.

15. A method according to claim 11, wherein said nucleic acid induces an increase in E2F activity and does not induce apoptosis in said neoplastic cells

16. A method according to claim 15, wherein said nucleic acid is transfected into a second population of primary host cells.

17. A method according to claim 16, wherein said primary host cells survive and do not proliferate abnormally.

18. A method for identifying a drug candidate compound useful in the treatment of apoptosis-associated disorders, said method comprising:
(a) contacting one or more test compounds with a polynucleotide comprising a sequence of SEQ ID NO: 13,
(b) determining the binding affinity of said one or more test compound to said polynucleotide,
(c) contacting a first subpopulation of host cells transfected with said polynucleotide with one or more of said test compound that exhibits binding affinity for said polynucleotide, and
(d) identifying, from said one or more test compounds, a candidate compound that inhibits expression of genes operably linked to E2F binding sites in said first subpopulation of host cells relative to a second subpopulation of said transfected host cells that have not been contacted with said candidate compound.

19. A method according to claim 18 wherein said test compound comprises a polynucleotide or a polypeptide.

20. A method for identifying a drug candidate compound useful in the treatment of apoptosis-associated disorders, said method comprising:
(a) contacting one or more test compound with a polypeptide expression product encoded by the polynucleotide comprising a sequence of SEQ ID NO: 13,
(b) determining the binding affinity of said test compound to said polypeptide,
(c) contacting a first subpopulation of host cells transfected with a polynucleotide expression vector coding for said polypeptide with one or more of said test compound that exhibits binding affinity for said polypeptide, and
(d) identifying, from said one or more test compounds, a candidate compound that inhibits expression of genes operably linked to E2F binding sites in said first subpopulation of host cells relative to a second subpopulation of said transfected host cells that have not been contacted with said candidate compound.

21. A method according to claim 20 wherein said test compound is contacted with a polypeptide comprising a sequence of SEQ ID NO: 14.

22. A method for identifying a drug candidate compound useful in the treatment of apoptosis-associated disorders comprising:
(a) contacting one or more test compounds with a corresponding number of one or more first subpopulations of host cell transfected with an expression vector encoding a polynucleotide comprising a sequence of SEQ ID NO: 13.

23. A method according to claim 22 further comprising
(b) selecting, from said one or more test compounds, a candidate compound that inhibits expression of genes operably linked to E2F binding sites in said first subpopulation of host cell relative to a second subpopulation of said transfected host cell that have not been contacted with any test compound.

24. A method according to claim 23 comprising
(b) selecting a candidate compound that results in a decrease in the expression of mRNA encoded by a polynucleotide comprising a sequence of SEQ ID NO: 13 in said first subpopulation of host cell relative to the expression of said mRNA in a second subpopulation of transfected host cells that has not been contacted with any test compound.

25. A method according to claim 24, wherein said host cell is a neoplastic cell.

26. A method according to claim 25, wherein said polynucleotide induces an increase in E2F activity and apoptosis in said neoplastic cells.

27. A method according to claim 26, wherein said polynucleotide is transfected into a second population of primary host cells.

28. A method according to claim 27, wherein said primary host cells survive and do not proliferate abnormally.

29. A method according to claim 25, wherein said nucleic acid induces an increase in E2F activity and does not induce apoptosis in said neoplastic cells

30. A method according to claim 29, wherein said nucleic acid is transfected into a second population of primary host cells.

31. A method according to claim 30, wherein said primary host cells survive and do not proliferate abnormally.

32. A method for identifying a drug candidate compound useful in the treatment of apoptosis-associated disorders, said method comprising:
(a) contacting one or more test compound with a polynucleotide comprising a sequence of SEQ ID NO: 13,
(b) determining the binding affinity of said one or more test compound to said polynucleotide,
(c) contacting a first subpopulation of host cells transfected with said polynucleotide with one or more of said test compound that exhibits binding affinity for said polynucleotide, and
(d) identifying, from said one or more test compounds, a candidate compound that increases expression of genes operably linked to E2F binding sites in said first subpopulation of host cells relative to a second subpopulation of said transfected host cells that have not been contacted with said candidate compound.

33. A method according to claim 32 wherein said test compound comprises a polynucleotide or a polypeptide.

34. A method for identifying a drug candidate compound useful in the treatment of apoptosis-associated disorders, said method comprising:
(a) contacting one or more test compound with a polypeptide expression product encoded by the polynucleotide comprising a sequence of SEQ ID NO: 13,
(b) determining the binding affinity of said test compound to said polypeptide,
(c) contacting a first subpopulation of host cells transfected with a polynucleotide expression vector coding for said polypeptide with one or more of said test compound that exhibits binding affinity for said polypeptide, and
(d) identifying, from said one or more test compounds, a candidate compound that increases expression of genes operably linked to E2F binding sites in said first subpopulation of host cells relative to a second subpopulation of said transfected host cells that have not been contacted with said candidate compound.

35. A method according to claim 34 wherein said polypeptide comprises a sequence of SEQ ID NO: 14

36. A method for identifying a drug candidate compound useful in the treatment of apoptosis-associated disorders comprising:
(a) contacting one or more test compounds with a corresponding number of one or more first subpopulations of host cell transfected with an expression vector encoding a polynucleotide comprising a sequence of SEQ ID NO: 13.

37. A method according to claim 36 further comprising
(b) selecting, from said one or more test compounds, a candidate compound that increases expression of genes operably linked to E2F binding sites in said first subpopulation of host cell relative to a second subpopulation of said transfected host cell that have not been contacted with any test compound.

38. A method according to claim 36 comprising
(b) selecting a candidate compound that results in an increase in the expression of mRNA encoded by a polynucleotide comprising a sequence of SEQ ID NO: 13 in said first subpopulation of host cell relative to the expression of said mRNA in a second subpopulation of transfected host cells that has not been contacted with any test compound.

39. A method according to claim 38 wherein said cells are primary cells.

40. A method for identifying a compound that inhibits proliferation of a neoplastic cell, said method comprising:
(a) contacting a compound identified by the method of claim 37 with a neoplastic cell; and
(b) observing an increase in apoptosis or a decrease in proliferation of said neoplastic cell.

41. A method for identifying a candidate compound that inhibits proliferation of a neoplastic cell, said method comprising:
(a) contacting a compound identified by the method of claim 37 with a neoplastic cell;
(b) contacting a compound identified by the method of claim 37 with a primary cell; and
(c) observing an increase in apoptosis or a decrease in proliferation of said neoplastic cell relative to said primary cell.

42. An expression vector comprising a sequence of SEQ ID NO: 13 wherein said vector is capable of expressing said polynucleotide.

43. An expression vector comprising a sequence complementary to a sequence of SEQ ID NO: 13 wherein said vector is capable of expressing said polynucleotide.

44. A pharmaceutical composition comprising a polypeptide expression product encoded by the polynucleotide comprising a sequence of SEQ ID NO: 13 and a pharmaceutically acceptable carrier.

45. A pharmaceutical composition comprising an expression vector according to claim 42 and a pharmaceutically acceptable carrier.

46. A pharmaceutical composition comprising an expression vector according to claim 43 and a pharmaceutically acceptable carrier.

47. The method of claim 1, wherein said adenoviral vector further comprises adenovirus genomic sequence encoding adenoviral fiber proteins from at least two serotypes of adenovirus.


SEQUENCE LISTING (16 sequences)

SEQ ID NO: 1 - DNA, 21 bp, Artificial Sequence
  Primer
cgtgtagtgtatttatacccg

SEQ ID NO: 2 - DNA, 21 bp, Artificial Sequence
  Primer
tcgtcactgggtggaaagcca

SEQ ID NO: 3 - DNA, 21 bp, Artificial Sequence
  Primer
tacccgccgtcctaaaatggc

SEQ ID NO: 4 - DNA, 21 bp, Artificial Sequence
  Primer
gcctccatggaggtcagatgt

SEQ ID NO: 5 - DNA, 20 bp, Artificial Sequence
  Primer
gcttgagcccgagacatgtc

SEQ ID NO: 6 - DNA, 24 bp, Artificial Sequence
  Primer
cccctcgagctcaatctgtatctt

SEQ ID NO: 7 - DNA, 27 bp, Artificial Sequence
  Primer
gggggatccgaacttgtttattgcagc

SEQ ID NO: 8 - DNA, 25 bp, Artificial Sequence
  Primer
gggagatctagacatgataagatac

SEQ ID NO: 9 - DNA, 27 bp, Artificial Sequence
  Primer
gggagatctgtactgaaatgtgtgggc

SEQ ID NO: 10 - DNA, 24 bp, Artificial Sequence
  Primer
ggaggctgcagtctccaacggcgt

SEQ ID NO: 11 - DNA, 45 bp, Artificial Sequence
  Primer
gtacactgacctagtgccgcccgggcaaagcccgggcggcactag

SEQ ID NO: 12 - DNA, 262 bp, Artificial Sequence
  E2F binding sites
ggtaccgagctcttacgcgtgctagcccttttaagcgcgaaactctacatttttcgcgaaactagtttcgcgcttaaaatcgtagagtttcgcgcttaaaaagtttcgcgcttaaaatcgtagagtttcgcgcttaaaaagtttcgcgcttaaaatcgtagagtttcgcgcttaaaattttaagcgcgaaactctacgattttaagcgcgaaactgggctcgagatctgggtatataatggatctgcgatctaagtaagctt

SEQ ID NO: 13 - DNA, 2511 bp, Human
ggaagattatcaaggtcctccaaggctgtgcagactgccttccccaggagatcaccgagctcaagacacagatgtggcagctcctcaagggccacgaccacctgcaggatgagttttctatcttctttgaccacttgcgcccagcagctagccggatgggtgactttgaagagatcaattggactgaggaaaaggagtatgagtttgatggctttgaagaagtggccctgcctgatgtggaagaagaggaggagcctcccaagatacccacagcctcaaagaacaagaggaaaaaagagatcggggtccaaaatcatgataaggagactgaatggccagatggggccaaggactgtgcctgctcctgccatgaaggaggtccagattccaagctgaagaagagcaaaaggcggagctgtagccactgtagcagcaaggtctgtgacagcaaatcctacaagagcaaggagccccatgagttggtgggcagcagcccccaccgagaggctagtcctatgcctggtgctaaggaagctgggcagggcaaggatatgatggaagaggaagccccagaggagcgggagagcactgaggccacccagagcaggactgtcaggaccaccagaaagggagagatgcctgtttcaggattggcagtggggagcactttgccatcccctcgagaagtgactgttacagaacggctcctcctggatggcccaccacctcattcaccagagactcctcaatttccccccacaactggagctgtactgtacactgttaagagaaaccaggttgggcctgaggttcgctcctgccccaaggcatcccccagacttcagaaagagagggagggccaaaaggcagtgagtgagtcagaggctttgatgctggtctgggatgcatcagaaactgagaaattgcctggtaccgtggaaccccctgcttccttcctgagtcctgtttcctcaaagaccagagatgcagggagaagacatgtgtccgggaaaccagacactcaagagagatggctgccctcaagcagagctcgggtgaagacaagagacaggacgtgccctgtccatgaatctccatcaggaattgacacctcagagacttctcccaaagcccctagagggggtttggctaaagacagtggaacacaggccaagggtccagagggggagcagcagccaaaggccgcagaagctacggtgtgtgccaacaacagcaaggtcagctccactggggaaaaggttgtcctgtggacaagggaagctgaccgtgtgatcctcaccatgtgccaggagcaaggggcacagccacagaccttcaacatcatctcccagcagctgggaaataagacccctgctgaggtttcccaccgttttcgagaactcatgcagctcttccacactgcctgtgaagccagctctgaggatgaggatgatgcaaccagtaccagcaatgcagaccagctgtctgaccatggggaccttctgtctgaagaggagctggatgaatgagactctgggaatcatctacacaggaccaaacccaacaggcgccctggcaccggggagggggtagttgtactctgcttgtacagtccttgagcccagtttacagatctggagagcaggaggccaggacaaggacaaaggctggaggatggagtaggacccaggggctctgccatcctaggcatcattcaaggtcttttatgaagactttacagatgtcctctgtaaatagcatcgagagtggagttcagctcctttctctacttttttttggtctgatggcacatatttattgttctgtggtctaatcacagtgtttctaaatgtaaaaagtgcatatgttggtgtagctagtcccgcgacattgagctcctctgcatgaagacactgggctcctgcatccagctgtttttattgcaaactagctcctttctcccacactgggaactttagtccacgaggctgtcaccaccctggtagcactgggccaggctttgtagctcctgcagcagctctgctacgtcatcgtgctccactccagcatccatgaagctggcccagcgccgcaagtcgagtttggtgaggtctctggccaaggcttccagggtctggtgcagggacgaagaggaacacagtgccccaaacactgggatgctctccactgctgtggagggagaggaaacagagacctgtagatggatgattattctgccctgggactcgccaaactgataaggaagtccaaccttagtagacttgattgtaaactcaacaaatttggtgtattgtccccttagtacaccagtactccagaggaagaatgcttttcttgggagccatagggtgaataaaggaatgtttaactgtgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - protein, 485 aa, Human [sequence omitted]

SEQ ID NO: 15 - DNA, 33 bp, Artificial Sequence
  Artificial primer with HindIII site
gcgaagcttccatggcgctcctgctgtgcttcg

SEQ ID NO: 16 - DNA, 36 bp, Artificial Sequence
  Artificial primer with BamHI site
gcgggatccatctatactatagacccatccttgctc